SEQUENCE LISTING (1106 sequences)

SEQ ID NO: 1 - protein, 11 aa, Artificial Sequence
  Made in a lab
SGEKLGDIYAS

SEQ ID NO: 2 - protein, 7 aa, Artificial Sequence
  Made in a lab
QDTKRPS

SEQ ID NO: 3 - protein, 8 aa, Artificial Sequence
  Made in a lab
QAWDSSTV

SEQ ID NO: 4 - protein, 14 aa, Artificial Sequence
  Made in a lab
SGTDSDVGGYNHVS

SEQ ID NO: 5 - protein, 7 aa, Artificial Sequence
  Made in a lab
DVDHRPS

SEQ ID NO: 6 - protein, 10 aa, Artificial Sequence
  Made in a lab
SSYRSGSTYV

SEQ ID NO: 7 - protein, 11 aa, Artificial Sequence
  Made in a lab
RASQSISSYLN

SEQ ID NO: 8 - protein, 7 aa, Artificial Sequence
  Made in a lab
AASALQS

SEQ ID NO: 9 - protein, 9 aa, Artificial Sequence
  Made in a lab
QQSYSPPVT

SEQ ID NO: 10 - protein, 11 aa, Artificial Sequence
  Made in a lab
RASQSISSWLA

SEQ ID NO: 11 - protein, 7 aa, Artificial Sequence
  Made in a lab
KASSLES

SEQ ID NO: 12 - protein, 8 aa, Artificial Sequence
  Made in a lab
QQYNSYWT

SEQ ID NO: 13 - protein, 11 aa, Artificial Sequence
  Made in a lab
RASQSVSSYLA

SEQ ID NO: 14 - protein, 7 aa, Artificial Sequence
  Made in a lab
DASNRAT

SEQ ID NO: 15 - protein, 10 aa, Artificial Sequence
  Made in a lab
QQRSNWPPFT

SEQ ID NO: 16 - protein, 11 aa, Artificial Sequence
  Made in a lab
QTSQSIDRYLN

SEQ ID NO: 17 - protein, 7 aa, Artificial Sequence
  Made in a lab
AASSLQS

SEQ ID NO: 18 - protein, 9 aa, Artificial Sequence
  Made in a lab
QQSYRTPRT

SEQ ID NO: 19 - protein, 12 aa, Artificial Sequence
  Made in a lab
RASQSVSSSYLA

SEQ ID NO: 20 - protein, 7 aa, Artificial Sequence
  Made in a lab
GASSRAT

SEQ ID NO: 21 - protein, 10 aa, Artificial Sequence
  Made in a lab
QQYGSSPLWT

SEQ ID NO: 22 - protein, 11 aa, Artificial Sequence
  Made in a lab
RASQSISSYLN

SEQ ID NO: 23 - protein, 7 aa, Artificial Sequence
  Made in a lab
AASSLQS

SEQ ID NO: 24 - protein, 9 aa, Artificial Sequence
  Made in a lab
QQSYSTPRT

SEQ ID NO: 25 - protein, 11 aa, Artificial Sequence
  Made in a lab
RASQSISTSLA

SEQ ID NO: 26 - protein, 8 aa, Artificial Sequence
  Made in a lab
YDASKRAS

SEQ ID NO: 27 - protein, 11 aa, Artificial Sequence
  Made in a lab
QLRINWPPEFT

SEQ ID NO: 28 - protein, 11 aa, Artificial Sequence
  Made in a lab
RASRGVITWLN

SEQ ID NO: 29 - protein, 7 aa, Artificial Sequence
  Made in a lab
GASTLQT

SEQ ID NO: 30 - protein, 9 aa, Artificial Sequence
  Made in a lab
QQTHSFPLT

SEQ ID NO: 31 - protein, 11 aa, Artificial Sequence
  Made in a lab
SGDKLGEKYVS

SEQ ID NO: 32 - protein, 7 aa, Artificial Sequence
  Made in a lab
QDTKRPS

SEQ ID NO: 33 - protein, 9 aa, Artificial Sequence
  Made in a lab
QTWDSDTYV

SEQ ID NO: 34 - protein, 11 aa, Artificial Sequence
  Made in a lab
RASQSISSYLN

SEQ ID NO: 35 - protein, 7 aa, Artificial Sequence
  Made in a lab
AASSLQS

SEQ ID NO: 36 - protein, 9 aa, Artificial Sequence
  Made in a lab
QQSYSTLWT

SEQ ID NO: 37 - protein, 11 aa, Artificial Sequence
  Made in a lab
RASQSVRSYLA

SEQ ID NO: 38 - protein, 7 aa, Artificial Sequence
  Made in a lab
DASNRAT

SEQ ID NO: 39 - protein, 9 aa, Artificial Sequence
  Made in a lab
QQYDNLPLT

SEQ ID NO: 40 - protein, 11 aa, Artificial Sequence
  Made in a lab
RTSQSVATYVN

SEQ ID NO: 41 - protein, 7 aa, Artificial Sequence
  Made in a lab
AASSLQS

SEQ ID NO: 42 - protein, 9 aa, Artificial Sequence
  Made in a lab
QQSYSVPIT

SEQ ID NO: 43 - protein, 11 aa, Artificial Sequence
  Made in a lab
RASQSMSNWLA

SEQ ID NO: 44 - protein, 7 aa, Artificial Sequence
  Made in a lab
DVFTLKS

SEQ ID NO: 45 - protein, 10 aa, Artificial Sequence
  Made in a lab
QQYSDYSGIT

SEQ ID NO: 46 - protein, 13 aa, Artificial Sequence
  Made in a lab
SGSSSNIGSKVVY

SEQ ID NO: 47 - protein, 7 aa, Artificial Sequence
  Made in a lab
RNDQRPS

SEQ ID NO: 48 - protein, 12 aa, Artificial Sequence
  Made in a lab
AAWDDSLSGRWV

SEQ ID NO: 49 - protein, 11 aa, Artificial Sequence
  Made in a lab
RASQSISSWLA

SEQ ID NO: 50 - protein, 7 aa, Artificial Sequence
  Made in a lab
KASGLES

SEQ ID NO: 51 - protein, 9 aa, Artificial Sequence
  Made in a lab
QQYNSYPFT

SEQ ID NO: 52 - protein, 11 aa, Artificial Sequence
  Made in a lab
QASRDISIYLN

SEQ ID NO: 53 - protein, 7 aa, Artificial Sequence
  Made in a lab
GASTLRS

SEQ ID NO: 54 - protein, 9 aa, Artificial Sequence
  Made in a lab
QKYNSAPYT

SEQ ID NO: 55 - protein, 11 aa, Artificial Sequence
  Made in a lab
RASQGISSWLA

SEQ ID NO: 56 - protein, 7 aa, Artificial Sequence
  Made in a lab
AASSLQS

SEQ ID NO: 57 - protein, 9 aa, Artificial Sequence
  Made in a lab
QQANSFPLT

SEQ ID NO: 58 - protein, 11 aa, Artificial Sequence
  Made in a lab
RASQSINTYLA

SEQ ID NO: 59 - protein, 7 aa, Artificial Sequence
  Made in a lab
DASNRAT

SEQ ID NO: 60 - protein, 9 aa, Artificial Sequence
  Made in a lab
QQYGRSRYT

SEQ ID NO: 61 - protein, 11 aa, Artificial Sequence
  Made in a lab
RASQGISNYLA

SEQ ID NO: 62 - protein, 7 aa, Artificial Sequence
  Made in a lab
AASTLQS

SEQ ID NO: 63 - protein, 9 aa, Artificial Sequence
  Made in a lab
QQSDNIPYT

SEQ ID NO: 64 - protein, 13 aa, Artificial Sequence
  Made in a lab
SGSSSNIGSNTVN

SEQ ID NO: 65 - protein, 7 aa, Artificial Sequence
  Made in a lab
SNNQRPS

SEQ ID NO: 66 - protein, 11 aa, Artificial Sequence
  Made in a lab
AAWDDSLNGYV

SEQ ID NO: 67 - protein, 11 aa, Artificial Sequence
  Made in a lab
RASQSISTYLN

SEQ ID NO: 68 - protein, 7 aa, Artificial Sequence
  Made in a lab
EATNLQS

SEQ ID NO: 69 - protein, 9 aa, Artificial Sequence
  Made in a lab
QQANNFPFT

SEQ ID NO: 70 - protein, 13 aa, Artificial Sequence
  Made in a lab
SGSSSNIGGNAVN

SEQ ID NO: 71 - protein, 7 aa, Artificial Sequence
  Made in a lab
SSNHRPS

SEQ ID NO: 72 - protein, 11 aa, Artificial Sequence
  Made in a lab
AAWDDSLNGPL

SEQ ID NO: 73 - protein, 10 aa, Artificial Sequence
  Made in a lab
SGEKLGDRYA

SEQ ID NO: 74 - protein, 7 aa, Artificial Sequence
  Made in a lab
QDTKRPS

SEQ ID NO: 75 - protein, 11 aa, Artificial Sequence
  Made in a lab
AAWDDSLKGVV

SEQ ID NO: 76 - protein, 13 aa, Artificial Sequence
  Made in a lab
SGRSSNIGSNSVN

SEQ ID NO: 77 - protein, 7 aa, Artificial Sequence
  Made in a lab
SNNHRPS

SEQ ID NO: 78 - protein, 11 aa, Artificial Sequence
  Made in a lab
AAWDDSLYQGV

SEQ ID NO: 79 - protein, 11 aa, Artificial Sequence
  Made in a lab
RASQSISSYLN

SEQ ID NO: 80 - protein, 7 aa, Artificial Sequence
  Made in a lab
AASSLQS

SEQ ID NO: 81 - protein, 9 aa, Artificial Sequence
  Made in a lab
QQYEYFPPT

SEQ ID NO: 82 - protein, 12 aa, Artificial Sequence
  Made in a lab
RASQSVSSSYLA

SEQ ID NO: 83 - protein, 7 aa, Artificial Sequence
  Made in a lab
GASSRAT

SEQ ID NO: 84 - protein, 9 aa, Artificial Sequence
  Made in a lab
QQYGSSSLT

SEQ ID NO: 85 - protein, 11 aa, Artificial Sequence
  Made in a lab
RSSQRISGYLN

SEQ ID NO: 86 - protein, 7 aa, Artificial Sequence
  Made in a lab
AASTLQS

SEQ ID NO: 87 - protein, 9 aa, Artificial Sequence
  Made in a lab
QQSYSPPFT

SEQ ID NO: 88 - protein, 11 aa, Artificial Sequence
  Made in a lab
RASQSISTWLA

SEQ ID NO: 89 - protein, 7 aa, Artificial Sequence
  Made in a lab
KASSLQS

SEQ ID NO: 90 - protein, 9 aa, Artificial Sequence
  Made in a lab
QQYKSYPFT

SEQ ID NO: 91 - protein, 11 aa, Artificial Sequence
  Made in a lab
QASQDIRSYLN

SEQ ID NO: 92 - protein, 7 aa, Artificial Sequence
  Made in a lab
DTVNLQT

SEQ ID NO: 93 - protein, 9 aa, Artificial Sequence
  Made in a lab
QQYEYLPIT

SEQ ID NO: 94 - protein, 11 aa, Artificial Sequence
  Made in a lab
RASQSISNYLN

SEQ ID NO: 95 - protein, 8 aa, Artificial Sequence
  Made in a lab
YAASSLHS

SEQ ID NO: 96 - protein, 9 aa, Artificial Sequence
  Made in a lab
QQSYSTSGT

SEQ ID NO: 97 - protein, 11 aa, Artificial Sequence
  Made in a lab
RASQSISSYLN

SEQ ID NO: 98 - protein, 7 aa, Artificial Sequence
  Made in a lab
AASSLQS

SEQ ID NO: 99 - protein, 9 aa, Artificial Sequence
  Made in a lab
QQSYSTPVT

SEQ ID NO: 100 - protein, 11 aa, Artificial Sequence
  Made in a lab
RASQSIDTYLN

SEQ ID NO: 101 - protein, 7 aa, Artificial Sequence
  Made in a lab
AASSLQS

SEQ ID NO: 102 - protein, 10 aa, Artificial Sequence
  Made in a lab
QQSYSSPGIT

SEQ ID NO: 103 - protein, 11 aa, Artificial Sequence
  Made in a lab
RASQSIGTYLN

SEQ ID NO: 104 - protein, 7 aa, Artificial Sequence
  Made in a lab
GTSSLQR

SEQ ID NO: 105 - protein, 9 aa, Artificial Sequence
  Made in a lab
QQSYTSPPT

SEQ ID NO: 106 - protein, 11 aa, Artificial Sequence
  Made in a lab
SGDQLGGKFTS

SEQ ID NO: 107 - protein, 7 aa, Artificial Sequence
  Made in a lab
QDTKRPS

SEQ ID NO: 108 - protein, 9 aa, Artificial Sequence
  Made in a lab
LVWDSNTYV

SEQ ID NO: 109 - protein, 11 aa, Artificial Sequence
  Made in a lab
GGNNIGSKSVH

SEQ ID NO: 110 - protein, 7 aa, Artificial Sequence
  Made in a lab
DDSDRPS

SEQ ID NO: 111 - protein, 11 aa, Artificial Sequence
  Made in a lab
QVWDSSGGLQV

SEQ ID NO: 112 - protein, 11 aa, Artificial Sequence
  Made in a lab
RASQSISSWLA

SEQ ID NO: 113 - protein, 7 aa, Artificial Sequence
  Made in a lab
KASNLES

SEQ ID NO: 114 - protein, 9 aa, Artificial Sequence
  Made in a lab
QQYNAYPFI

SEQ ID NO: 115 - protein, 11 aa, Artificial Sequence
  Made in a lab
RASQSISSWLA

SEQ ID NO: 116 - protein, 7 aa, Artificial Sequence
  Made in a lab
KASSLES

SEQ ID NO: 117 - protein, 8 aa, Artificial Sequence
  Made in a lab
QQYKSYWT

SEQ ID NO: 118 - protein, 13 aa, Artificial Sequence
  Made in a lab
SGSPSNIGSNSVN

SEQ ID NO: 119 - protein, 7 aa, Artificial Sequence
  Made in a lab
YDDLVPS

SEQ ID NO: 120 - protein, 11 aa, Artificial Sequence
  Made in a lab
AAWDDRLNGWV

SEQ ID NO: 121 - protein, 11 aa, Artificial Sequence
  Made in a lab
RASQGISSWLA

SEQ ID NO: 122 - protein, 7 aa, Artificial Sequence
  Made in a lab
AASSLQS

SEQ ID NO: 123 - protein, 9 aa, Artificial Sequence
  Made in a lab
QQANSFPLT

SEQ ID NO: 124 - protein, 11 aa, Artificial Sequence
  Made in a lab
RASQSISSYLN

SEQ ID NO: 125 - protein, 7 aa, Artificial Sequence
  Made in a lab
AASSLQS

SEQ ID NO: 126 - protein, 9 aa, Artificial Sequence
  Made in a lab
QQSYSTPYT

SEQ ID NO: 127 - protein, 12 aa, Artificial Sequence
  Made in a lab
RASQSVSSSYLA

SEQ ID NO: 128 - protein, 7 aa, Artificial Sequence
  Made in a lab
GASNRVT

SEQ ID NO: 129 - protein, 8 aa, Artificial Sequence
  Made in a lab
HQYGSQYT

SEQ ID NO: 130 - protein, 11 aa, Artificial Sequence
  Made in a lab
QASQHISNFLN

SEQ ID NO: 131 - protein, 7 aa, Artificial Sequence
  Made in a lab
DTSNLRS

SEQ ID NO: 132 - protein, 9 aa, Artificial Sequence
  Made in a lab
QQYDNFPLT

SEQ ID NO: 133 - protein, 13 aa, Artificial Sequence
  Made in a lab
SGSTSNIGNNAVI

SEQ ID NO: 134 - protein, 7 aa, Artificial Sequence
  Made in a lab
YDDLLPS

SEQ ID NO: 135 - protein, 11 aa, Artificial Sequence
  Made in a lab
ASWDDNLNGPL

SEQ ID NO: 136 - protein, 11 aa, Artificial Sequence
  Made in a lab
RASQSISSWLA

SEQ ID NO: 137 - protein, 7 aa, Artificial Sequence
  Made in a lab
QASTLES

SEQ ID NO: 138 - protein, 9 aa, Artificial Sequence
  Made in a lab
QQANSFPIT

SEQ ID NO: 139 - protein, 11 aa, Artificial Sequence
  Made in a lab
QASQDITNYLN

SEQ ID NO: 140 - protein, 7 aa, Artificial Sequence
  Made in a lab
DASNLQT

SEQ ID NO: 141 - protein, 9 aa, Artificial Sequence
  Made in a lab
QQYDTLHPS

SEQ ID NO: 142 - protein, 14 aa, Artificial Sequence
  Made in a lab
TGTSSDVGVYDSVS

SEQ ID NO: 143 - protein, 7 aa, Artificial Sequence
  Made in a lab
DVSDRPS

SEQ ID NO: 144 - protein, 10 aa, Artificial Sequence
  Made in a lab
GSYRASSSYV

SEQ ID NO: 145 - protein, 11 aa, Artificial Sequence
  Made in a lab
RASQNIYHYLH

SEQ ID NO: 146 - protein, 7 aa, Artificial Sequence
  Made in a lab
AASTLES

SEQ ID NO: 147 - protein, 9 aa, Artificial Sequence
  Made in a lab
QQTYDTPLT

SEQ ID NO: 148 - protein, 11 aa, Artificial Sequence
  Made in a lab
SGDKLGEKFAS

SEQ ID NO: 149 - protein, 7 aa, Artificial Sequence
  Made in a lab
QDSKRPS

SEQ ID NO: 150 - protein, 10 aa, Artificial Sequence
  Made in a lab
QAWGGSTAYV

SEQ ID NO: 151 - protein, 11 aa, Artificial Sequence
  Made in a lab
RASQSVTTYLA

SEQ ID NO: 152 - protein, 7 aa, Artificial Sequence
  Made in a lab
DASNRAT

SEQ ID NO: 153 - protein, 11 aa, Artificial Sequence
  Made in a lab
QQRSNWPPSIT

SEQ ID NO: 154 - protein, 14 aa, Artificial Sequence
  Made in a lab
TGTSSDVGSYNLVS

SEQ ID NO: 155 - protein, 7 aa, Artificial Sequence
  Made in a lab
EGSKRPS

SEQ ID NO: 156 - protein, 10 aa, Artificial Sequence
  Made in a lab
CSYAGRSTYV

SEQ ID NO: 157 - protein, 11 aa, Artificial Sequence
  Made in a lab
SGNRLEDKYVH

SEQ ID NO: 158 - protein, 7 aa, Artificial Sequence
  Made in a lab
QDNKRPS

SEQ ID NO: 159 - protein, 10 aa, Artificial Sequence
  Made in a lab
QAWDSSTREV

SEQ ID NO: 160 - protein, 11 aa, Artificial Sequence
  Made in a lab
RASQGVSDWLA

SEQ ID NO: 161 - protein, 7 aa, Artificial Sequence
  Made in a lab
GASSLQT

SEQ ID NO: 162 - protein, 9 aa, Artificial Sequence
  Made in a lab
QQAHSFPFT

SEQ ID NO: 163 - protein, 11 aa, Artificial Sequence
  Made in a lab
RASQNINTHLA

SEQ ID NO: 164 - protein, 7 aa, Artificial Sequence
  Made in a lab
GASTRAT

SEQ ID NO: 165 - protein, 10 aa, Artificial Sequence
  Made in a lab
QQYGSSLTWT

SEQ ID NO: 166 - protein, 12 aa, Artificial Sequence
  Made in a lab
RASQSVSGNYLA

SEQ ID NO: 167 - protein, 7 aa, Artificial Sequence
  Made in a lab
GASSRAT

SEQ ID NO: 168 - protein, 9 aa, Artificial Sequence
  Made in a lab
LQHNSYPLT

SEQ ID NO: 169 - protein, 11 aa, Artificial Sequence
  Made in a lab
GGINIGSKSVH

SEQ ID NO: 170 - protein, 8 aa, Artificial Sequence
  Made in a lab
YFDSDRPS

SEQ ID NO: 171 - protein, 11 aa, Artificial Sequence
  Made in a lab
QVWDSRSDQYV

SEQ ID NO: 172 - protein, 11 aa, Artificial Sequence
  Made in a lab
RASQSISSWLA

SEQ ID NO: 173 - protein, 7 aa, Artificial Sequence
  Made in a lab
KASTLES

SEQ ID NO: 174 - protein, 9 aa, Artificial Sequence
  Made in a lab
QQYNSYSLT

SEQ ID NO: 175 - protein, 11 aa, Artificial Sequence
  Made in a lab
RASQSISSYLN

SEQ ID NO: 176 - protein, 7 aa, Artificial Sequence
  Made in a lab
AASSLQS

SEQ ID NO: 177 - protein, 9 aa, Artificial Sequence
  Made in a lab
QQSYSTPLT

SEQ ID NO: 178 - protein, 11 aa, Artificial Sequence
  Made in a lab
RASQSINNYLN

SEQ ID NO: 179 - protein, 7 aa, Artificial Sequence
  Made in a lab
TASTLHS

SEQ ID NO: 180 - protein, 9 aa, Artificial Sequence
  Made in a lab
QQSYATLRT

SEQ ID NO: 181 - protein, 14 aa, Artificial Sequence
  Made in a lab
SGSSSDVGHYDYVS

SEQ ID NO: 182 - protein, 7 aa, Artificial Sequence
  Made in a lab
DVSNRPS

SEQ ID NO: 183 - protein, 10 aa, Artificial Sequence
  Made in a lab
SSYTSSGTRV

SEQ ID NO: 184 - protein, 11 aa, Artificial Sequence
  Made in a lab
RASQSVNNWVA

SEQ ID NO: 185 - protein, 7 aa, Artificial Sequence
  Made in a lab
AASSLQS

SEQ ID NO: 186 - protein, 9 aa, Artificial Sequence
  Made in a lab
QQSYSMPLT

SEQ ID NO: 187 - protein, 5 aa, Artificial Sequence
  Made in a lab
KYSMH

SEQ ID NO: 188 - protein, 17 aa, Artificial Sequence
  Made in a lab
SIWPSGGWTTYADSVKG

SEQ ID NO: 189 - protein, 9 aa, Artificial Sequence
  Made in a lab
GGYYYALDY

SEQ ID NO: 190 - protein, 5 aa, Artificial Sequence
  Made in a lab
FYQMH

SEQ ID NO: 191 - protein, 17 aa, Artificial Sequence
  Made in a lab
GIYPSGGYTKYADSVKG

SEQ ID NO: 192 - protein, 13 aa, Artificial Sequence
  Made in a lab
DLGYGSSWYYFDY

SEQ ID NO: 193 - protein, 5 aa, Artificial Sequence
  Made in a lab
KYSMH

SEQ ID NO: 194 - protein, 16 aa, Artificial Sequence
  Made in a lab
GISPSGYTRYADSVKG

SEQ ID NO: 195 - protein, 9 aa, Artificial Sequence
  Made in a lab
DQLLEAFDI

SEQ ID NO: 196 - protein, 5 aa, Artificial Sequence
  Made in a lab
EYPMN

SEQ ID NO: 197 - protein, 17 aa, Artificial Sequence
  Made in a lab
GIYSSGGLTKYADSVKG

SEQ ID NO: 198 - protein, 10 aa, Artificial Sequence
  Made in a lab
DHYGDYAFDY

SEQ ID NO: 199 - protein, 5 aa, Artificial Sequence
  Made in a lab
LYEMV

SEQ ID NO: 200 - protein, 17 aa, Artificial Sequence
  Made in a lab
GIWPSGGWTQYADSVKG

SEQ ID NO: 201 - protein, 17 aa, Artificial Sequence
  Made in a lab
DLAVAGWDYYYYYGMDV

SEQ ID NO: 202 - protein, 5 aa, Artificial Sequence
  Made in a lab
QYNMI

SEQ ID NO: 203 - protein, 16 aa, Artificial Sequence
  Made in a lab
WISSSGGTEYADSVKG

SEQ ID NO: 204 - protein, 17 aa, Artificial Sequence
  Made in a lab
DRGYGSGSYGAYDAFDI

SEQ ID NO: 205 - protein, 5 aa, Artificial Sequence
  Made in a lab
HYSMG

SEQ ID NO: 206 - protein, 17 aa, Artificial Sequence
  Made in a lab
SIYPSGGSTLYADSVKG

SEQ ID NO: 207 - protein, 9 aa, Artificial Sequence
  Made in a lab
WGVGATFDY

SEQ ID NO: 208 - protein, 5 aa, Artificial Sequence
  Made in a lab
HYMMA

SEQ ID NO: 209 - protein, 17 aa, Artificial Sequence
  Made in a lab
YIYPSGGWTSYADSVKG

SEQ ID NO: 210 - protein, 9 aa, Artificial Sequence
  Made in a lab
FDYTIGFDF

SEQ ID NO: 211 - protein, 5 aa, Artificial Sequence
  Made in a lab
HYGMT

SEQ ID NO: 212 - protein, 17 aa, Artificial Sequence
  Made in a lab
GIRSSGGVTNYADSVKG

SEQ ID NO: 213 - protein, 11 aa, Artificial Sequence
  Made in a lab
EGSGWSKAFDI

SEQ ID NO: 214 - protein, 5 aa, Artificial Sequence
  Made in a lab
HYNMR

SEQ ID NO: 215 - protein, 17 aa, Artificial Sequence
  Made in a lab
SISPSGGFTGYADSVKG

SEQ ID NO: 216 - protein, 18 aa, Artificial Sequence
  Made in a lab
GINYYDSSGYYPPVGMDV

SEQ ID NO: 217 - protein, 5 aa, Artificial Sequence
  Made in a lab
WYNML

SEQ ID NO: 218 - protein, 17 aa, Artificial Sequence
  Made in a lab
SISPSGGYTVYADSVKG

SEQ ID NO: 219 - protein, 14 aa, Artificial Sequence
  Made in a lab
DRGGSPFRPDAFDI

SEQ ID NO: 220 - protein, 5 aa, Artificial Sequence
  Made in a lab
EYDML

SEQ ID NO: 221 - protein, 17 aa, Artificial Sequence
  Made in a lab
SIWPSGGFTQYADSVKG

SEQ ID NO: 222 - protein, 16 aa, Artificial Sequence
  Made in a lab
NYYDFWSGPYYYGMDV

SEQ ID NO: 223 - protein, 5 aa, Artificial Sequence
  Made in a lab
GYVMG

SEQ ID NO: 224 - protein, 17 aa, Artificial Sequence
  Made in a lab
SISPSGGYTLYADSVKG

SEQ ID NO: 225 - protein, 13 aa, Artificial Sequence
  Made in a lab
AFSTRWYWGAFDI

SEQ ID NO: 226 - protein, 5 aa, Artificial Sequence
  Made in a lab
IYHMN

SEQ ID NO: 227 - protein, 17 aa, Artificial Sequence
  Made in a lab
GISSSGGRTNYADSVKG

SEQ ID NO: 228 - protein, 16 aa, Artificial Sequence
  Made in a lab
SYRAAGWVDYYYGMDV

SEQ ID NO: 229 - protein, 5 aa, Artificial Sequence
  Made in a lab
LYDMH

SEQ ID NO: 230 - protein, 17 aa, Artificial Sequence
  Made in a lab
RIYSSGGTTQYADSVKG

SEQ ID NO: 231 - protein, 12 aa, Artificial Sequence
  Made in a lab
DPGYGSHHSFDY

SEQ ID NO: 232 - protein, 5 aa, Artificial Sequence
  Made in a lab
SYYMT

SEQ ID NO: 233 - protein, 17 aa, Artificial Sequence
  Made in a lab
GIYSSGGPTQYADSVKG

SEQ ID NO: 234 - protein, 8 aa, Artificial Sequence
  Made in a lab
AGGDAFDI

SEQ ID NO: 235 - protein, 5 aa, Artificial Sequence
  Made in a lab
SYEMF

SEQ ID NO: 236 - protein, 17 aa, Artificial Sequence
  Made in a lab
SISPSGGYTYYADSVKG

SEQ ID NO: 237 - protein, 9 aa, Artificial Sequence
  Made in a lab
MTTSGFHLI

SEQ ID NO: 238 - protein, 5 aa, Artificial Sequence
  Made in a lab
DYPMQ

SEQ ID NO: 239 - protein, 17 aa, Artificial Sequence
  Made in a lab
WIGPSGGWTVYADSVKG

SEQ ID NO: 240 - protein, 8 aa, Artificial Sequence
  Made in a lab
DDGIAGFL

SEQ ID NO: 241 - protein, 5 aa, Artificial Sequence
  Made in a lab
WYLMH

SEQ ID NO: 242 - protein, 17 aa, Artificial Sequence
  Made in a lab
GIWPSGGHTLYADSVKG

SEQ ID NO: 243 - protein, 16 aa, Artificial Sequence
  Made in a lab
EPLLWFGELSYNWFDP

SEQ ID NO: 244 - protein, 6 aa, Artificial Sequence
  Made in a lab
YYENMA

SEQ ID NO: 245 - protein, 17 aa, Artificial Sequence
  Made in a lab
GIYPSGGLTYYADSVKG

SEQ ID NO: 246 - protein, 13 aa, Artificial Sequence
  Made in a lab
SRRYYDSSDAFDI

SEQ ID NO: 247 - protein, 5 aa, Artificial Sequence
  Made in a lab
TYVMY

SEQ ID NO: 248 - protein, 17 aa, Artificial Sequence
  Made in a lab
GIGPSGGNTFYADSVKG

SEQ ID NO: 249 - protein, 14 aa, Artificial Sequence
  Made in a lab
DPGDFWSGYYGMDV

SEQ ID NO: 250 - protein, 5 aa, Artificial Sequence
  Made in a lab
FYGMA

SEQ ID NO: 251 - protein, 17 aa, Artificial Sequence
  Made in a lab
SIYPSGGYTDYADSVKG

SEQ ID NO: 252 - protein, 13 aa, Artificial Sequence
  Made in a lab
SAGGWIGGGAFDI

SEQ ID NO: 253 - protein, 5 aa, Artificial Sequence
  Made in a lab
KYPMM

SEQ ID NO: 254 - protein, 17 aa, Artificial Sequence
  Made in a lab
YIYPSGGKTAYADSVKG

SEQ ID NO: 255 - protein, 17 aa, Artificial Sequence
  Made in a lab
YGSGSYYLYYYYYYMDV

SEQ ID NO: 256 - protein, 5 aa, Artificial Sequence
  Made in a lab
WYSMW

SEQ ID NO: 257 - protein, 17 aa, Artificial Sequence
  Made in a lab
YIVPSGGETIYADSVKG

SEQ ID NO: 258 - protein, 16 aa, Artificial Sequence
  Made in a lab
NLGEGFWSDYYPPLDY

SEQ ID NO: 259 - protein, 5 aa, Artificial Sequence
  Made in a lab
HYGMH

SEQ ID NO: 260 - protein, 15 aa, Artificial Sequence
  Made in a lab
SIYPGMTQYADSVKG

SEQ ID NO: 261 - protein, 11 aa, Artificial Sequence
  Made in a lab
DRGSGIDAFDI

SEQ ID NO: 262 - protein, 5 aa, Artificial Sequence
  Made in a lab
WYDML

SEQ ID NO: 263 - protein, 17 aa, Artificial Sequence
  Made in a lab
VISPSGGRTFYADSVKG

SEQ ID NO: 264 - protein, 16 aa, Artificial Sequence
  Made in a lab
TRSMYSSSWYGAPPTH

SEQ ID NO: 265 - protein, 5 aa, Artificial Sequence
  Made in a lab
WYKMH

SEQ ID NO: 266 - protein, 17 aa, Artificial Sequence
  Made in a lab
GISSSGGLTKYADSVKG

SEQ ID NO: 267 - protein, 11 aa, Artificial Sequence
  Made in a lab
ERRGDGGAFDI

SEQ ID NO: 268 - protein, 5 aa, Artificial Sequence
  Made in a lab
EYTMY

SEQ ID NO: 269 - protein, 17 aa, Artificial Sequence
  Made in a lab
YISPSGGTTNYADSVKG

SEQ ID NO: 270 - protein, 13 aa, Artificial Sequence
  Made in a lab
GAWGDIYYYGMDV

SEQ ID NO: 271 - protein, 5 aa, Artificial Sequence
  Made in a lab
SYWMH

SEQ ID NO: 272 - protein, 17 aa, Artificial Sequence
  Made in a lab
SIYPSGGSTEYADSVKG

SEQ ID NO: 273 - protein, 6 aa, Artificial Sequence
  Made in a lab
GIWFDP

SEQ ID NO: 274 - protein, 5 aa, Artificial Sequence
  Made in a lab
RYGMM

SEQ ID NO: 275 - protein, 17 aa, Artificial Sequence
  Made in a lab
YISSSGGFTRYADSVKG

SEQ ID NO: 276 - protein, 12 aa, Artificial Sequence
  Made in a lab
VGGYSYGPHFDF

SEQ ID NO: 277 - protein, 5 aa, Artificial Sequence
  Made in a lab
WYHMI

SEQ ID NO: 278 - protein, 17 aa, Artificial Sequence
  Made in a lab
WISPSGGFTKYADSVKG

SEQ ID NO: 279 - protein, 14 aa, Artificial Sequence
  Made in a lab
YDSRAAAGTNAFDI

SEQ ID NO: 280 - protein, 5 aa, Artificial Sequence
  Made in a lab
PYKMV

SEQ ID NO: 281 - protein, 17 aa, Artificial Sequence
  Made in a lab
GISPSGGYTYYADSVKG

SEQ ID NO: 282 - protein, 14 aa, Artificial Sequence
  Made in a lab
GGYGWSYYYYGMDV

SEQ ID NO: 283 - protein, 5 aa, Artificial Sequence
  Made in a lab
NYWMY

SEQ ID NO: 284 - protein, 17 aa, Artificial Sequence
  Made in a lab
VISSSGGHTFYADSVKG

SEQ ID NO: 285 - protein, 9 aa, Artificial Sequence
  Made in a lab
DYEGGSNDY

SEQ ID NO: 286 - protein, 5 aa, Artificial Sequence
  Made in a lab
NYAMS

SEQ ID NO: 287 - protein, 17 aa, Artificial Sequence
  Made in a lab
SIYSSGGYTAYADSVKG

SEQ ID NO: 288 - protein, 11 aa, Artificial Sequence
  Made in a lab
VPHVFRGELDY

SEQ ID NO: 289 - protein, 5 aa, Artificial Sequence
  Made in a lab
FYTMW

SEQ ID NO: 290 - protein, 17 aa, Artificial Sequence
  Made in a lab
SIYPSGGQTLYADSVKG

SEQ ID NO: 291 - protein, 13 aa, Artificial Sequence
  Made in a lab
PDSYGYLYYGMDV

SEQ ID NO: 292 - protein, 5 aa, Artificial Sequence
  Made in a lab
WYPME

SEQ ID NO: 293 - protein, 17 aa, Artificial Sequence
  Made in a lab
GIGPSGGQTTYADSVKG

SEQ ID NO: 294 - protein, 6 aa, Artificial Sequence
  Made in a lab
GSYSFI

SEQ ID NO: 295 - protein, 5 aa, Artificial Sequence
  Made in a lab
LYKMA

SEQ ID NO: 296 - protein, 17 aa, Artificial Sequence
  Made in a lab
VIGSSGGRTPYADSVKG

SEQ ID NO: 297 - protein, 16 aa, Artificial Sequence
  Made in a lab
APLSGWFGQAHDAFDI

SEQ ID NO: 298 - protein, 5 aa, Artificial Sequence
  Made in a lab
TYEMN

SEQ ID NO: 299 - protein, 17 aa, Artificial Sequence
  Made in a lab
GIVPSGGVTYYADSVKG

SEQ ID NO: 300 - protein, 13 aa, Artificial Sequence
  Made in a lab
GPYSYGHDYGMDV

SEQ ID NO: 301 - protein, 5 aa, Artificial Sequence
  Made in a lab
VYPMH

SEQ ID NO: 302 - protein, 17 aa, Artificial Sequence
  Made in a lab
SIYSSGGFTMYADSVKG

SEQ ID NO: 303 - protein, 9 aa, Artificial Sequence
  Made in a lab
EGVADAFDI

SEQ ID NO: 304 - protein, 5 aa, Artificial Sequence
  Made in a lab
SYTMH

SEQ ID NO: 305 - protein, 17 aa, Artificial Sequence
  Made in a lab
SISPSGGMTFYADSVKG

SEQ ID NO: 306 - protein, 11 aa, Artificial Sequence
  Made in a lab
TYDFWSGYFDY

SEQ ID NO: 307 - protein, 5 aa, Artificial Sequence
  Made in a lab
TYEMG

SEQ ID NO: 308 - protein, 17 aa, Artificial Sequence
  Made in a lab
VIWSSGGHTWYADSVKG

SEQ ID NO: 309 - protein, 16 aa, Artificial Sequence
  Made in a lab
SNQGDFWSGYPFAFDI

SEQ ID NO: 310 - protein, 5 aa, Artificial Sequence
  Made in a lab
NYNMY

SEQ ID NO: 311 - protein, 17 aa, Artificial Sequence
  Made in a lab
YISPSGGMTKYADSVKG

SEQ ID NO: 312 - protein, 11 aa, Artificial Sequence
  Made in a lab
RGVLGYYGMDV

SEQ ID NO: 313 - protein, 5 aa, Artificial Sequence
  Made in a lab
LYSMN

SEQ ID NO: 314 - protein, 17 aa, Artificial Sequence
  Made in a lab
SISSSGGATLYADSVKG

SEQ ID NO: 315 - protein, 14 aa, Artificial Sequence
  Made in a lab
DLISSGYYPDAFDI

SEQ ID NO: 316 - protein, 5 aa, Artificial Sequence
  Made in a lab
QYMMH

SEQ ID NO: 317 - protein, 17 aa, Artificial Sequence
  Made in a lab
SISSSGGWTSYADSVKG

SEQ ID NO: 318 - protein, 19 aa, Artificial Sequence
  Made in a lab
PLALGYDFWSGYQAAGFDY

SEQ ID NO: 319 - protein, 5 aa, Artificial Sequence
  Made in a lab
WYSMV

SEQ ID NO: 320 - protein, 17 aa, Artificial Sequence
  Made in a lab
SIVPSGGLTWYADSVKG

SEQ ID NO: 321 - protein, 10 aa, Artificial Sequence
  Made in a lab
DVYGDYYFDN

SEQ ID NO: 322 - protein, 5 aa, Artificial Sequence
  Made in a lab
YYNMT

SEQ ID NO: 323 - protein, 17 aa, Artificial Sequence
  Made in a lab
SISPSGGRTGYADSVKG

SEQ ID NO: 324 - protein, 10 aa, Artificial Sequence
  Made in a lab
VSGFYAAFDY

SEQ ID NO: 325 - protein, 5 aa, Artificial Sequence
  Made in a lab
WYWMT

SEQ ID NO: 326 - protein, 17 aa, Artificial Sequence
  Made in a lab
SISPSGGHTSYADSVKG

SEQ ID NO: 327 - protein, 11 aa, Artificial Sequence
  Made in a lab
SSGWYEDYFDY

SEQ ID NO: 328 - protein, 5 aa, Artificial Sequence
  Made in a lab
PYEMA

SEQ ID NO: 329 - protein, 17 aa, Artificial Sequence
  Made in a lab
VIGPSGGYTEYADSVKG

SEQ ID NO: 330 - protein, 14 aa, Artificial Sequence
  Made in a lab
GYDFWSGYYDAFDI

SEQ ID NO: 331 - protein, 5 aa, Artificial Sequence
  Made in a lab
SYFMG

SEQ ID NO: 332 - protein, 17 aa, Artificial Sequence
  Made in a lab
SIWPSGGNTTYADSVKG

SEQ ID NO: 333 - protein, 10 aa, Artificial Sequence
  Made in a lab
HVGWGRYFDY

SEQ ID NO: 334 - protein, 5 aa, Artificial Sequence
  Made in a lab
AYRMI

SEQ ID NO: 335 - protein, 17 aa, Artificial Sequence
  Made in a lab
YISSSGGRTDYADSVKG

SEQ ID NO: 336 - protein, 15 aa, Artificial Sequence
  Made in a lab
GGLRYFDWLAPSMDY

SEQ ID NO: 337 - protein, 5 aa, Artificial Sequence
  Made in a lab
AYSMV

SEQ ID NO: 338 - protein, 17 aa, Artificial Sequence
  Made in a lab
YIYPSGGITTYADSVKG

SEQ ID NO: 339 - protein, 7 aa, Artificial Sequence
  Made in a lab
EGQVFDI

SEQ ID NO: 340 - protein, 5 aa, Artificial Sequence
  Made in a lab
NYAMG

SEQ ID NO: 341 - protein, 17 aa, Artificial Sequence
  Made in a lab
RIVPSGGMTSYADSVKG

SEQ ID NO: 342 - protein, 9 aa, Artificial Sequence
  Made in a lab
DDFWSGMDV

SEQ ID NO: 343 - protein, 5 aa, Artificial Sequence
  Made in a lab
LYNMQ

SEQ ID NO: 344 - protein, 17 aa, Artificial Sequence
  Made in a lab
GISLSGGKTFYADSVKG

SEQ ID NO: 345 - protein, 12 aa, Artificial Sequence
  Made in a lab
DYGVATLDAFDI

SEQ ID NO: 346 - protein, 5 aa, Artificial Sequence
  Made in a lab
AYTMH

SEQ ID NO: 347 - protein, 17 aa, Artificial Sequence
  Made in a lab
SIYPSGGTTPYADSVKG

SEQ ID NO: 348 - protein, 18 aa, Artificial Sequence
  Made in a lab
VVNIDFWSGYNMRSAFDI

SEQ ID NO: 349 - protein, 5 aa, Artificial Sequence
  Made in a lab
DYLMG

SEQ ID NO: 350 - protein, 17 aa, Artificial Sequence
  Made in a lab
VISSSGGPTAYADSVKG

SEQ ID NO: 351 - protein, 13 aa, Artificial Sequence
  Made in a lab
VGLDYGILGAFDI

SEQ ID NO: 352 - protein, 5 aa, Artificial Sequence
  Made in a lab
EYGMS

SEQ ID NO: 353 - protein, 17 aa, Artificial Sequence
  Made in a lab
SIRSSGGWTKYADSVKG

SEQ ID NO: 354 - protein, 11 aa, Artificial Sequence
  Made in a lab
GLGATSGEFDY

SEQ ID NO: 355 - protein, 5 aa, Artificial Sequence
  Made in a lab
KYDMW

SEQ ID NO: 356 - protein, 17 aa, Artificial Sequence
  Made in a lab
SIWPSGGWTSYADSVKG

SEQ ID NO: 357 - protein, 12 aa, Artificial Sequence
  Made in a lab
SRGSPWYGDFDH

SEQ ID NO: 358 - protein, 5 aa, Artificial Sequence
  Made in a lab
MYTMH

SEQ ID NO: 359 - protein, 17 aa, Artificial Sequence
  Made in a lab
WISPSGGWTKYADSVKG

SEQ ID NO: 360 - protein, 9 aa, Artificial Sequence
  Made in a lab
SPWGGPFDY

SEQ ID NO: 361 - protein, 5 aa, Artificial Sequence
  Made in a lab
QYFMM

SEQ ID NO: 362 - protein, 17 aa, Artificial Sequence
  Made in a lab
SISPSGGYTQYADSVKG

SEQ ID NO: 363 - protein, 14 aa, Artificial Sequence
  Made in a lab
TGDYTRYYSYGMDV

SEQ ID NO: 364 - protein, 5 aa, Artificial Sequence
  Made in a lab
GYSMA

SEQ ID NO: 365 - protein, 17 aa, Artificial Sequence
  Made in a lab
GIYSSGGWTWYADSVKG

SEQ ID NO: 366 - protein, 15 aa, Artificial Sequence
  Made in a lab
DQSFSYDSSLDAFDI

SEQ ID NO: 367 - protein, 5 aa, Artificial Sequence
  Made in a lab
LYGMH

SEQ ID NO: 368 - protein, 17 aa, Artificial Sequence
  Made in a lab
SIVPSGGLTRYADSVKG

SEQ ID NO: 369 - protein, 15 aa, Artificial Sequence
  Made in a lab
LAYYDFWSGRDAFDI

SEQ ID NO: 370 - protein, 5 aa, Artificial Sequence
  Made in a lab
HYGMD

SEQ ID NO: 371 - protein, 17 aa, Artificial Sequence
  Made in a lab
SIYSSGGYTLYADSVKG

SEQ ID NO: 372 - protein, 9 aa, Artificial Sequence
  Made in a lab
RTWADAFDV

SEQ ID NO: 373 - protein, 105 aa, Artificial Sequence
  Made in a lab
QSELTQPPSVSVSPGQTATITCSGEKLGDIYASWYQQKPGQSPVLVIYQDTKRPSGIPVRFSGSNSGNTATLTISGTQAMDEADYYCQAWDSSTVFGGGTKLTVL

SEQ ID NO: 374 - protein, 110 aa, Artificial Sequence
  Made in a lab
QSALTQPASVSGSPGQSITISCSGTDSDVGGYNHVSWYQQHPGKAPKLIIYDVDHRPSGISNRFSGSKSGNTASLTISGLQAEDEADYYCSSYRSGSTYVFGTATKVTVL

SEQ ID NO: 375 - protein, 108 aa, Artificial Sequence
  Made in a lab
QDIQMTQSPLSLSASVGDRVTITCRASQSISSYLNWYQQKPGEAPKLLIYAASALQSGVPSRFSGSGLGTVFTLTITSLQPEDSATYYCQQSYSPPVTFGGGTKVDIK

SEQ ID NO: 376 - protein, 107 aa, Artificial Sequence
  Made in a lab
QDIQMTQSPSTLSASVGDRVTITCRASQSISSWLAWYQQKPGKAPKLLIYKASSLESGVPSRFSGSGSGTEFTLTISSLQPDDFATYYCQQYNSYWTFGQGTKVEIK

SEQ ID NO: 377 - protein, 109 aa, Artificial Sequence
  Made in a lab
QDIQMTQSPATLSLSPGERATLSCRASQSVSSYLAWYQQKPGQAPRLLIYDASNRATGIPARFSGSGSGTDFTLTISSLEPEDSAVYYCQQRSNWPPFTFGGGTKVEIK

SEQ ID NO: 378 - protein, 108 aa, Artificial Sequence
  Made in a lab
QDIQMTQSPSSLSASVGDRVTITCQTSQSIDRYLNWYQQKAGEAPKLLIYAASSLQSGVPSRFSGSGSGTDFTLTISSLQPEDFASYYCQQSYRTPRTFGQGTKVEIK

SEQ ID NO: 379 - protein, 110 aa, Artificial Sequence
  Made in a lab
QDIQMTQSPGTLSLSPGERATLSCRASQSVSSSYLAWYQQKPGQAPRLLIYGASSRATGIPDRFSGSGSGTDFTLTISRLEPEDFAVYYCQQYGSSPLWTFGQGTKVEIK

SEQ ID NO: 380 - protein, 108 aa, Artificial Sequence
  Made in a lab
QDIQMTQSPSSLSASVGDRVTITCRASQSISSYLNWYQQKPGKAPKLLIYAASSLQSGVPSRFSGSGSGTDFTLTISSLQPEDFATYYCQQSYSTPRTFGQGTKVEIK

SEQ ID NO: 381 - protein, 110 aa, Artificial Sequence
  Made in a lab
QDIQMTQSPATLSLSPGERATLSCRASQSISTSLAWYQQIPGQAPRLLMYDASKRASGIPARFSGSGSGTDFTLTISSLEPEDSAVYYCQLRINWPPEFTFGPGTKVEIK

SEQ ID NO: 382 - protein, 108 aa, Artificial Sequence
  Made in a lab
QDIQMTQSPSSVSASVGDRVTITCRASRGVITWLNWYQQKPGRVPSPLIFGASTLQTGVPSRFSGSGSGTDFTLTINDLQPEDFATYYCQQTHSFPLTFGGGTKVEIK

SEQ ID NO: 383 - protein, 106 aa, Artificial Sequence
  Made in a lab
QSALTQPPSVSVSPGQTARITCSGDKLGEKYVSWYQQRSGQSPLLVISQDTKRPSGIPERFSGSNSGNTATLTISGAQAADEADYYCQTWDSDTYVFGSGSTVTVL

SEQ ID NO: 384 - protein, 108 aa, Artificial Sequence
  Made in a lab
QDIQMTQSPSSLSASVGDRVTITCRASQSISSYLNWYQQKPGKAPKLLIYAASSLQSGVPSRFSGSGSGTDFTLTISSLQPEDFATYYCQQSYSTLWTFGQGTKVEIK

SEQ ID NO: 385 - protein, 108 aa, Artificial Sequence
  Made in a lab
QDIQMTQSPATLSLSPGERATLSCRASQSVRSYLAWYQQKPGQAPRLLIYDASNRATGIPARFSGSGSGTDFTLTITSLQPEDIATYYCQQYDNLPLTFGGGTKVEIK

SEQ ID NO: 386 - protein, 108 aa, Artificial Sequence
  Made in a lab
QDIQMTQSPSSLSAFVGDRVTITCRTSQSVATYVNWYQQKPGEGPKLLIYAASSLQSGVPSRFRGSGSGTDFTLTIGSLQPEDFATYYCQQSYSVPITFGQGTRLDIK

SEQ ID NO: 387 - protein, 109 aa, Artificial Sequence
  Made in a lab
QDIQMTQSPSTLSASVGDRVTITCRASQSMSNWLAWYQQKPGKAPKLLIYDVFTLKSGVPSRFSGSRSGTEFTLTISSLQPDDFATYYCQQYSDYSGITFGPGTEVDIR

SEQ ID NO: 388 - protein, 111 aa, Artificial Sequence
  Made in a lab
QSVLTQPPSASGTPGQRVTISCSGSSSNIGSKVVYWYQQVPGTAPKLLIYRNDQRPSGVPDRFSGSKSGTSASLAISGLRSEDEADYYCAAWDDSLSGRWVFGGGTKLTVL

SEQ ID NO: 389 - protein, 108 aa, Artificial Sequence
  Made in a lab
QDIQMTQSPATLSASVGDRVTITCRASQSISSWLAWYQQKPGKAPKLLIYKASGLESGVPSRFSGSGSGTEFTLTISSLQPDDFATYYCQQYNSYPFTFGPGTKVDIK

SEQ ID NO: 390 - protein, 108 aa, Artificial Sequence
  Made in a lab
QDIQMTQSPLSLSASVGDRVTITCQASRDISIYLNWYQQKPGKVPKLLIFGASTLRSGVPSRFSGSGSGTDFTLTITSLQPEDVATYYCQKYNSAPYTFGPGTKVEIR

SEQ ID NO: 391 - protein, 108 aa, Artificial Sequence
  Made in a lab
QDIQMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKVEIK

SEQ ID NO: 392 - protein, 108 aa, Artificial Sequence
  Made in a lab
QDIQMTQSPATLSLSPGERATLSCRASQSINTYLAWYQQKPGHPPRLLIYDASNRATGIPDRFSGSGSGTDFTLTISRLEPEDFVVYYCQQYGRSRYTFGQGTKLEIK

SEQ ID NO: 393 - protein, 108 aa, Artificial Sequence
  Made in a lab
QDIQMTQSPSSLSASVGDRVTITCRASQGISNYLAWYQQKPGKVPKLLIYAASTLQSGVPSRFSGSGSGTEFTLTISSLQPEDFATYYCQQSDNIPYTFGLGTKLEIK

SEQ ID NO: 394 - protein, 110 aa, Artificial Sequence
  Made in a lab
QSELTQPPSASGTPGQRVTISCSGSSSNIGSNTVNWYQQLPGTAPKLLIYSNNQRPSGVPDRFSGSKSGTSASLAISGLQSEDEADYYCAAWDDSLNGYVFGIGTKVTVL

SEQ ID NO: 395 - protein, 108 aa, Artificial Sequence
  Made in a lab
QDIQMTQSPSSLSASVGDSVTIACRASQSISTYLNWYQQKPGQAPKLLIYEATNLQSGVPSRFSGSGSGTDFTLTISSLQPEDVATYYCQQANNFPFTFGPGTKVDIK

SEQ ID NO: 396 - protein, 110 aa, Artificial Sequence
  Made in a lab
QSVLTQPPSASGTPGQRVTISCSGSSSNIGGNAVNWYQQLPGTAPKLVLYSSNHRPSGVPDRFSGSRSGTSASLAISGLQSQDEADYYCAAWDDSLNGPLFGGGTKLTVL

SEQ ID NO: 397 - protein, 108 aa, Artificial Sequence
  Made in a lab
QSELTQPPSVSVSPGQTATISCSGEKLGDRYAWWYQQKAGQSPVLVIYQDTKRPSGVPDRFSGSNSGTSASLVISGLQSEDEADYYCAAWDDSLKGVVFGGGSKLTVL

SEQ ID NO: 398 - protein, 110 aa, Artificial Sequence
  Made in a lab
QSELTQPPSASGTPGQRVTISCSGRSSNIGSNSVNWYQQLPGTAPKLLIYSNNHRPSGVPDRFSGSKSGTSASLAISGLQSEDEADYYCAAWDDSLYQGVFGGGTKLTVL

SEQ ID NO: 399 - protein, 108 aa, Artificial Sequence
  Made in a lab
QDIQMTQSPSSLSASVGDRVTITCRASQSISSYLNWYQQKPGKAPKLLIYAASSLQSGVPSKFSGSGSGTDFTLTISSLLPEDFATYYCQQYEYFPPTFGQGTKVEIK

SEQ ID NO: 400 - protein, 109 aa, Artificial Sequence
  Made in a lab
QDIQMTQSPDSLSLSPGERATLSCRASQSVSSSYLAWYQQKPGQAPRLLIYGASSRATGIPDRFSGSGSGTDFTLTISRLEPEDFAVYYCQQYGSSSLTFGGGTEVEIK

SEQ ID NO: 401 - protein, 108 aa, Artificial Sequence
  Made in a lab
QDIQMTQSPATLSASVGDRVTITCRSSQRISGYLNWYQQQPGKAPKLLIYAASTLQSGVPSRISGSGSGTDFSLIISSLQPEDVATYYCQQSYSPPFTFGQGTKVEIK

SEQ ID NO: 402 - protein, 108 aa, Artificial Sequence
  Made in a lab
QDIQMTQSPSTLSASVGDRVTITCRASQSISTWLAWFQQKPGKAPKLLIYKASSLQSGVPSRFSGSGSGTDFTLTISSLQPEDFATYYCQQYKSYPFTFGGGTKVEIK

SEQ ID NO: 403 - protein, 108 aa, Artificial Sequence
  Made in a lab
QDIQMTQSPSSLSASVGDSVTITCQASQDIRSYLNWFQQKPGKAPKLLIYDTVNLQTGVPSRFSGSGSGTDYSFTINNLQPEDFATYYCQQYEYLPITFGQGTRLEIK

SEQ ID NO: 404 - protein, 108 aa, Artificial Sequence
  Made in a lab
QDIQMTQSPSSLSASVGDRVTITCRASQSISNYLNWYQQKSGKAPNLLMYAASSLHSGVSSRFSGSGSGTEFTLTISSLQPEDYATYYCQQSYSTSGTFGQGTKVEIK

SEQ ID NO: 405 - protein, 108 aa, Artificial Sequence
  Made in a lab
QDIQMTQSPSSLSASVGDRVTITCRASQSISSYLNWYQQKPGKAPKLLIYAASSLQSGVPSRFSGSGSGTDFTLTISSLQPEDFATYYCQQSYSTPVTFGGGTKVEIK

SEQ ID NO: 406 - protein, 109 aa, Artificial Sequence
  Made in a lab
QDIQMTQSPSSLSASVGDRVAITCRASQSIDTYLNWYQHKPGKAPKLLIYAASSLQSGVPSRFSGSGSGTDFTLTISGLQPEDFASYFCQQSYSSPGITFGGGTRVEIK

SEQ ID NO: 407 - protein, 108 aa, Artificial Sequence
  Made in a lab
QDIQMTQSPSSLSASVGDRVTITCRASQSIGTYLNWYQQKPGKAPNLLIYGTSSLQRGVPSRFSGSGSGTDFTLTISTLQPEDFVHYYCQQSYTSPPTFGQGTKVEVK

SEQ ID NO: 408 - protein, 106 aa, Artificial Sequence
  Made in a lab
QYELTQSPSVSVSPGQTASIPCSGDQLGGKFTSWYQQKAGQSPLLVIYQDTKRPSGIPERFSGSNSGNTATLTITGTQAMDEADYYCLVWDSNTYVFGPGTKVTVL

SEQ ID NO: 409 - protein, 108 aa, Artificial Sequence
  Made in a lab
QYELTQPPSVSVAPGQTARITCGGNNIGSKSVHWYQQKPGQAPVLVVYDDSDRPSGIPERFSGSNSGNTATLTVSRVEAGDEADYYCQVWDSSGGLQVFGTGTKVTVL

SEQ ID NO: 410 - protein, 108 aa, Artificial Sequence
  Made in a lab
QDIQMTQSPSTLSASVGDRVTITCRASQSISSWLAWYQQKPGKAPKLLIYKASNLESEVPSRFSGSGSGTEFTLTISSLQPDDFATYYCQQYNAYPFIFGPGTKVDIK

SEQ ID NO: 411 - protein, 107 aa, Artificial Sequence
  Made in a lab
QDIQMTQSPSTLSASVGDRVTITCRASQSISSWLAWYQQKPGKAPKLLIYKASSLESGVPSRFSGSGSGTEFTLTISSLQPDDFATYHCQQYKSYWTFGQGTKVEIK

SEQ ID NO: 412 - protein, 110 aa, Artificial Sequence
  Made in a lab
QSELTQPPSVSEAPRQRVTVSCSGSPSNIGSNSVNWYQQLPGKAPKVVIYYDDLVPSGVSDRFSGSKSGTSASLAISGLQSEDEADYYCAAWDDRLNGWVFGGGTKLTVL

SEQ ID NO: 413 - protein, 108 aa, Artificial Sequence; identical to SEQ ID NO: 391
  Made in a lab

SEQ ID NO: 414 - protein, 108 aa, Artificial Sequence
  Made in a lab
QDIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIK

SEQ ID NO: 415 - protein, 108 aa, Artificial Sequence
  Made in a lab
QDIQMTQSPGTLSLSPGERATLSCRASQSVSSSYLAWYQQKPGQAPRLLIYGASNRVTGIPDRFSGSGSGTDFTLTISRLEPEDSAVYYCHQYGSQYTFGPGTKLEIK

SEQ ID NO: 416 - protein, 108 aa, Artificial Sequence
  Made in a lab
QDIQMTQSPSSLSASVGDRVTISCQASQHISNFLNWYQQKPGKAPKLLIYDTSNLRSGVPSRFSGSGSGTDFTFTISSLQPEDIATYYCQQYDNFPLTFGGGTKVEIK

SEQ ID NO: 417 - protein, 110 aa, Artificial Sequence
  Made in a lab
QSELTQPPSVSEAPGQRVTISCSGSTSNIGNNAVIWYQQLPGKAPKLLIYYDDLLPSGVSDRFSGSKSGTSGSLAISGLQSEDEADYYCASWDDNLNGPLFGGGTKLTVL

SEQ ID NO: 418 - protein, 108 aa, Artificial Sequence
  Made in a lab
QDIQMTQSPSTLSASVGDRVTITCRASQSISSWLAWYQQKPGKAPKLLIYQASTLESGVPSRFSGSGSGTDFTLTISSLQPEDFATYYCQQANSFPITFGGGTKVEIN

SEQ ID NO: 419 - protein, 108 aa, Artificial Sequence
  Made in a lab
QDIQMTQSPSSLSASVGDRVTITCQASQDITNYLNWYLHKPGKAPKLLIYDASNLQTGVPSRFSGSGSGTDFTFTITSLQPEDFGTYYCQQYDTLHPSFGPGTTVDIK

SEQ ID NO: 420 - protein, 110 aa, Artificial Sequence
  Made in a lab
QSALTQPPSASGSPGQSVTISCTGTSSDVGVYDSVSWYQQHPGKAPKLMIYDVSDRPSGVSNRFSGSKSGYTASLTISALQAEDEADYYCGSYRASSSYVFGTGTKVTVL

SEQ ID NO: 421 - protein, 108 aa, Artificial Sequence
  Made in a lab
QDIQMTQSPSSLSASVGDRVTITCRASQNIYHYLHWYQQKPGKAPKLLIYAASTLESGVPSRFSGSGSGTDFTLTINSLQPEDFATYYCQQTYDTPLTFGGGSKVEVK

SEQ ID NO: 422 - protein, 107 aa, Artificial Sequence
  Made in a lab
QSALTQPPSVSVSPGQTASITCSGDKLGEKFASWYQQRPGQSPILVIYQDSKRPSGIPERFSGSNSGNTATLTISGTQTMDEADYYCQAWGGSTAYVFGSGTKVTVL

SEQ ID NO: 423 - protein, 110 aa, Artificial Sequence
  Made in a lab
QDIQMTQSPATLSLSPGERATLSCRASQSVTTYLAWYQQRPGQAPRLLIYDASNRATGIPARFSGSGSGTDFTLTISSVEPEDYAVYYCQQRSNWPPSITFGQGTRLESK

SEQ ID NO: 424 - protein, 110 aa, Artificial Sequence
  Made in a lab
QSALTQPASVSGSPGQSITISCTGTSSDVGSYNLVSWYQQHPGKAPKVMIYEGSKRPSGVSNRFSGSKSGNTASLTISGLQAEDEADYYCCSYAGRSTYVFGTGTKVTVL

SEQ ID NO: 425 - protein, 107 aa, Artificial Sequence
  Made in a lab
QSELTQTPSVSVSPGQTASITCSGNRLEDKYVHWYQQKPGQSPVLVLYQDNKRPSGIPERFSGSNSGNTATLTISETQTLDEADYYCQAWDSSTREVFGGGTKLTVL

SEQ ID NO: 426 - protein, 108 aa, Artificial Sequence
  Made in a lab
QDIQMTQSPSSVSASVGDTVTITCRASQGVSDWLAWYQQKPGKAPNLLIYGASSLQTGVPSRFSGGGSGTDFTLTISSLQPEDFATYYCQQAHSFPFTFGGGTRVEIK

SEQ ID NO: 427 - protein, 109 aa, Artificial Sequence
  Made in a lab
QDIQMTQSPATLSVSPGERATLSCRASQNINTHLAWYQQKPGQAPRLLIFGASTRATGIPARFSGSGSGTEFTLTISSLQSEDFTVYYCQQYGSSLTWTFGQGTKVEIK

SEQ ID NO: 428 - protein, 109 aa, Artificial Sequence
  Made in a lab
QDIQMTQSPGTLSLSPGERATLSCRASQSVSGNYLAWYQQKLGQAPRLLIYGASSRATGTPDRFSGSGSGTEFTLTISSLQPEDFATYYCLQHNSYPLTSGSGTEFTLT

SEQ ID NO: 429 - protein, 108 aa, Artificial Sequence
  Made in a lab
QSELTQPPSVSVAPGETATVTCGGINIGSKSVHWYQQKPGQAPVVVMYFDSDRPSGIPERFSGSNSGDTATLTISRVEAGDEADYYCQVWDSRSDQYVFGFGTKVTVL

SEQ ID NO: 430 - protein, 108 aa, Artificial Sequence
  Made in a lab
QDIQMTQSPSTLSASVGDRVTITCRASQSISSWLAWYQQKPGKAPKLLIYKASTLESGVPSRFSAGGSGTEFTLTISSLQPDDFGTYYCQQYNSYSLTFGGGTKVEII

SEQ ID NO: 431 - protein, 108 aa, Artificial Sequence
  Made in a lab
QDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 432 - protein, 108 aa, Artificial Sequence
  Made in a lab
QDIQMTQSPSSLSASVGDRVTITCRASQSINNYLNWYQQKPGKAPKLLIYTASTLHSGVPSRFSGSGSGTDFTLTISNLQPEDFATYYCQQSYATLRTFGQGTKVEIK

SEQ ID NO: 433 - protein, 110 aa, Artificial Sequence
  Made in a lab
QSVLTQPASVSGSPGQSITISCSGSSSDVGHYDYVSWYQQHPGKAPKLMIYDVSNRPSGVSNRFSGSKSGNTASLTISGLQADDEAEYYCSSYTSSGTRVFGTGTKVTVL

SEQ ID NO: 434 - protein, 108 aa, Artificial Sequence
  Made in a lab
QDIQMTQSPSTLSASVGDRVTFTCRASQSVNNWVAWYQQKPGKAPKLLIYAASSLQSGVPSRFSGSGSGTDFTLTISSLQPEDFATYYCQQSYSMPLTFGGGTKVEIK

SEQ ID NO: 435 - protein, 118 aa, Artificial Sequence
  Made in a lab
EVQLLESGGGLVQPGGSLRLSCAASGFTFSKYSMHWVRQAPGKGLEWVSSIWPSGGWTTYADSVKGRFTISRDNSKNTLYLQMNSLRAEDTAVYYCARGGYYYALDYWGQGNLVTVSS

SEQ ID NO: 436 - protein, 122 aa, Artificial Sequence
  Made in a lab
EVQLLESGGGLVQPGGSLRLSCAASGFTFSFYQMHWVRQAPGKGLEWVSGIYPSGGYTKYADSVKGRFTISRDNSKNTLYLQMNSLRAEDTAVYYCARDLGYGSSWYYFDYWGQGTLVTVSS

SEQ ID NO: 437 - protein, 117 aa, Artificial Sequence
  Made in a lab
EVQLLESGGGLVQPGGSLRLSCAASGFTFSKYSMHWVRQAPGKGLEWVSGISPSGYTRYADSVKGRFTISRDNSKNTLYLQMNSLRAEDTAVYYCARDQLLEAFDIWGQGTTVTVSS

SEQ ID NO: 438 - protein, 119 aa, Artificial Sequence
  Made in a lab
EVQLLESGGGLVQPGGSLRLSCAASGFTFSEYPMNWVRQAPGKGLEWVSGIYSSGGLTKYADSVKGRFTISRDNSKNTLYLQMNSLRAEDTAVYYCARDHYGDYAFDYWGQGTLVTVSS

SEQ ID NO: 439 - protein, 126 aa, Artificial Sequence
  Made in a lab
EVQLLESGGGLVQPGGSLRLSCAASGFTFSLYEMVWVRQAPGKGLEWVSGIWPSGGWTQYADSVKGRFTISRDNSKNTLYLQMNSLRAEDTAVYYCARDLAVAGWDYYYYYGMDVWGQGTTVTVSS

SEQ ID NO: 440 - protein, 125 aa, Artificial Sequence
  Made in a lab
EVQLLESGGGLVQPGGSLRLSCAASGFTFSQYNMIWVRQAPGKGLEWVSWISSSGGTEYADSVKGRFTISRDNSKNTLYLQMNSLRAEDTAVYYCAKDRGYGSGSYGAYDAFDIWGQGKMVTVSS

SEQ ID NO: 441 - protein, 118 aa, Artificial Sequence
  Made in a lab
EVQLLESGGGLVQPGGSLRLSCAASGFTFSHYSMGWVRQAPGKGLEWVSSIYPSGGSTLYADSVKGRFTISRDNSKNTLYLQMNSLRAEDTAVYYCARWGVGATFDYWGQGTLVTVSS

SEQ ID NO: 442 - protein, 118 aa, Artificial Sequence
  Made in a lab
EVQLLESGGGLVQPGGSLRLSCAASGFTFSHYMMAWVRQAPGKGLEWVSYIYPSGGWTSYADSVKGRFTISRDNSKNTLYLQMNSLRAEDTAVYYCARFDYTIGFDFWGQGTLVTVSS

SEQ ID NO: 443 - protein, 120 aa, Artificial Sequence
  Made in a lab
EVQLLESGGGLVQPGGSLRLSCAASGFTFSHYGMTWVRQAPGKGLEWVSGIRSSGGVTNYADSVKGRFTISRDNSKNTLYLQMNSLRAEDTAVYYCAREGSGWSKAFDIWGQGTMVTVSS

SEQ ID NO: 444 - protein, 127 aa, Artificial Sequence
  Made in a lab
EVQLLESGGGLVQPGGSLRLSCAASGFTFSHYNMRWVRQAPGKGLEWVSSISPSGGFTGYADSVKGRFTISRDNSKNTLYLQMNSLRAEDTAVYYCARGINYYDSSGYYPPVGMDVWGQGTTVTVSS

SEQ ID NO: 445 - protein, 123 aa, Artificial Sequence
  Made in a lab
EVQLLESGGGLVQPGGSLRLSCAASGFTFSWYNMLWVRQAPGKGLEWVSSISPSGGYTVYADSVKGRFTISRDNSKNTLYLQMNSLRAEDTAVYYCARDRGGSPFRPDAFDIWGQGTMVTVSS

SEQ ID NO: 446 - protein, 125 aa, Artificial Sequence
  Made in a lab
EVQLLESGGGLVQPGGSLRLSCAASGFTFSEYDMLWVRQAPGKGLEWVSSIWPSGGFTQYADSVKGRFTISRDNSKNTLYLQMNSLRAEDTAVYYCARNYYDFWSGPYYYGMDVWGQGTTVTVSS

SEQ ID NO: 447 - protein, 122 aa, Artificial Sequence
  Made in a lab
EVQLLESGGGLVQPGGSLRLSCAASGFTFSGYVMGWVRQAPGKGLEWVSSISPSGGYTLYADSVKGRFTISRDNSKNTLYLQMNSLRAEDTAVYYCARAFSTRWYWGAFDIWGQGTMVTVSS

SEQ ID NO: 448 - protein, 125 aa, Artificial Sequence
  Made in a lab
EVQLLESGGGLVQPGGSLRLSCAASGFTFSIYHMNWVRQAPGKGLEWVSGISSSGGRTNYADSVKGRFTISRDNSKNTLYLQMNSLRAEDTAVYYCASSYRAAGWVDYYYGMDVWGQGTTVTVSS

SEQ ID NO: 449 - protein, 121 aa, Artificial Sequence
  Made in a lab
EVQLLESGGGLVQPGGSLRLSCAASGFTFSLYDMHWVRQAPGKGLEWVSRIYSSGGTTQYADSVKGRFTISRDNSKNTLYLQMNSLRAEDTAMYYCARDPGYGSHHSFDYWGQGTLVTVSS

SEQ ID NO: 450 - protein, 117 aa, Artificial Sequence
  Made in a lab
EVQLLESGGGLVQPGGSLRLSCAASGFTFSSYYMTWVRQAPGKGLEWVSGIYSSGGPTQYADSVKGRFTISRDNSKNTLYLQMNSLRAEDTAVYYCARAGGDAFDIWGQGTMVTVSS

SEQ ID NO: 451 - protein, 118 aa, Artificial Sequence
  Made in a lab
EVQLLESGGGLVQPGGSLRLSCAASGFTFSSYEMFWVRQAPGKGLEWVSSISPSGGYTYYADSVKGRFTISRDNSKNTLYLQMNSLRAEDTAVYYCATMTTSGFHLIWGQGTMVTVSS

SEQ ID NO: 452 - protein, 117 aa, Artificial Sequence
  Made in a lab
EVQLLESGGGLVQPGGSLRLSCAASGFTFSDYPMQWVRQAPGKGLEWVSWIGPSGGWTVYADSVKGRFTISRDNSKNTLYLQMNSLRAEDTAVYYCARDDGIAGFLWGQGTLVTVSS

SEQ ID NO: 453 - protein, 125 aa, Artificial Sequence
  Made in a lab
EVQLLESGGGLVQPGGSLRLSCAASGFTFSWYLMHWVRQAPGKGLEWVSGIWPSGGHTLYADSVKGRFTISRDNSKNTLYLQMNSLRAEDTAVYYCAREPLLWFGELSYNWFDPWGQGTLVTVSS

SEQ ID NO: 454 - protein, 123 aa, Artificial Sequence
  Made in a lab
EVQLLESGGGLVQPGGSLRLSCAASGFTFSYYENMAWVRQAPGKGLEWVSGIYPSGGLTYYADSVKGRFTISRDNSKNTLYLQMNSLRAEDTAVYYCARSRRYYDSSDAFDIWGQGTMVTVSS

SEQ ID NO: 455 - protein, 123 aa, Artificial Sequence
  Made in a lab
EVQLLESGGGLVQPGGSLRLSCAASGFTFSTYVMYWVRQAPGKGLEWVSGIGPSGGNTFYADSVKGRFTISRDNSKNTLYLQMNSLRAEDTAVYYCARDPGDFWSGYYGMDVWGQGTTVTVSS

SEQ ID NO: 456 - protein, 122 aa, Artificial Sequence
  Made in a lab
EVQLLESGGGLVQPGGSLRLSCAASGFTFSFYGMAWVRQAPGKGLEWVSSIYPSGGYTDYADSVKGRFTISRDNSKNTLYLQMNSLRAEDTAVYYCARSAGGWIGGGAFDIWGQGTMVTVSS

SEQ ID NO: 457 - protein, 126 aa, Artificial Sequence
  Made in a lab
EVQLLESGGGLVQPGGSLRLSCAASGFTFSKYPMMWVRQAPGKGLEWVSYIYPSGGKTAYADSVKGRFTISRDNSKNTLYLQMNSLRAEDTAVYYCATYGSGSYYLYYYYYYMDVWGKGTTVTVSS

SEQ ID NO: 458 - protein, 125 aa, Artificial Sequence
  Made in a lab
EVQLLESGGGLVQPGGSLRLSCAASGFTFSWYSMWWVRQAPGKGLEWVSYIVPSGGETIYADSVKGRFTISRDNSKNTLYLQMNSLRAEDTAVYYCAKNLGEGFWSDYYPPLDYWGQGTLVTVSS

SEQ ID NO: 459 - protein, 118 aa, Artificial Sequence
  Made in a lab
EVQLLESGGGLVQPGGSLRLSCAASGFTFSHYGMHWVRQAPGKGLEWVSSIYPGMTQYADSVKGRFTISRDNSKNTLYLQMNSLRAEDTAVYYCARDRGSGIDAFDIWGQGTMVTVSS

SEQ ID NO: 460 - protein, 125 aa, Artificial Sequence
  Made in a lab
EVQLLESGGGLVQPGGSLRLSCAASGFTFSWYDMLWVRQAPGKGLEWVSVISPSGGRTFYADSVKGRFTISRDNSKNTLYLQMNSLRAEDTAVYYCAVTRSMYSSSWYGAPPTHWGQGTLVTVSS

SEQ ID NO: 461 - protein, 120 aa, Artificial Sequence
  Made in a lab
EVQLLESGGGLVQPGGSLRLSCAASGFTFSWYKMHWVRQAPGKGLEWVSGISSSGGLTKYADSVKGRFTISRDNSKNTLYLQMNSLRAEDTAVYYCTTERRGDGGAFDIWGQGTMVTVSS

SEQ ID NO: 462 - protein, 122 aa, Artificial Sequence
  Made in a lab
EVQLLESGGGLVQPGGSLRLSCAASGFTFSEYTMYWVRQAPGKGLEWVSYISPSGGTTNYADSVKGRFTISRDNSKNTLYLQMNSLRAEDTAVYYCAKGAWGDIYYYGMDVWGQGTTVTVSS

SEQ ID NO: 463 - protein, 115 aa, Artificial Sequence
  Made in a lab
EVQLLESGGGLVQPGGSLRLSCAASGFTFSSYWMHWVRQAPGKGLEWVSSIYPSGGSTEYADSVKGRFTISRDNSKNTLYLQMNSLRAEDTAVYYCAKGIWFDPWGQGTLVTVSS

SEQ ID NO: 464 - protein, 121 aa, Artificial Sequence
  Made in a lab
EVQLLESGGGLVQPGGSLRLSCAASGFTFSRYGMMWVRQAPGKGLEWVSYISSSGGFTRYADSVKGRFTISRDNSKNTLYLQMNSLRAEDTAVYYCARVGGYSYGPHFDFWGQGTLVTVSS

SEQ ID NO: 465 - protein, 123 aa, Artificial Sequence
  Made in a lab
EVQLLESGGGLVQPGGSLRLSCAASGFTFSWYHMIWVRQAPGKGLEWVSWISPSGGFTKYADSVKGRFTISRDNSKNTLYLQMNSLRAEDTAVYYCARYDSRAAAGTNAFDIWGQGTMVTVSS

SEQ ID NO: 466 - protein, 123 aa, Artificial Sequence
  Made in a lab
EVQLLESGGGLVQPGGSLRLSCAASGFTFSPYKMVWVRQAPGKGLEWVSGISPSGGYTYYADSVKGRFTISRDNSKNTLYLQMNSLRAEDTAVYYCARGGYGWSYYYYGMDVWGQGTTVTVSS

SEQ ID NO: 467 - protein, 118 aa, Artificial Sequence
  Made in a lab
EVQLLESGGGLVQPGGSLRLSCAASGFTFSNYWMYWVRQAPGKGLEWVSVISSSGGHTFYADSVKGRFTISRDNSKNTLYLQMNSLRAEDTAVYYCARDYEGGSNDYWGQGTLVTVSS

SEQ ID NO: 468 - protein, 120 aa, Artificial Sequence
  Made in a lab
EVQLLESGGGLVQPGGSLRLSCAASGFTFSNYAMSWVRQAPGKGLEWVSSIYSSGGYTAYADSVKGRFTISRDNSKNTLYLQMNSLRAEDTAMYYCARVPHVFRGELDYWGQGTLVTVSS

SEQ ID NO: 469 - protein, 122 aa, Artificial Sequence
  Made in a lab
EVQLLESGGGLVQPGGSLRLSCAASGFTFSFYTMWWVRQAPGKGLEWVSSIYPSGGQTLYADSVKGRFTISRDNSKNTLYLQMNSLRAEDTAVYYCARPDSYGYLYYGMDVWGQGTTVTVSS

SEQ ID NO: 470 - protein, 115 aa, Artificial Sequence
  Made in a lab
EVQLLESGGGLVQPGGSLRLSCAASGFTFSWYPMEWVRQAPGKGLEWVSGIGPSGGQTTYADSVKGRFTISRDNSKNTLYLQMNSLRAEDTAVYYCARGSYSFIWGQGTMVTVSS

SEQ ID NO: 471 - protein, 125 aa, Artificial Sequence
  Made in a lab
EVQLLESGGGLVQPGGSLRLSCAASGFTFSLYKMAWVRQAPGKGLEWVSVIGSSGGRTPYADSVKGRFTISRDNSKNTLYLQMNSLRAEDTAVYYCARAPLSGWFGQAHDAFDIWGQGTMVTVSS

SEQ ID NO: 472 - protein, 122 aa, Artificial Sequence
  Made in a lab
EVQLLESGGGLVQPGGSLRLSCAASGFTFSTYEMNWVRQAPGKGLEWVSGIVPSGGVTYYADSVKGRFTISRDNSKNTLYLQMNSLRAEDTAVYYCARGPYSYGHDYGMDVWGQGTTVTVSS

SEQ ID NO: 473 - protein, 112 aa, Artificial Sequence
  Made in a lab
EVQLLESGGGLVQPGGSLRLSCAASGFTFSVYPMHWVRQAPGKGLEWVSSIYSSGGFTMYADSVKGRFTISRDNSKNTLYLQMNSLRAEDTAEGVADAFDIWGQGTMVTVSS

SEQ ID NO: 474 - protein, 120 aa, Artificial Sequence
  Made in a lab
EVQLLESGGGLVQPGGSLRLSCAASGFTFSSYTMHWVRQAPGKGLEWVSSISPSGGMTFYADSVKGRFTISRDNSKNTLYLQMNSLRAEDTAVYYCARTYDFWSGYFDYWGQGTLVTVSS

SEQ ID NO: 475 - protein, 125 aa, Artificial Sequence
  Made in a lab
EVQLLESGGGLVQPGGSLRLSCAASGFTFSTYEMGWVRQAPGKGLEWVSVIWSSGGHTWYADSVKGRFTISRDNSKNTLYLQMNSLRAEDTAVYYCARSNQGDFWSGYPFAFDIWGQGTMVTVSS

SEQ ID NO: 476 - protein, 120 aa, Artificial Sequence
  Made in a lab
EVQLLESGGGLVQPGGSLRLSCAASGFTFSNYNMYWVRQAPGKGLEWVSYISPSGGMTKYADSVKGRFTISRDNSKNTLYLQMNSLRAEDTAVYYCARRGVLGYYGMDVWGQGTTVTVSS

SEQ ID NO: 477 - protein, 123 aa, Artificial Sequence
  Made in a lab
EVQLLESGGGLVQPGGSLRLSCAASGFTFSLYSMNWVRQAPGKGLEWVSSISSSGGATLYADSVKGRFTISRDNSKNTLYLQMNSLRAEDTAVYYCARDLISSGYYPDAFDIWGQGTMVTVSS

SEQ ID NO: 478 - protein, 128 aa, Artificial Sequence
  Made in a lab
EVQLLESGGGLVQPGGSLRLSCAASGFTFSQYMMHWVRQAPGKGLEWVSSISSSGGWTSYADSVKGSFTISRDNSKNTLYLQMNSLRAEDTAVYYCARPLALGYDFWSGYQAAGFDYWGQGTLVTVSS

SEQ ID NO: 479 - protein, 119 aa, Artificial Sequence
  Made in a lab
EVQLLESGGGLVQPGGSLRLSCAASGFTFSWYSMVWVRQAPGKGLEWVSSIVPSGGLTWYADSVKGRFTISRDNSKNTLYLQMNSLRAEDTAVYYCARDVYGDYYFDNWGQGTLVTVSS

SEQ ID NO: 480 - protein, 119 aa, Artificial Sequence
  Made in a lab
EVQLLESGGGLVQPGGSLRLSCAASGFTFSYYNMTWVRQAPGKGLEWVSSISPSGGRTGYADSVKGRFTISRDNSKNTLYLQMNSLRAEDTAVYYCARVSGFYAAFDYWGQGTLVTVSS

SEQ ID NO: 481 - protein, 120 aa, Artificial Sequence
  Made in a lab
EVQLLESGGGLVQPGGSLRLSCAASGFTFSWYWMTWVRQAPGKGLEWVSSISPSGGHTSYADSVKGRFTISRDNSKNTLYLQMNSLRAEDTAVYYCAKSSGWYEDYFDYWGQGTLVTVSS

SEQ ID NO: 482 - protein, 123 aa, Artificial Sequence
  Made in a lab
EVQLLESGGGLVQPGGSLRLSCAASGFTFSPYEMAWVRQAPGKGLEWVSVIGPSGGYTEYADSVKGRFTISRDNSKNTLYLQMNSLRAEDTAVYYCARGYDFWSGYYDAFDIWGQGTMVTVSS

SEQ ID NO: 483 - protein, 119 aa, Artificial Sequence
  Made in a lab
EVQLLESGGGLVQPGGSLRLSCAASGFTFSSYFMGWVRQAPGKGLEWVSSIWPSGGNTTYADSVKGRFTISRDNSKNTLYLQMNSLRAEDTAVYYCARHVGWGRYFDYWGQGTLVTVSS

SEQ ID NO: 484 - protein, 124 aa, Artificial Sequence
  Made in a lab
EVQLLESGGGLVQPGGSLRLSCAASGFTFSAYRMIWVRQAPGKGLEWVSYISSSGGRTDYADSVKGRFTISRDNSKNTLYLQMNSLRAEDTAVYYCTTGGLRYFDWLAPSMDYWGQGTLVTVSS

SEQ ID NO: 485 - protein, 116 aa, Artificial Sequence
  Made in a lab
EVQLLESGGGLVQPGGSLRLSCAASGFTFSAYSMVWVRQAPGKGLEWVSYIYPSGGITTYADSVKGRFTISRDNSKNTLYLQMNSLRAEDTAVYYCAREGQVFDIWGQGTTVTVSS

SEQ ID NO: 486 - protein, 118 aa, Artificial Sequence
  Made in a lab
EVQLLESGGGLVQPGGSLRLSCAASGFTFSNYAMGWVRQAPGKGLEWVSRIVPSGGMTSYADSVKGRFTISRDNSKNTLYLQMNSLRAEDTAVYYCTTDDFWSGMDVWGQGTTVTVSS

SEQ ID NO: 487 - protein, 121 aa, Artificial Sequence
  Made in a lab
EVQLLESGGGLVQPGGSLRLSCAASGFTFSLYNMQWVRQAPGKGLEWVSGISLSGGKTFYADSVKGRFTISRDNSKNTLYLQMNSLRAEDTAVYYCARDYGVATLDAFDIWGQGTMVTVSS

SEQ ID NO: 488 - protein, 127 aa, Artificial Sequence
  Made in a lab
EVQLLESGGGLVQPGGSLRLSCAASGFTFSAYTMHWVRQAPGKGLEWVSSIYPSGGTTPYADSVKGRFTISRDNSKNTLYLQMNSLRAEDTATYYCARVVNIDFWSGYNMRSAFDIWGQGTMVTVSS

SEQ ID NO: 489 - protein, 122 aa, Artificial Sequence
  Made in a lab
EVQLLESGGGLVQPGGSLRLSCAASGFTFSDYLMGWVRQAPGKGLEWVSVISSSGGPTAYADSVKGRFTISRDNSKNTLYLQMNSLRAEDTAVYYCARVGLDYGILGAFDIWGQGTMVTVSS

SEQ ID NO: 490 - protein, 120 aa, Artificial Sequence
  Made in a lab
EVQLLESGGGLVQPGGSLRLSCAASGFTFSEYGMSWVRQAPGKGLEWVSSIRSSGGWTKYADSVKGRFTISRDNSKNTLYLQMNSLRAEDTAVYYCAKGLGATSGEFDYWGQGTLVTVSS

SEQ ID NO: 491 - protein, 121 aa, Artificial Sequence
  Made in a lab
EVQLLESGGGLVQPGGSLRLSCAASGFTFSKYDMWWVRQAPGKGLEWVSSIWPSGGWTSYADSVKGRFTISRDNSKNTLYLQMNSLRAEDTAVYYCAKSRGSPWYGDFDHWGQGTLVTVSS

SEQ ID NO: 492 - protein, 118 aa, Artificial Sequence
  Made in a lab
EVQLLESGGGLVQPGGSLRLSCAASGFTFSMYTMHWVRQAPGKGLEWVSWISPSGGWTKYADSVKGRFTISRDNSKNTLYLQMNSLRAEDTAVYYCAVSPWGGPFDYWGQGTLVTVSS

SEQ ID NO: 493 - protein, 123 aa, Artificial Sequence
  Made in a lab
EVQLLESGGGLVQPGGSLRLSCAASGFTFSQYFMMWVRQAPGKGLEWVSSISPSGGYTQYADSVKGRFTISRDNSKNTLYLQMNSLRAEDTAVYYCAKTGDYTRYYSYGMDVWGQGTLVTVSS

SEQ ID NO: 494 - protein, 124 aa, Artificial Sequence
  Made in a lab
EVQLLESGGGLVQPGGSLRLSCAASGFTFSGYSMAWVRQAPGKGLEWVSGIYSSGGWTWYADSVKGRFTISRDNSKNTLYLQMNSLRAEDTAVYYCTRDQSFSYDSSLDAFDIWGQGTMVTVSS

SEQ ID NO: 495 - protein, 124 aa, Artificial Sequence
  Made in a lab
EVQLLESGGGLVQPGGSLRLSCAASGFTFSLYGMHWVRQAPGKGLEWVSSIVPSGGLTRYADSVKGRFTISRDNSKNTLYLQMNSLRAEDTATYYCARLAYYDFWSGRDAFDIWGQGTMVTVSS

SEQ ID NO: 496 - protein, 118 aa, Artificial Sequence
  Made in a lab
EVQLLESGGGLVQPGGSLRLSCAASGFTFSHYGMDWVRQAPGKGLEWVSSIYSSGGYTLYADSVKGRFTISRDNSKNTLYLQMNSLRAEDTATYYCATRTWADAFDVWGQGTTVTVSS

SEQ ID NO: 497 - protein, 2 aa, Artificial Sequence
  Made in a lab
NS

SEQ ID NO: 498 - protein, 6 aa, Artificial Sequence
  Made in a lab
SCPPCP

SEQ ID NO: 499 - protein, 8 aa, Artificial Sequence
  Made in a lab
GGGGSGNS

SEQ ID NO: 500 - protein, 8 aa, Artificial Sequence
  Made in a lab
GCPPCPNS

SEQ ID NO: 501 - protein, 8 aa, Artificial Sequence
  Made in a lab
GSPPSPNS

SEQ ID NO: 502 - protein, 8 aa, Artificial Sequence
  Made in a lab
GSPPSPNS

SEQ ID NO: 503 - protein, 8 aa, Artificial Sequence
  Made in a lab
GCPPCPNS

SEQ ID NO: 504 - protein, 8 aa, Artificial Sequence
  Made in a lab
GCPPCPNS

SEQ ID NO: 505 - protein, 9 aa, Artificial Sequence
  Made in a lab
GCPPCPGNS

SEQ ID NO: 506 - protein, 9 aa, Artificial Sequence
  Made in a lab
GCPPCPANS

SEQ ID NO: 507 - protein, 9 aa, Artificial Sequence
  Made in a lab
GCPPCPANS

SEQ ID NO: 508 - protein, 9 aa, Artificial Sequence
  Made in a lab
EEEEDEGNS

SEQ ID NO: 509 - protein, 10 aa, Artificial Sequence
  Made in a lab
NYGGGGSGNS

SEQ ID NO: 510 - protein, 10 aa, Artificial Sequence
  Made in a lab
VSERPFPPNS

SEQ ID NO: 511 - protein, 11 aa, Artificial Sequence
  Made in a lab
EPKSCDKTCCP

SEQ ID NO: 512 - protein, 11 aa, Artificial Sequence
  Made in a lab
SQPEIVPISNS

SEQ ID NO: 513 - protein, 12 aa, Artificial Sequence
  Made in a lab
GGGGSCPPCPNS

SEQ ID NO: 514 - protein, 12 aa, Artificial Sequence
  Made in a lab
KADFLTPSIGNS

SEQ ID NO: 515 - protein, 12 aa, Artificial Sequence
  Made in a lab
QMNSELSVLANS

SEQ ID NO: 516 - protein, 13 aa, Artificial Sequence
  Made in a lab
EPKSCDKTHTCCP

SEQ ID NO: 517 - protein, 13 aa, Artificial Sequence
  Made in a lab
EPKSCDKTCPPCP

SEQ ID NO: 518 - protein, 13 aa, Artificial Sequence
  Made in a lab
GGGGSGGGGSGNS

SEQ ID NO: 519 - protein, 13 aa, Artificial Sequence
  Made in a lab
GCPPCPGGGGSNS

SEQ ID NO: 520 - protein, 13 aa, Artificial Sequence
  Made in a lab
GGGGSCPPCPGNS

SEQ ID NO: 521 - protein, 13 aa, Artificial Sequence
  Made in a lab
GCPPCPGGGGSNS

SEQ ID NO: 522 - protein, 13 aa, Artificial Sequence
  Made in a lab
GGGASCPPCPGNS

SEQ ID NO: 523 - protein, 13 aa, Artificial Sequence
  Made in a lab
GGGASCPPCAGNS

SEQ ID NO: 524 - protein, 13 aa, Artificial Sequence
  Made in a lab
GGGASCPPCAGNS

SEQ ID NO: 525 - protein, 15 aa, Artificial Sequence
  Made in a lab
NYGGGGSGGGGSGNS

SEQ ID NO: 526 - protein, 15 aa, Artificial Sequence
  Made in a lab
LSVKADFLTPSIGNS

SEQ ID NO: 527 - protein, 15 aa, Artificial Sequence
  Made in a lab
LSVLANFSQPEIGNS

SEQ ID NO: 528 - protein, 15 aa, Artificial Sequence
  Made in a lab
LKIQERVSKPKISNS

SEQ ID NO: 529 - protein, 15 aa, Artificial Sequence
  Made in a lab
LDVSERPFPPHIQNS

SEQ ID NO: 530 - protein, 15 aa, Artificial Sequence
  Made in a lab
REQLAEVTLSLKANS

SEQ ID NO: 531 - protein, 15 aa, Artificial Sequence
  Made in a lab
RIHQMNSELSVLANS

SEQ ID NO: 532 - protein, 15 aa, Artificial Sequence
  Made in a lab
DTKGKNVLEKIFSNS

SEQ ID NO: 533 - protein, 15 aa, Artificial Sequence
  Made in a lab
LPPETQESQEVTLNS

SEQ ID NO: 534 - protein, 15 aa, Artificial Sequence
  Made in a lab
RIHLNVSERPFPPNS

SEQ ID NO: 535 - protein, 15 aa, Artificial Sequence
  Made in a lab
LSVKADFLTPSIGNS

SEQ ID NO: 536 - protein, 15 aa, Artificial Sequence
  Made in a lab
LSVLANFSQPEIGNS

SEQ ID NO: 537 - protein, 15 aa, Artificial Sequence
  Made in a lab
LSVLANFSQPEIGNS

SEQ ID NO: 538 - protein, 15 aa, Artificial Sequence
  Made in a lab
RIHQMNSELSVLANS

SEQ ID NO: 539 - protein, 15 aa, Artificial Sequence
  Made in a lab
KPFFTCGSADTCPNS

SEQ ID NO: 540 - protein, 15 aa, Artificial Sequence
  Made in a lab
KPFFTCGSADTCPNS

SEQ ID NO: 541 - protein, 15 aa, Artificial Sequence
  Made in a lab
QYNCPGQYTFSMPNS

SEQ ID NO: 542 - protein, 15 aa, Artificial Sequence
  Made in a lab
GGGGSGGGGSGGGGS

SEQ ID NO: 543 - protein, 15 aa, Artificial Sequence
  Made in a lab
EPKSSDKTHTCPPCP

SEQ ID NO: 544 - protein, 15 aa, Artificial Sequence
  Made in a lab
EPKSSDKTHTCPPCS

SEQ ID NO: 545 - protein, 15 aa, Artificial Sequence
  Made in a lab
EPKSCDKTHTSPPCS

SEQ ID NO: 546 - protein, 15 aa, Artificial Sequence
  Made in a lab
EPKSCDKTHTCPPCP

SEQ ID NO: 547 - protein, 15 aa, Artificial Sequence
  Made in a lab
EPKSCDKTHTSPPSS

SEQ ID NO: 548 - protein, 15 aa, Artificial Sequence
  Made in a lab
PRGPTIKPCPPCKCP

SEQ ID NO: 549 - protein, 15 aa, Artificial Sequence
  Made in a lab
EPKSSDKTHTSPPSS

SEQ ID NO: 550 - protein, 15 aa, Artificial Sequence
  Made in a lab
EPKSSDKTHTSPPCS

SEQ ID NO: 551 - protein, 15 aa, Artificial Sequence
  Made in a lab
EPKSSDKTHTCPPSS

SEQ ID NO: 552 - protein, 15 aa, Artificial Sequence
  Made in a lab
EPKSCDKTHTSPPCP

SEQ ID NO: 553 - protein, 15 aa, Artificial Sequence
  Made in a lab
EPKSSDKTHTSPPSP

SEQ ID NO: 554 - protein, 15 aa, Artificial Sequence
  Made in a lab
EPKSSDKTHTSPPCP

SEQ ID NO: 555 - protein, 15 aa, Artificial Sequence
  Made in a lab
EPKSCDKTHTSPPSP

SEQ ID NO: 556 - protein, 15 aa, Artificial Sequence
  Made in a lab
EPKSSDKTHTCPPSP

SEQ ID NO: 557 - protein, 15 aa, Artificial Sequence
  Made in a lab
GGGGCDKTHTCPPCP

SEQ ID NO: 558 - protein, 15 aa, Artificial Sequence
  Made in a lab
EPKSCGGGGGCPPCP

SEQ ID NO: 559 - protein, 15 aa, Artificial Sequence
  Made in a lab
EPKSCDKTHTCGGCP

SEQ ID NO: 560 - protein, 15 aa, Artificial Sequence
  Made in a lab
EPKSCDKTHTCPPCG

SEQ ID NO: 561 - protein, 15 aa, Artificial Sequence
  Made in a lab
EPKSCDKTHTCPPSP

SEQ ID NO: 562 - protein, 15 aa, Artificial Sequence
  Made in a lab
EPKSCDKCHTCPPCP

SEQ ID NO: 563 - protein, 15 aa, Artificial Sequence
  Made in a lab
EPKSCDKTCCCPPCP

SEQ ID NO: 564 - protein, 15 aa, Artificial Sequence
  Made in a lab
EPKSCPPPPPCPPCP

SEQ ID NO: 565 - protein, 15 aa, Artificial Sequence
  Made in a lab
PPPPCDKTHTCPPCP

SEQ ID NO: 566 - protein, 15 aa, Artificial Sequence
  Made in a lab
EPKSCDKTHTCWWCP

SEQ ID NO: 567 - protein, 15 aa, Artificial Sequence
  Made in a lab
EPKSCDWWHTCPPCP

SEQ ID NO: 568 - protein, 15 aa, Artificial Sequence
  Made in a lab
EPKCSDKTHTCPPCP

SEQ ID NO: 569 - protein, 15 aa, Artificial Sequence
  Made in a lab
EPKSDCKTHTCPPCP

SEQ ID NO: 570 - protein, 15 aa, Artificial Sequence
  Made in a lab
EPKSDCWWHTCPPCP

SEQ ID NO: 571 - protein, 15 aa, Artificial Sequence
  Made in a lab
EPKSCDFFHTCPPCP

SEQ ID NO: 572 - protein, 15 aa, Artificial Sequence
  Made in a lab
EPKSCDWWWTCPPCP

SEQ ID NO: 573 - protein, 15 aa, Artificial Sequence
  Made in a lab
EPKSCWWTHTCPPCP

SEQ ID NO: 574 - protein, 15 aa, Artificial Sequence
  Made in a lab
EPWWCDKTHTCPPCP

SEQ ID NO: 575 - protein, 16 aa, Artificial Sequence
  Made in a lab
SQPEIVPISCPPCPNS

SEQ ID NO: 576 - protein, 17 aa, Artificial Sequence
  Made in a lab
TGEPKSSDKTHTCPPCP

SEQ ID NO: 577 - protein, 17 aa, Artificial Sequence
  Made in a lab
EPKSTDKTHTCPPCPNS

SEQ ID NO: 578 - protein, 17 aa, Artificial Sequence
  Made in a lab
EPKSTDKTHTSPPSPNS

SEQ ID NO: 579 - protein, 17 aa, Artificial Sequence
  Made in a lab
EPKSTDKTHTCPPCPNS

SEQ ID NO: 580 - protein, 17 aa, Artificial Sequence
  Made in a lab
EPKSSDKTHTCPPCPNS

SEQ ID NO: 581 - protein, 18 aa, Artificial Sequence
  Made in a lab
EPKSCDKTHTCPGGGPCP

SEQ ID NO: 582 - protein, 18 aa, Artificial Sequence
  Made in a lab
EPKSCDGGGKTHTCPPCP

SEQ ID NO: 583 - protein, 18 aa, Artificial Sequence
  Made in a lab
EPKSCDPPPKTHTCPPCP

SEQ ID NO: 584 - protein, 18 aa, Artificial Sequence
  Made in a lab
EPKSCDKTHTCPPPPPCP

SEQ ID NO: 585 - protein, 18 aa, Artificial Sequence
  Made in a lab
GGGGSGGGGSGGGGSGNS

SEQ ID NO: 586 - protein, 19 aa, Artificial Sequence
  Made in a lab
NYGGGGSGGGGSGGGGSNS

SEQ ID NO: 587 - protein, 19 aa, Artificial Sequence
  Made in a lab
LSVKADFLTPSISPPCPNS

SEQ ID NO: 588 - protein, 19 aa, Artificial Sequence
  Made in a lab
SVLANFSQPEISCPPCPNS

SEQ ID NO: 589 - protein, 20 aa, Artificial Sequence
  Made in a lab
GQRHNNSSLNTRTQKARHSP

SEQ ID NO: 590 - protein, 20 aa, Artificial Sequence
  Made in a lab
LSVLANFSQPEISCPPCPNS

SEQ ID NO: 591 - protein, 20 aa, Artificial Sequence
  Made in a lab
LKIQERVSKPKISCPPCPNS

SEQ ID NO: 592 - protein, 20 aa, Artificial Sequence
  Made in a lab
REQLAEVTLSLKACPPCPNS

SEQ ID NO: 593 - protein, 20 aa, Artificial Sequence
  Made in a lab
RIHQMNSELSVLACPPCPNS

SEQ ID NO: 594 - protein, 20 aa, Artificial Sequence
  Made in a lab
RIHLNVSERPFPPCPPCPNS

SEQ ID NO: 595 - protein, 20 aa, Artificial Sequence
  Made in a lab
NSLFNQEVQIPLTESYCPNS

SEQ ID NO: 596 - protein, 20 aa, Artificial Sequence
  Made in a lab
EEEEDEEDEEDEEEEEDGNS

SEQ ID NO: 597 - protein, 21 aa, Artificial Sequence
  Made in a lab
LDVSERPFPPHIQSCPPCPNS

SEQ ID NO: 598 - protein, 21 aa, Artificial Sequence
  Made in a lab
DTKGKNVLEKIFDSCPPCPNS

SEQ ID NO: 599 - protein, 21 aa, Artificial Sequence
  Made in a lab
LPPETQESQEVTLSCPPCPNS

SEQ ID NO: 600 - protein, 21 aa, Artificial Sequence
  Made in a lab
EPAFTPGPNIELQKDSDCPNS

SEQ ID NO: 601 - protein, 21 aa, Artificial Sequence
  Made in a lab
QRHNNSSLNTRTQKARHCPNS

SEQ ID NO: 602 - protein, 21 aa, Artificial Sequence
  Made in a lab
QRHNNSSLNTRTQKARHSPNS

SEQ ID NO: 603 - protein, 36 aa, Artificial Sequence
  Made in a lab
NYGGGGSGGGGSGGGGSNYGGGGSGGGGSGGGGSNS

SEQ ID NO: 604 - protein, 122 aa, Artificial Sequence
  Made in a lab
RTRYLQVSQQLQQTNRVLEVTNSSLRQQLRLKITQLGQSAEDLQGSRRELAQSQEALQVEQRAHQAAEGQLQACQADRQKTKETLQSEEQQRRALEQKLSNMENRLKPFFTCGSADTCCPNS

SEQ ID NO: 605 - skipped

SEQ ID NO: 606 - protein, 647 aa, Artificial Sequence
  Made in a lab
MDFQVQIFSFLLISASVIIARGVDPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAGGGSDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQMDQPRGPTIKPCPPCKCPAPNLLGGSSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 607 - skipped

SEQ ID NO: 608 - protein, 758 aa, Artificial Sequence [sequence omitted]

SEQ ID NO: 609-670 - skipped (62 entries)

SEQ ID NO: 671 - protein, 503 aa, Artificial Sequence
  Made in a lab
MDFQVQIFSFLLISASVIMSRGEVQLLESGGGLVQPGGSLRLSCAASGFTFSFYQMHWVRQAPGKGLEWVSGIYPSGGYTKYADSVKGRFTISRDNSKNTLYLQMNSLRAEDTAVYYCARDLGYGSSWYYFDYWGQGTLVTVSSGGGGSGGGGSGGGGSQSALTQPASVSGSPGQSITISCSGTDSDVGGYNHVSWYQQHPGKAPKLIIYDVDHRPSGISNRFSGSKSGNTASLTISGLQAEDEADYYCSSYRSGSTYVFGTGTKVTVLTGEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 672 - protein, 497 aa, Artificial Sequence [sequence omitted]

SEQ ID NO: 673 - protein, 499 aa, Artificial Sequence
  Made in a lab
MDFQVQIFSFLLISASVIMSRGEVQLLESGGGLVQPGGSLRLSCAASGFTFSHYSMGWVRQAPGKGLEWVSSIYPSGGSTLYADSVKGRFTISRDNSKNTLYLQMNSLRAEDTAVYYCARWGVGATFDYWGQGTLVTVSSGGGGSGGGGSGGGGSQDIQMTQSPGTLSLSPGERATLSCRASQSVSSSYLAWYQQKPGQAPRLLIYGASSRATGIPDRFSGSGSGTDFTLTISRLEPEDFAVYYCQQYGSSPLWTFGQGTKVEIKTGEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 674 - protein, 497 aa, Artificial Sequence
  Made in a lab
MDFQVQIFSFLLISASVIMSRGEVQLLESGGGLVQPGGSLRLSCAASGFTFSHYMMAWVRQAPGKGLEWVSYIYPSGGWTSYADSVKGRFTISRDNSKNTLYLQMNSLRAEDTAVYYCARFDYTIGFDFWGQGTLVTVSSGGGGSGGGGSGGGGSQDIQMTQSPSSLSASVGDRVTITCRASQSISSYLNWYQQKPGKAPKLLIYAASSLQSGVPSRFSGSGSGTDFTLTISSLQPEDFATYYCQQSYSTPRTFGQGTKVEIKTGEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 675 - protein, 500 aa, Artificial Sequence
  Made in a lab
MDFQVQIFSFLLISASVIMSRGEVQLLESGGGLVQPGGSLRLSCAASGFTFSWYNMLWVRQAPGKGLEWVSSISPSGGYTVYADSVKGRFTISRDNSKNTLYLQMNSLRAEDTAVYYCARDRGGSPFRPDAFDIWGQGTMVTVSSGGGGSGGGGSGGGGSQSALTQPPSVSVSPGQTARITCSGDKLGEKYVSWYQQRSGQSPLLVISQDTKRPSGIPERFSGSNSGNTATLTISGAQAADEADYYCQTWDSDTYVFGSGSTVTVLTGEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 676 - protein, 501 aa, Artificial Sequence
  Made in a lab
MDFQVQIFSFLLISASVIMSRGEVQLLESGGGLVQPGGSLRLSCAASGFTFSGYVMGWVRQAPGKGLEWVSSISPSGGYTLYADSVKGRFTISRDNSKNTLYLQMNSLRAEDTAVYYCARAFSTRWYWGAFDIWGQGTMVTVSSGGGGSGGGGSGGGGSQDIQMTQSPATLSLSPGERATLSCRASQSVRSYLAWYQQKPGQAPRLLIYDASNRATGIPARFSGSGSGTDFTLTITSLQPEDIATYYCQQYDNLPLTFGGGTKVEIKTGEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 677 - protein, 504 aa, Artificial Sequence
  Made in a lab
MDFQVQIFSFLLISASVIMSRGEVQLLESGGGLVQPGGSLRLSCAASGFTFSIYHMNWVRQAPGKGLEWVSGISSSGGRTNYADSVKGRFTISRDNSKNTLYLQMNSLRAEDTAVYYCASSYRAAGWVDYYYGMDVWGQGTTVTVSSGGGGSGGGGSGGGGSQDIQMTQSPSSLSAFVGDRVTITCRTSQSVATYVNWYQQKPGEGPKLLIYAASSLQSGVPSRFRGSGSGTDFTLTIGSLQPEDFATYYCQQSYSVPITFGQGTRLDIKTGEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 678 - protein, 496 aa, Artificial Sequence
  Made in a lab
MDFQVQIFSFLLISASVIMSRGEVQLLESGGGLVQPGGSLRLSCAASGFTFSDYPMQWVRQAPGKGLEWVSWIGPSGGWTVYADSVKGRFTISRDNSKNTLYLQMNSLRAEDTAVYYCARDDGIAGFLWGQGTLVTVSSGGGGSGGGGSGGGGSQDIQMTQSPLSLSASVGDRVTITCQASRDISIYLNWYQQKPGKVPKLLIFGASTLRSGVPSRFSGSGSGTDFTLTITSLQPEDVATYYCQKYNSAPYTFGPGTKVEIRTGEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 679 - protein, 504 aa, Artificial Sequence
  Made in a lab
MDFQVQIFSFLLISASVIMSRGEVQLLESGGGLVQPGGSLRLSCAASGFTFSWYLMHWVRQAPGKGLEWVSGIWPSGGHTLYADSVKGRFTISRDNSKNTLYLQMNSLRAEDTAVYYCAREPLLWFGELSYNWFDPWGQGTLVTVSSGGGGSGGGGSGGGGSQDIQMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKVEIKTGEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 680 - protein, 503 aa, Artificial Sequence
  Made in a lab
MDFQVQIFSFLLISASVIMSRGEVQLLESGGGLVQPGGSLRLSCAASGFTFSFYGMAWVRQAPGKGLEWVSSIYPSGGYTDYADSVKGRFTISRDNSKNTLYLQMNSLRAEDTAVYYCARSAGGWIGGGAFDIWGQGTMVTVSSGGGGSGGGGSGGGGSQSELTQPPSASGTPGQRVTISCSGSSSNIGSNTVNWYQQLPGTAPKLLIYSNNQRPSGVPDRFSGSKSGTSASLAISGLQSEDEADYYCAAWDDSLNGYVFGIGTKVTVLTGEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 681 - protein, 506 aa, Artificial Sequence
  Made in a lab
MDFQVQIFSFLLISASVIMSRGEVQLLESGGGLVQPGGSLRLSCAASGFTFSWYSMWWVRQAPGKGLEWVSYIVPSGGETIYADSVKGRFTISRDNSKNTLYLQMNSLRAEDTAVYYCAKNLGEGFWSDYYPPLDYWGQGTLVTVSSGGGGSGGGGSGGGGSQSVLTQPPSASGTPGQRVTISCSGSSSNIGGNAVNWYQQLPGTAPKLVLYSSNHRPSGVPDRFSGSRSGTSASLAISGLQSQDEADYYCAAWDDSLNGPLFGGGTKLTVLTGEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 682 - protein, 497 aa, Artificial Sequence
  Made in a lab
MDFQVQIFSFLLISASVIMSRGEVQLLESGGGLVQPGGSLRLSCAASGFTFSHYGMHWVRQAPGKGLEWVSSIYPGMTQYADSVKGRFTISRDNSKNTLYLQMNSLRAEDTAVYYCARDRGSGIDAFDIWGQGTMVTVSSGGGGSGGGGSGGGGSQSELTQPPSVSVSPGQTATVSCSGEKLGDRYAWWYQQKAGQSPVLVIYQDTKRPSGVPDRFSGSNSGTSASLVISGPQSEDEADYYCAAWDDSLKGVVFGGGSKLTVLTGEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 683 - protein, 506 aa, Artificial Sequence
  Made in a lab
MDFQVQIFSFLLISASVIMSRGEVQLLESGGGLVQPGGSLRLSCAASGFTFSWYDMLWVRQAPGKGLEWVSVISPSGGRTFYADSVKGRFTISRDNSKNTLYLQMNSLRAEDTAVYYCAVTRSMYSSSWYGAPPTHWGQGTLVTVSSGGGGSGGGGSGGGGSQSELTQPPSASGTPGQRVTISCSGRSSNIGSNSVNWYQQLPGTAPKLLIYSNNHRPSGVPDRFSGSKSGTSASLAISGLQSEDEADYYCAAWDDSLYQGVFGGGTKLTVLTGEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 684 - protein, 494 aa, Artificial Sequence
  Made in a lab
MDFQVQIFSFLLISASVIMSRGEVQLLESGGGLVQPGGSLRLSCAASGFTFSSYWMHWVRQAPGKGLEWVSSIYPSGGSTEYADSVKGRFTISRDNSKNTLYLQMNSLRAEDTAVYYCAKGIWFDPWGQGTLVTVSSGGGGSGGGGSGGGGSQDIQMTQSPATLSASVGDRVTITCRSSQRISGYLNWYQQQPGKAPKLLIYAASTLQSGVPSRISGSGSGTDFSLIISSLQPEDVATYYCQQSYSPPFTFGGGTKVEIKTGEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 685 - protein, 501 aa, Artificial Sequence
  Made in a lab
MDFQVQIFSFLLISASVIMSRGEVQLLESGGGLVQPGGSLRLSCAASGFTFSTYEMNWVRQAPGKGLEWVSGIVPSGGVTYYADSVKGRFTISRDNSKNTLYLQMNSLRAEDTAVYYCARGPYSYGHDYGMDVWGQGTTVTVSSGGGGSGGGGSGGGGSQDIQMTQSPSTLSASVGDRVTITCRASQSISSWLAWYQQKPGKAPKLLIYKASNLESEVPSRFSGSGSGTEFTLTISSLQPDDFATYYCQQYNAYPFIFGGGTKVEIKTGEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 686 - protein, 501 aa, Artificial Sequence
  Made in a lab
MDFQVQIFSFLLISASVIMSRGEVQLLESGGGLVQPGGSLRLSCAASGFTFSSYTMHWVRQAPGKGLEWVSSISPSGGMTFYADSVKGRFTISRDNSKNTLYLQMNSLRAEDTAVYYCARTYDFWSGYFDYWGQGTLVTVSSGGGGSGGGGSGGGGSQSELTQPPSVSEAPRQRVTVSCSGSPSNIGSNSVNWYQQLPGKAPKVVIYYDDLVPSGVSDRFSGSKSGTSASLAISGLQSEDEADYYCAAWDDRLNGWVFGGGTKLTVLTGEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 687 - protein, 499 aa, Artificial Sequence
  Made in a lab
MDFQVQIFSFLLISASVIMSRGEVQLLESGGGLVQPGGSLRLSCAASGFTFSWYWMTWVRQAPGKGLEWVSSIPPSGGHTSYADSVKGRFTISRDNSKNTLYLQMNSLRAEDTAVYYCAKSSGWYEDYFDYWGQGTLVTVSSGGGGSGGGGSGGGGSQDIQMTQSPSSLSASVGDRVTITCQASQDITNYLNWYLHKPGKAPKLLIYDASNLQTGVPSRFSGSGSGTDFTFTITSLQPEDFGTYYCQQYDTLHPSFGPGTTVDIKTGEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 688 - protein, 497 aa, Artificial Sequence
  Made in a lab
MDFQVQIFSFLLISASVIMSRGEVQLLESGGGLVQPGGSLRLSCAASGFTFSAYSMVWVRQAPGKGLEWVSYIYPSGGITTYADSVKGRFTISRDNSKNTLYLQMNSLRAEDTAVYYCAREGQVFDIWGQGTTVTVSSGGGGSGGGGSGGGGSQDIQMTQSPATLSLSPGERATLSCRASQSVTTYLAWYQQRPGQAPRLLIYDASNRATGIPARFSGSGSGTDFTLTISSVEPEDYAVYYCQQRSNWPPSITFGQGTRLESKTGEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 689 - protein, 499 aa, Artificial Sequence
  Made in a lab
MDFQVQIFSFLLISASVIMSRGEVQLLESGGGLVQPGGSLRLSCAASGFTFSNYAMGWVRQAPGKGLEWVSRIVPSGGMTSYADSVKGRFTISRDNSKNTLYLQMNSLRAEDTAVYYCTTDDFWSGMDVWGQGTTVTVSSGGGGSGGGGSGGGGSQSALTQPASVSGSPGQSITISCTGTSSDVGSYNLVSWYQQHPGKAPKVMIYEGSKRPSGVSNRFSGSKSGNTASLTISGLQAEDEADYYCCSYAGRSTYVFGTGTKVTVLTGEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 690 - protein, 506 aa, Artificial Sequence
  Made in a lab
MDFQVQIFSFLLISASVIMSRGEVQLLESGGGLVQPGGSLRLSCAASGFTFSAYTMHWVRQAPGKGLEWVSSIYPSGGTTPYADSVKGRFTISRDNSKNTLYLQMNSLRAEDTATYYCARVVNIDFWSGYNMRSAFDIWGQGTMVTVSSGGGGSGGGGSGGGGSQDIQMTQSPSSVSASVGDTVTITCRASQGVSDWLAWYQQKPGKAPNLLIYGASSLQTGVPSRFSGGGSGTDFTLTISSLQPEDFATYYCQQAHSFPFTFGGGTRVEIKTGEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 691 - protein, 500 aa, Artificial Sequence
  Made in a lab
MDFQVQIFSFLLISASVIMSRGEVQLLESGGGLVQPGGSLRLSCAASGFTFSEYGMSWVRQAPGKGLEWVSSIRSSGGWTKYADSVKGRFTISRDNSKNTLYLQMNSLRAEDTAVYYCAKGLGATSGEFDYWGQGTLVTVSSGGGGSGGGGSGGGGSQDIQMTQSPGTLSLSPGERATLSCRASQSVSGNYLAWYQQKLGQAPRLLIYGASSRATGTPDRFSGSGSGTEFTLTISSLQPEDFATYYCLQHNSYPLTFGEGTKVEIKTGEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 692 - protein, 502 aa, Artificial Sequence
  Made in a lab
MDFQVQIFSFLLISASVIMSRGEVQLLESGGGLVQPGGSLRLSCAASGFTFSQYFMMWVRQAPGKGLEWVSSISPSGGYTQYADSVKGRFTISRDNSKNTLYLQMNSLRAEDTAVYYCAKTGDYTRYYSYGMDVWGQGTLVTVSSGGGGSGGGGSGGGGSQDIQMTQSPSSLSASVGDRVTITCRASQSISSYLNWYQQKPGKAPKLLIYAASSLQSGVPSRFSGSGSGTDFTLIISSLQPEDFATYYCQQSYSTPLTFGGGTKVEIKTGEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 693 - protein, 503 aa, Artificial Sequence
  Made in a lab
MDFQVQIFSFLLISASVIMSRGEVQLLESGGGLVQPGGSLRLSCAASGFTFSGYSMAWVRQAPGKGLEWVSGIYSSGGWTWYADSVKGRFTISRDNSKNTLYLQMNSLRAEDTAVYYCTRDQSFSYDSSLDAFDIWGQGTMVTVSSGGGGSGGGGSGGGGSQDIQMTQSPSSLSASVGDRVTITCRASQSINNYLNWYQQKPGKAPKLLIYTASTLHSGVPSRFSGSGSGTDFTLTISNLQPEDFATYYCQQSYATLRTFGQGTKVEIKTGEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 694 - protein, 505 aa, Artificial Sequence
  Made in a lab
MDFQVQIFSFLLISASVIMSRGEVQLLESGGGLVQPGGSLRLSCAASGFTFSLYGMHWVRQAPGKGLEWVSSIVPSGGLTRYADSVKGRFTISRDNSKNTLYLQMNSLRAEDTATYYCARLAYYDFWSGRDAFDIWGQGTMVTVSSGGGGSGGGGSGGGGSQSVLTQPASVSGSPGQSITISCSGSSSDVGHYDYVSWYQQHPGKAPKLMIYDVSNRPSGVSNRFSGSKSGNTASLTISGLQADDEAEYYCSSYTSSGTRVFGTGTKVTVLTGEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKAYACAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 695 - protein, 257 aa, Human
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

SEQ ID NO: 696 - protein, 211 aa, Human
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTT

SEQ ID NO: 697-760 - skipped (64 entries)

SEQ ID NO: 761 - DNA, 1532 bp, Artificial Sequence
  Made in a lab
aagcttgccgccatggattttcaagtgcagattttcagcttcctgctaatcagtgcttcagtcataatgtcgcgaggagaagttcaattgttagagtctggtggcggtcttgttcagcctggtggttctttacgtctttcttgcgctgcttccggattcactttctctttttaccagatgcattgggttcgccaagctcctggtaaaggtttggagtgggtttctggtatctatccttctggtggctatactaagtatgctgactccgttaaaggtcgcttcactatctctagagacaactctaagaatactctctacttgcagatgaacagcttaagggctgaggacactgcagtctactattgtgcgagagatctagggtacggcagcagctggtactactttgactactggggccagggaaccctggtcaccgtctcaagcggtggcggcggttcggggggtggcggaagtggaggtggagggagtcagagcgctttgactcagcctgcctccgtgtctgggtctcctggacagtcgatcaccatctcctgcagtggaaccgacagtgacgttggtggttataaccatgtctcctggtaccaacaacacccaggcaaggcccccaaactcataatttatgatgtcgatcatcggccctcagggatctctaaccgtttctctggctccaagtctggcaacacggcctccctgaccatctctgggctccaggctgaggacgaggctgattattactgcagctcatatagaagcggtagcacttatgtcttcggaactgggaccaaggtcaccgtcctaaccggtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagccggtggcagcaggggaacgtcttctcatgctccgtgatgcatgaggctctgcacaaccactacacgcagaagagcctctccctgtctccgggtaaatgagcggccgc

SEQ ID NO: 762 - DNA, 1514 bp, Artificial Sequence
  Made in a lab
aagcttgccgccatggattttcaagtgcagattttcagcttcctgctaatcagtgcttcagtcataatgtcgcgaggagaagttcaattgttagagtctggtggcggtcttgttcagcctggtggttctttacgtctttcttgcgctgcttccggattcactttctctgagtaccctatgaattgggttcgccaagctcctggtaaaggtttggagtgggtttctggtatctattcttctggtggccttactaagtatgctgactccgttaaaggtcgcttcactatctctagagacaactctaagaatactctctacttgcagatgaacagcttaagggctgaggacacggccgtgtattactgtgcgagagatcactacggtgactacgcatttgactactggggccagggaaccctggtcaccgtctcaagcggtggcggcggttcggggggtggcggaagtggaggtggagggagtcaagacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcatctagtttagaaagtggggtcccatcaaggttcagcggcagtggatctgggacagaattcactctcaccatcagcagcctgcagcctgatgattttgcaacttattactgccaacagtataatagttattggacgttcggccaagggaccaaggtggaaatcaaaaccggtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagccggtggcagcaggggaacgtcttctcatgctccgtgatgcatgaggctctgcacaaccactacacgcagaagagcctctccctgtctccgggtaaatgagcggccgc

SEQ ID NO: 763 - DNA, 1520 bp, Artificial Sequence
  Made in a lab
aagcttgccgccatggattttcaagtgcagattttcagcttcctgctaatcagtgcttcagtcataatgtcgcgaggagaagttcaattgttagagtctggtggcggtcttgttcagcctggtggttctttacgtctttcttgcgctgcttccggattcactttctctcattactctatgggttgggttcgccaagctcctggtaaaggtttggagtgggtttcttctatctatccttctggtggctctactctttatgctgactccgttaaaggtcgcttcactatctctagagacaactctaagaatactctctacttgcagatgaacagcttaagggctgaggacacggccgtgtattactgtgcgagatggggagtgggagctacgttcgactactggggccagggcaccctggtcaccgtctcaagcggtggcggcggttcggggggtggcggaagtggaggtggagggagtcaagacatccagatgacccagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctctctggacgttcggccaagggaccaaggtggaaatcaaaaccggtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagccggtggcagcaggggaacgtcttctcatgctccgtgatgcatgaggctctgcacaaccactacacgcagaagagcctctccctgtctccgggtaaatgagcggccgc

SEQ ID NO: 764 - DNA, 1514 bp, Artificial Sequence
  Made in a lab
aagcttgccgccatggattttcaagtgcagattttcagcttcctgctaatcagtgcttcagtcataatgtcgcgaggagaagttcaattgttagagtctggtggcggtcttgttcagcctggtggttctttacgtctttcttgcgctgcttccggattcactttctctcattacatgatggcttgggttcgccaagctcctggtaaaggtttggagtgggtttcttatatctatccttctggtggctggacttcttatgctgactccgttaaaggtcgcttcactatctctagagacaactctaagaatactctctacttgcagatgaacagcttaagggctgaggacacggccgtgtattactgtgcgagattcgactatactataggttttgacttctggggccagggaaccctggtcaccgtctcaagcggtggcggcggttcggggggtggcggaagtggaggtggagggagtcaagacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctcggacgttcggccaagggaccaaggtggaaatcaaaaccggtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagccggtggcagcaggggaacgtcttctcatgctccgtgatgcatgaggctctgcacaaccactacacgcagaagagcctctccctgtctccgggtaaatgagcggccgc

SEQ ID NO: 765 - DNA, 1515 bp, Artificial Sequence
  Made in a lab
aagcttgccgccatggattttcaagtgcagattttcagcttcctgctaatcagtgcttcagtcataatgtcgcgaggagaagttcaattgttagagtctggtggcggtcttgttcagcctggtggttctttacgtctttcttgcgctgcttccggattcactttctcttggtacaatatgctttgggttcgccaagctcctggtaaaggtttggagtgggtttcttctatctctccttctggtggctatactgtttatgctgactccgttaaaggtcgcttcactatctctagagacaactctaagaatactctctacttgcagatgaacagcttaagggctgaggacacggccgtgtattactgtgcacgggaccgtggtgggagccccttccggcctgatgcttttgatatctggggccaagggacaatggtcaccgtctcaagcggtggcggcggttcggggggtggcggaagtggaggtggagggagtcagagcgctttgactcagccaccctcagtgtccgtgtccccaggacagacagccaggatcacctgctctggagataagttgggggaaaaatatgtttcctggtatcagcagcggtcaggccagtcccctctactggtcatttctcaagataccaagcggccctcagggatccctgagcgattctctggctccaactctgggaacacagccactctgaccatcagcggggcccaggctgcagatgaggctgactattactgtcagacgtgggacagcgacacttatgtcttcggaagtgggagcacggtcaccgtcctgaccggtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagccggtggcagcaggggaacgtcttctcatgctccgtgatgcatgaggctctgcacaaccactacacgcagaagagcctctccctgtctccgggtaaatga

SEQ ID NO: 766 - DNA, 1526 bp, Artificial Sequence
  Made in a lab
aagcttgccgccatggattttcaagtgcagattttcagcttcctgctaatcagtgcttcagtcataatgtcgcgaggagaagttcaattgttagagtctggtggcggtcttgttcagcctggtggttctttacgtctttcttgcgctgcttccggattcactttctctggttacgttatgggttgggttcgccaagctcctggtaaaggtttggagtgggtttcttctatctctccttctggtggctatactctttatgctgactccgttaaaggtcgcttcactatctctagagacaactctaagaatactctctacttgcagatgaacagcttaagggctgaggacacggctgtgtattactgtgcgagagcatttagcaccaggtggtactggggtgcttttgatatctggggccaagggacaatggtcaccgtctcaagcggtggcggcggttcggggggtggcggaagtggaggtggagggagtcaagacatccagatgacccagtctccagccaccctgtctttgtctccaggggaaagagccaccctctcctgcagggccagtcagagtgttaggagctacttagcctggtaccaacagaaacctggccaggctcccaggctcctcatctatgatgcatccaacagggccactggcatcccagccaggttcagtggcagtgggtctgggacagacttcactctcaccatcaccagcctgcagcctgaagatattgcaacatattactgtcaacagtatgataacctcccgctcactttcggcggagggaccaaggtggagatcaaaaccggtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagccggtggcagcaggggaacgtcttctcatgctccgtgatgcatgaggctctgcacaaccactacacgcagaagagcctctccctgtctccgggtaaatgagcggccgc

SEQ ID NO: 767 - DNA, 1527 bp, Artificial Sequence
  Made in a lab
aagcttgccgccatggattttcaagtgcagattttcagcttcctgctaatcagtgcttcagtcataatgtcgcgaggagaagttcaattgttagagtctggtggcggtcttgttcagcctggtggttctttacgtctttcttgcgctgcttccggattcactttctctatttaccatatgaattgggttcgccaagctcctggtaaaggtttggagtgggtttctggtatctcttcttctggtggccgtactaattatgctgactccgttaaaggtcgcttcactatctctagagacaactctaagaatactctctacttgcagatgaacagcttaagggctgaggacacggccgtgtattactgtgcgagttcctaccgagcagctggttgggtggactactactacggtatggacgtctggggccaagggaccacggtcaccgtctcaagcggtggcggcggttcggggggtggcggaagtggaggtggagggagtcaagacatccagatgacccagtctccatcctccctgtctgcatttgtaggagacagagtcaccatcacttgccggacaagtcagagcgttgccacctatgtaaattggtatcagcagaaaccaggagaaggccctaagctcctgatctatgctgcatccagtttacaaagtggggtcccatcgaggttcagaggcagtggatctgggacagatttcactctcaccatcggcagtctgcaacctgaagattttgcaacttactactgtcaacagagttacagtgtgccaatcaccttcggccaagggacacgactggacattaaaaccggtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagccggtggcagcaggggaacgtcttctcatgctccgtgatgcatgaggctctgcacaaccactacacgcagaagagcctctccctgtctccgggtaaatga

SEQ ID NO: 768 - DNA, 1511 bp, Artificial Sequence
  Made in a lab
aagcttgccgccatggattttcaagtgcagattttcagcttcctgctaatcagtgcttcagtcataatgtcgcgaggagaagttcaattgttagagtctggtggcggtcttgttcagcctggtggttctttacgtctttcttgcgctgcttccggattcactttctctgattaccctatgcagtgggttcgccaagctcctggtaaaggtttggagtgggtttcttggatcggtccttctggtggctggactgtttatgctgactccgttaaaggtcgcttcactatctctagagacaactctaagaatactctctacttgcagatgaacagcttaagggctgaggacacggccgtgtattactgtgcgagggatgacggaattgctggattcctatggggccagggcaccctggtcaccgtctcaagcggtggcggcggttcggggggtggcggaagtggaggtggagggagtcaagacatccagatgacccagtctccactctccctgtctgcatctgtaggagacagagtcaccatcacttgccaggcgagtcgagacattagtatctatttaaattggtatcagcagaaaccagggaaagttcctaagctcctgatctttggtgcgtccactttgcgatcaggggtcccatctcggttcagtggcagtggatctgggacagatttcactctcaccatcaccagcctgcagcctgaagatgttgcaacttattactgtcaaaagtataacagtgccccgtacacttttggcccggggaccaaggtggagatcagaaccggtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagccggtggcagcaggggaacgtcttctcatgctccgtgatgcatgaggctctgcacaaccactacacgcagaagagcctctccctgtctccgggtaaatgagcggccgc

SEQ ID NO: 769 - DNA, 1535 bp, Artificial Sequence
  Made in a lab
aagcttgccgccatggattttcaagtgcagattttcagcttcctgctaatcagtgcttcagtcataatgtcgcgaggagaagttcaattgttagagtctggtggcggtcttgttcagcctggtggttctttacgtctttcttgcgctgcttccggattcactttctcttggtaccttatgcattgggttcgccaagctcctggtaaaggtttggagtgggtttctggtatctggccttctggtggccatactctttatgctgactccgttaaaggtcgcttcactatctctagagacaactctaagaatactctctacttgcagatgaacagcttaagggctgaggacacggccgtgtattactgtgcgagagagccgttactatggttcggggagttatcgtacaactggttcgacccctggggccagggcaccctggtcaccgtctcaagcggtggcggcggttcggggggtggcggaagtggaggtggagggagtcaagacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttccctctcactttcggcggagggaccaaggtggagatcaaaaccggtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagccggtggcagcaggggaacgtcttctcatgctccgtgatgcatgaggctctgcacaaccactacacgcagaagagcctctccctgtctccgggtaaatgagcggccgc

SEQ ID NO: 770 - DNA, 1533 bp, Artificial Sequence
  Made in a lab
aagcttgccgccatggattttcaagtgcagattttcagcttcctgctaatcagtgcttcagtcataatgtcgcgaggagaagttcaattgttagagtctggtggcggtcttgttcagcctggtggttctttacgtctttcttgcgctgcttccggattcactttctctttttacggtatggcttgggttcgccaagctcctggtaaaggtttggagtgggtttcttctatctatccttctggtggctatactgattatgctgactccgttaaaggtcgcttcactatctctagagacaactctaagaatactctctacttgcagatgaacagcttaagggctgaggacacggccgtgtattactgtgcgagatccgcgggaggctggatcggggggggtgcttttgatatctggggccaagggacaatggtcaccgtctcaagcggtggcggcggttcggggggtggcggaagtggaggtggagggagtcagagcgaattgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggttatgtcttcggaattgggaccaaggtcaccgtcctaaccggtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagccggtggcagcaggggaacgtcttctcatgctccgtgatgcatgaggctctgcacaaccactacacgcagaagagcctctccctgtctccgggtaaatgagcggccgca

SEQ ID NO: 771 - DNA, 1541 bp, Artificial Sequence
  Made in a lab
aagcttgccgccatggattttcaagtgcagattttcagcttcctgctaatcagtgcttcagtcataatgtcgcgaggagaagttcaattgttagagtctggtggcggtcttgttcagcctggtggttctttacgtctttcttgcgctgcttccggattcactttctcttggtactctatgtggtgggttcgccaagctcctggtaaaggtttggagtgggtttcttatatcgttccttctggtggcgagactatttatgctgactccgttaaaggtcgcttcactatctctagagacaactctaagaatactctctacttgcagatgaacagcttaagggctgaggacacggccgtgtattactgtgcgaaaaaccttggtgaaggattttggagtgattattaccctccgcttgactactggggccagggaaccctggtcaccgtctcaagcggtggcggcggttcggggggtggcggaagtggaggtggagggagtcagagcgtcttgactcagccaccctcagcgtctgggacccccgggcagagggtcaccatctcttgttctggaagcagctccaacatcggaggtaatgctgtaaactggtaccagcagctcccgggaacggcccccaaactcgtcctctatagtagtaatcaccggccctcaggggtccctgaccgattctctggctccaggtctggcacctcagcctccctggccatcagtgggctccagtctcaggatgaggctgattactactgtgcagcatgggatgacagcctgaatggtccgctcttcggcggagggaccaagctgaccgtcctaaccggtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagccggtggcagcaggggaacgtcttctcatgctccgtgatgcatgaggctctgcacaaccactacacgcagaagagcctctccctgtctccgggtaaatgagcggccgc

SEQ ID NO: 772 - DNA, 1514 bp, Artificial Sequence
  Made in a lab
aagcttgccgccatggattttcaagtgcagattttcagcttcctgctaatcagtgcttcagtcataatgtcgcgaggagaagttcaattgttagagtctggtggcggtcttgttcagcctggtggttctttacgtctttcttgcgctgcttccggattcactttctctcattacggtatgcattgggttcgccaagctcctggtaaaggtttggagtgggtttcttctatctatcctggcatgactcagtatgctgactccgttaaaggtcgcttcactatctctagagacaactctaagaatactctctacttgcagatgaacagcttaagggctgaggacacggccgtgtattactgtgcgagagatcggggttcggggattgatgcttttgatatctggggccaagggacaatggtcaccgtctcaagcggtggcggcggttcggggggtggcggaagtggaggtggagggagtcagagcgaattgactcagccaccctcagtgtccgtgtccccaggacagacggccaccgtcagctgctctggagaaaaattgggggatagatatgcctggtggtatcaacagaaggcaggccagtcccctgtgctggtcatctatcaagataccaagcggccctcaggggtccctgaccgattctctggctccaactctggcacctcagcctccctggtcatcagtggaccccagtctgaggatgaggctgattattactgtgcagcatgggatgacagcctgaagggtgtggtattcggcggagggtccaagctgaccgtcctaaccggtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagccggtggcagcaggggaacgtcttctcatgctccgtgatgcatgaggctctgcacaaccactacacgcagaagagcctctccctgtctccgggtaaatgagcggccgc

SEQ ID NO: 773 - DNA, 1541 bp, Artificial Sequence
  Made in a lab
aagcttgccgccatggattttcaagtgcagattttcagcttcctgctaatcagtgcttcagtcataatgtcgcgaggagaagttcaattgttagagtctggtggcggtcttgttcagcctggtggttctttacgtctttcctgcgctgcttccggattcactttctcttggtacgatatgctttgggttcgccaagctcctggtaaaggtttggagtgggtttctgttatctccccttctggtggccgtactttttatgctgactccgttaaaggtcgcttcactatctctagagacaactctaagaatactctctacttgcagatgaacagcttaagggctgaggacacggccgtgtattactgtgcggttactcgatcgatgtatagcagcagctggtacggggcccccccgactcactggggccagggcaccctggtcaccgtctcaagcggtggcggcggttcggggggtggcggaagtggaggtggagggagtcagagcgaattgactcagccaccctcagcgtctgggacccccgggcagagggtcaccatctcttgttctggaaggagctccaacatcggaagtaatagtgtaaactggtaccagcagctcccaggaacggcccccaaactcctcatctatagtaataatcaccggccctcaggggtccctgaccgattctctggctccaagtctggcacctcagcctccctggccatcagtgggctccagtctgaggatgaggctgattattactgtgcagcatgggatgacagcctgtatcaaggagtgttcggcggagggaccaagctgaccgtcctaaccggtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagccggtggcagcaggggaacgtcttctcatgctccgtgatgcatgaggctctgcacaaccactacacgcagaagagcctctccctgtctccgggtaaatgagcggccgc

SEQ ID NO: 774 - DNA, 1505 bp, Artificial Sequence
  Made in a lab
aagcttgccgccatggattttcaagtgcagattttcagcttcctgctaatcagtgcttcagtcataatgtcgcgaggagaagttcaattgttagagtctggtggcggtcttgttcagcctggtggttctttacgtctttcttgcgctgcttccggattcactttctcttcttactggatgcattgggttcgccaagctcctggtaaaggtttggagtgggtttcttctatctatccttctggtggctctactgagtatgctgactccgttaaaggtcgcttcactatctctagagacaactctaagaatactctctacttgcagatgaacagcttaagggctgaggacacggccgtgtattactgtgcgaaaggaatctggttcgacccctggggccagggaaccctggtcaccgtctcaagcggtggcggcggttcggggggtggcggaagtggaggtggagggagtcaagacatccagatgacccagtctccagccaccctgtctgcctctgtaggagacagggtcaccatcacttgccggtcaagtcagaggattagcggctatttaaattggtatcagcagcaaccagggaaagcccctaagctcctgatctatgctgcatccaccttacaaagtggggtcccatcaaggatcagtggcagtggatctgggacagatttcagtctcatcatcagcagtctgcaacctgaagatgttgcaacttattattgtcaacagagttacagtcccccgttcacttttggcggagggaccaaggtggagatcaaaaccggtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagccggtggcagcaggggaacgtcttctcatgctccgtgatgcatgaggctctgcacaaccactacacgcagaagagcctctccctgtctccgggtaaatgagcggccgc

SEQ ID NO: 775 - DNA, 1526 bp, Artificial Sequence
  Made in a lab
aagcttgccgccatggattttcaagtgcagattttcagcttcctgctaatcagtgcttcagtcataatgtcgcgaggagaagttcaattgttagagtctggtggcggtcttgttcagcctggtggttctttacgtctttcttgcgctgcttccggattcactttctctacttacgagatgaattgggttcgccaagctcctggtaaaggtttggagtgggtttctggtatcgttccttctggtggcgttacttattatgctgactccgttaaaggtcgcttcactatctctagagacaactctaagaatactctctacttgcagatgaacagcttaagggctgaggacacggccgtgtattactgtgcgaggggaccgtacagctatggtcacgactacggtatggacgtctggggccaagggaccacggtcaccgtctcaagcggtggcggcggttcggggggtggcggaagtggaggtggagggagtcaagacatccagatgacccagtctccttccaccctgtctgcatctgtaggagacagggtcaccatcacttgccgggccagtcagagtattagtagctggttggcctggtatcagcagaaaccagggaaagcccctaagctcctgatctataaggcgtctaatttagaaagtgaggtcccatcaaggttcagcggcagtggatctgggacagaattcactctcaccatcagcagcctgcagcctgatgattttgcaacttattactgccaacagtataatgcttatccattcattttcggcggagggaccaaggtggagatcaaaaccggtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagccggtggcagcaggggaacgtcttctcatgctccgtgatgcatgaggctctgcacaaccactacacgcagaagagcctctccctgtctccgggtaaatgagcggccgc

SEQ ID NO: 776 - DNA, 1526 bp, Artificial Sequence
  Made in a lab
aagcttgccgccatggattttcaagtgcagattttcagcttcctgctaatcagtgcttcagtcataatgtcgcgaggagaagttcaattgttagagtctggtggcggtcttgttcagcctggtggttctttacgtctttcttgcgctgcttccggattcactttctcttcttacactatgcattgggttcgccaagctcctggtaaaggtttggagtgggtttcttctatctctccttctggtggcatgactttttatgctgactccgttaaaggtcgcttcactatctctagagacaactctaagaatactctctacttgcagatgaacagcttaagggctgaggacactgcagtctactattgtgcgagaacttacgatttttggagtggttattttgactactggggccagggaactctggtcaccgtctcaagcggtggcggcggttcggggggtggcggaagtggaggtggagggagtcagagcgaattgactcagccaccctcggtatctgaagcccccaggcagagggtcaccgtctcctgttctggaagcccctccaacatcggaagtaattctgtaaactggtaccaacagctcccaggaaaggctcccaaagtcgtcatctattatgatgatctggtgccctcaggagtctctgaccgattctctggctccaagtctggcacttcagcctccctggccatcagtgggctccagtctgaggatgaggctgattattactgtgcagcatgggatgacaggctgaatggttgggtgttcggcggagggaccaagctgaccgtcctaaccggtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagccggtggcagcaggggaacgtcttctcatgctccgtgatgcatgaggctctgcacaaccactacacgcagaagagcctctccctgtctccgggtaaatgagcggccgc

SEQ ID NO: 777 - DNA, 1521 bp, Artificial Sequence
  Made in a lab
aagcttgccgccatggattttcaagtgcagattttcagcttcctgctaatcagtgcttcagtcataatgtcgcgaggagaagttcaattgttagagtctggtggcggtcttgttcagcctggtggttctttacgtctttcttgcgctgcttccggattcactttctcttggtactggatgacttgggttcgccaagctcctggtaaaggtttggagtgggtttcttctatccctccttctggtggccatacttcttatgctgactccgttaaaggtcgcttcactatctctagagacaactctaagaatactctctacttgcagatgaacagcttaagggctgaggacacggccgtgtattactgtgctaaaagcagtggctggtacgaggactactttgactactggggccagggaaccctggtcaccgtctcaagcggtggcggcggttcggggggtggcggaagtggaggtggagggagtcaagacatccagatgacccagtctccatcctccctgtctgcctctgtaggagacagagtcaccatcacttgccaggcgagtcaagatattaccaactatttaaattggtatctgcataaaccagggaaagcccctaagctcctgatctacgacgcatccaacttgcaaacaggggtcccatcaaggttcagtggaagtggatctgggacagattttactttcaccatcactagcctgcagcctgaagattttggaacatattactgtcaacagtatgatactttacatccctctttcggccctgggaccacagtggatatcaaaaccggtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagccggtggcagcaggggaacgtcttctcatgctccgtgatgcatgaggctctgcacaaccactacacgcagaagagcctctccctgtctccgggtaaatgagcggccgca

SEQ ID NO: 778 - DNA, 1515 bp, Artificial Sequence
  Made in a lab
aagcttgccgccatggattttcaagtgcagattttcagcttcctgctaatcagtgcttcagtcataatgtcgcgaggagaagttcaattgttagagtctggtggcggtcttgttcagcctggtggttctttacgtctttcttgcgctgcttccggattcactttctctgcttactctatggtttgggttcgccaagctcctggtaaaggtttggagtgggtttcttatatctatccttctggtggcattactacttatgctgactccgttaaaggtcgcttcactatctctagagacaactctaagaatactctctacttgcagatgaacagcttaagggctgaggacacggctgtgtattactgtgcgagagagggccaggtttttgatatctggggccaagggaccacggtcaccgtctcaagcggtggcggcggttcggggggtggcggaagtggaggtggagggagtcaagacatccagatgacccagtctccagccaccctgtctttgtctccaggggaaagagccaccctctcctgcagggccagtcagagtgttaccacctacttagcctggtaccaacagagacctggccaggctcccaggctcctcatctatgatgcatccaacagggccactggcatcccagccaggttcagtggcagtgggtctgggacagacttcactctcaccatcagcagcgtagagcctgaagattatgcagtttattactgtcagcagcgtagcaactggcctccttcgatcaccttcggccaagggacacgactggagagtaagaccggtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagccggtggcagcaggggaacgtcttctcatgctccgtgatgcatgaggctctgcacaaccactacacgcagaagagcctctccctgtctccgggtaaatgagcggccgca

SEQ ID NO: 779 - DNA, 1521 bp, Artificial Sequence
  Made in a lab
aagcttgccgccatggattttcaagtgcagattttcagcttcctgctaatcagtgcttcagtcataatgtcgcgaggagaagttcaattgttagagtctggtggcggtcttgttcagcctggtggttctttacgtctttcttgcgctgcttccggattcactttctctaattacgctatgggttgggttcgccaagctcctggtaaaggtttggagtgggtttctcgtatcgttccttctggtggcatgacttcttatgctgactccgttaaaggtcgcttcactatctctagagacaactctaagaatactctctacttgcagatgaacagcttaagggctgaggacacagccgtgtattactgtaccacagatgatttttggtccggtatggacgtctggggccaagggaccacggtcaccgtctcaagcggtggcggcggttcggggggtggcggaagtggaggtggagggagtcagagcgctttgactcagcctgcctccgtgtctgggtctcctggacagtcgatcaccatctcctgcactggaaccagcagtgatgttgggagttataaccttgtctcctggtaccaacagcacccaggcaaagcccccaaagtcatgatttatgagggcagtaagcggccctcaggggtttctaatcgcttctctggctccaagtctggcaacacggcctccctgacaatctctgggctccaggctgaggacgaggctgattattactgctgctcatatgcaggtcgtagcacttatgtcttcggaactgggaccaaggtcaccgtcctaaccggtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagccggtggcagcaggggaacgtcttctcatgctccgtgatgcatgaggctctgcacaaccactacacgcagaagagcctctccctgtctccgggtaaatgagcggccgca

SEQ ID NO: 780 - DNA, 1542 bp, Artificial Sequence
  Made in a lab
aagcttgccgccatggattttcaagtgcagattttcagcttcctgctaatcagtgcttcagtcataatgtcgcgaggagaagttcaattgttagagtctggtggcggtcttgttcagcctggtggttctttacgtctttcttgcgctgcttccggattcactttctctgcttacactatgcattgggttcgccaagctcctggtaaaggtttggagtgggtttcttctatctatccttctggtggcactactccttatgctgactccgttaaaggtcgcttcactatctctagagacaactctaagaatactctctacttgcagatgaacagcttaagggctgaggacacagccacatattactgtgccagagtggtgaatatcgatttttggagtggttacaatatgcggagtgcttttgatatctggggccaagggacaatggtcaccgtctcaagcggtggcggcggttcagggggtggcggaagtggaggtggagggagtcaagacatccagatgacccagtctccatcctccgtgtctgcgtctgtaggagacacagtcaccatcacttgtcgggcgagtcagggtgttagcgactggttagcctggtatcagcagaagccagggaaagcccctaacctcctaatctatggtgcatccagtctgcaaactggggtcccatcacggttcagcggcggtggatccgggacagatttcactctcaccatcagtagcctgcagcctgaggattttgcaacttactattgtcaacaggctcacagtttccctttcactttcggcggagggaccagggtggaaatcaaaaccggtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagccggtggcagcaggggaacgtcttctcatgctccgtgatgcatgaggctctgcacaaccactacacgcagaagagcctctccctgtctccgggtaaatgagcggccgca

SEQ ID NO: 781 - DNA, 1524 bp, Artificial Sequence
  Made in a lab
aagcttgccgccatggattttcaagtgcagattttcagcttcctgctaatcagtgcttcagtcataatgtcgcgaggagaagttcaattgttagagtctggtggcggtcttgttcagcctggtggttctttacgtctttcttgcgctgcttccggattcactttctctgagtacggtatgtcttgggttcgccaagctcctggtaaaggtttggagtgggtttcttctatccgttcttctggtggctggactaagtatgctgactccgttaaaggtcgcttcactatctctagagacaactctaagaatactctctacttgcagatgaacagcttaagggctgaggacacggccgtgtattactgtgcgaaaggcctaggggccactagtggggaatttgactattggggccagggaaccctggtcaccgtctcaagcggtggcggcggttcggggggtggcggaagtggaggtggagggagtcaagacatccagatgacccagtctccaggcaccctgtctttgtctccaggggaaagagccaccctctcctgcagggccagtcagagtgttagcggcaactacttagcctggtaccagcagaaacttggccaggctcccaggctcctcatctatggtgcatccagcagggccactggcaccccagacaggttcagtggcagtgggtctgggacagaattcactctcacaatcagcagcctgcagcctgaagattttgcaacttattactgtctacagcataatagttatccgctcactttcggcgaagggaccaaggtggaaatcaaaaccggtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagccggtggcagcaggggaacgtcttctcatgctccgtgatgcatgaggctctgcacaaccactacacgcagaagagcctctccctgtctccgggtaaatgagcggccgca

SEQ ID NO: 782 - DNA, 1529 bp, Artificial Sequence
  Made in a lab
aagcttgccgccatggattttcaagtgcagattttcagcttcctgctaatcagtgcttcagtcataatgtcgcgaggagaagttcaattgttagagtctggtggcggtcttgttcagcctggtggttctttacgtctttcttgcgctgcttccggattcactttctctcagtactttatgatgtgggttcgccaagctcctggtaaaggtttggagtgggtttcttctatctctccttctggtggctatactcagtatgctgactccgttaaaggtcgcttcactatctctagagacaactctaagaatactctctacttgcagatgaacagcttaagggctgaggacacggccgtgtattactgtgcgaaaaccggtgactacacacgctactactcctacggtatggacgtctggggccagggaaccctggtcaccgtctcaagcggtggcggcggttcggggggtggcggaagtggaggtggagggagtcaagacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcatcatcagcagtctgcaacctgaagattttgcaacttactactgtcaacagagttacagtaccccgctcactttcggcggagggaccaaggtggagatcaaaaccggtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagccggtggcagcaggggaacgtcttctcatgctccgtgatgcatgaggctctgcacaaccactacacgcagaagagcctctccctgtctccgggtaaatgagcggccgc

SEQ ID NO: 783 - DNA, 1533 bp, Artificial Sequence
  Made in a lab
aagcttgccgccatggattttcaagtgcagattttcagcttcctgctaatcagtgcttcagtcataatgtcgcgaggagaagttcaattgttagagtctggtggcggtcttgttcagcctggtggttctttacgtctttcttgcgctgcttccggattcactttctctggttactctatggcttgggttcgccaagctcctggtaaaggtttggagtgggtttctggtatctactcttctggtggctggacttggtatgctgactccgttaaaggtcgcttcactatctctagagacaactctaagaatactctctacttgcagatgaacagcttaagggctgaggacacagccgtgtattactgtacaagagaccaatctttctcctatgatagtagtcttgatgcttttgatatctggggccaagggacaatggtcaccgtctcaagcggtggcggcggttcggggggtggcggaagtggaggtggagggagtcaagacatccagatgacccagtctccatcctccctgtctgcatctgtaggagacagagtcaccatcacttgccgggcaagtcagagtattaacaactatttaaattggtatcagcagaaaccagggaaagcccctaagctcctgatctatactgcatccactttgcacagtggggtcccatcaaggttcagtggcagtggatctgggacagatttcactctcaccatcagcaatctgcaacctgaagattttgcaacttactactgtcaacagagttacgctaccctccggacgttcggccaagggaccaaggtggagatcaaaaccggtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagccggtggcagcaggggaacgtcttctcatgctccgtgatgcatgaggctctgcacaaccactacacgcagaagagcctctccctgtctccgggtaaatgagcggccgca

SEQ ID NO: 784 - DNA, 1539 bp, Artificial Sequence
  Made in a lab
aagcttgccgccatggattttcaagtgcagattttcagcttcctgctaatcagtgcttcagtcataatgtcgcgaggagaagttcaattgttagagtctggtggcggtcttgttcagcctggtggttctttacgtctttcttgcgctgcttccggattcactttctctctttacggtatgcattgggttcgccaagctcctggtaaaggtttggagtgggtttcttctatcgttccttctggtggccttactcgttatgctgactccgttaaaggtcgcttcactatctctagagacaactctaagaatactctctacttgcagatgaacagcttaagggctgaggacacagccacatattactgtgcgagactcgcgtattacgatttttggagtggtcgtgatgcttttgatatctggggccaagggacaatggtcaccgtctcaagcggtggcggcggttcggggggtggcggaagtggaggtggagggagtcagagcgtcttgactcagcctgcctccgtgtctgggtctcctggacagtcgatcaccatctcctgcagtggaagcagcagtgacgttggtcattatgactatgtctcctggtaccaacagcacccaggcaaggcccccaaactcatgatttatgatgtcagtaatcggccctcaggggtttctaatcgcttctctggctccaagtctggcaacacggcctccctgaccatctctgggctccaggctgacgacgaggctgagtattactgcagctcatatacaagcagcggcactcgagtcttcggaactgggaccaaggtcaccgtcctaaccggtgagcccaaatcttctgacaaaactcacacatgcccaccgtgcccagcacctgaagccgcgggtgcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggcgtacgcgtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagccggtggcagcaggggaacgtcttctcatgctccgtgatgcatgaggctctgcacaaccactacacgcagaagagcctctccctgtctccgggtaaatgagcggccgca

SEQ ID NO: 785 - protein, 23 aa, Artificial Sequence
  Made in lab
  VARIANT 23: Xaa = Any Amino Acid
MDFQVQIFSFLLISASVIMSRGX

SEQ ID NO: 786 - protein, 20 aa, Artificial Sequence
  Made in lab
MEAPAQLLFLLLLWLPDTTG

SEQ ID NO: 787 - protein, 15 aa, Artificial Sequence
  Made in a lab
RASESVHNFGISFMN

SEQ ID NO: 788 - protein, 7 aa, Artificial Sequence
  Made in a lab
TASNQGS

SEQ ID NO: 789 - protein, 9 aa, Artificial Sequence
  Made in a lab
HQGKEVPWT

SEQ ID NO: 790 - protein, 15 aa, Artificial Sequence
  Made in a lab
RASESVGNFGISFVN

SEQ ID NO: 791 - protein, 7 aa, Artificial Sequence
  Made in a lab
AASNQGS

SEQ ID NO: 792 - protein, 9 aa, Artificial Sequence
  Made in a lab
QQSKEVPYT

SEQ ID NO: 793 - protein, 6 aa, Artificial Sequence
  Made in a lab
NTYAMN

SEQ ID NO: 794 - protein, 19 aa, Artificial Sequence
  Made in a lab
RTVTKSNKYATYYADSVSD

SEQ ID NO: 795 - protein, 9 aa, Artificial Sequence
  Made in a lab
EDYYGTLDY

SEQ ID NO: 796 - protein, 5 aa, Artificial Sequence
  Made in a lab
SYWMH

SEQ ID NO: 797 - protein, 17 aa, Artificial Sequence
  Made in a lab
EINPSNGRTNYNERFKS

SEQ ID NO: 798 - protein, 9 aa, Artificial Sequence
  Made in a lab
SDTKATCDY

SEQ ID NO: 799 - protein, 111 aa, Artificial Sequence
  Made in a lab
EIVLTQSPPSLAVSLGQRATISCRASESVHNFGISFMNWFQQKSGQPPKLLIYTASNQGSGVPARFSGRGSGTDFSLIIHPVEEDDTAMYFCHQGKEVPWTFGGGTKLEIK

SEQ ID NO: 800 - protein, 111 aa, Artificial Sequence
  Made in a lab
DIVLTQSPASLAVSLGQRATISCRASESVGNFGISFVNWFQQKPGQPPKLLIYAASNQGSGVPARFSGSGSGTDFSLNIHPMEEDDTAMYFCQQSKEVPYTFGGGTKLEIK

SEQ ID NO: 801 - protein, 111 aa, Artificial Sequence
  Made in a lab
EIVLTQSPATLSLSPGERATLSCRASESVHNFGISFMNWYQQKPGQAPRLLIYTASNQGSGIPARFSGSGSGTDFTLTISSLEPEDFAVYYCHQGKEVPWTFGQGTKVEIK

SEQ ID NO: 802 - protein, 111 aa, Artificial Sequence
  Made in a lab
DIVLTQSPASLAVSPGQRATITCRASESVHNFGISFMNWYQQKPGQPPKLLIYTASNQGSGVPARFSGSGSGTDFTLTINPVEANDTANYYCHQGKEVPWTFGQGTKVEIK

SEQ ID NO: 803 - protein, 111 aa, Artificial Sequence
  Made in a lab
EIVLTQSPATLSLSPGERATLSCRASESVGNFGISFVNWYQQKPGQAPRLLIYAASNQGSGIPARFSGSGSGTDFTLTISSLEPEDFAVYYCQQSKEVPYTFGQGTKLEIK

SEQ ID NO: 804 - protein, 111 aa, Artificial Sequence
  Made in a lab
DIQMTQSPSSLSASVGDRVTITCRASESVGNFGISFVNWYQQKPGKAPKLLIYAASNQGSGVPSRFSGSGSGTDFTLTISSLQPEDFATYYCQQSKEVPYTFGQGTKLEIK

SEQ ID NO: 805 - protein, 120 aa, Artificial Sequence
  Made in a lab
EVQLVESGGGLVQPKGTLRLSCAASGFSFNTYAMNWVRQAPGKSLEWVARTVTKSNKYATYYADSVSDRVTISREDSQSMLYLQMTNLKTEDTAMYYCVREDYYGTLDYWGQGTSVTVSS

SEQ ID NO: 806 - protein, 118 aa, Artificial Sequence
  Made in a lab
QVQLQQPGAELVKPGASVKLSCKASGYIFTSYWMHWVKQRPGQGLEWIGEINPSNGRTNYNERFKSKATLTVDKSSSTAYIQLSSLTSDDSAVYYCSRSDTKATCDYWGQGTTLTVSS

SEQ ID NO: 807 - protein, 120 aa, Artificial Sequence
  Made in a lab
EVQLVESGGGLVQPGGSLRLSCAASGFTFNTYAMNWVRQAPGKGLEWVGRTVTKSNKYATYYADSVSDRFTISRDDSKNSLYLQMNSLKTEDTAVYYCAREDYYGTLDYWGQGTLVTVSS

SEQ ID NO: 808 - protein, 120 aa, Artificial Sequence; identical to SEQ ID NO: 807
  Made in a lab

SEQ ID NO: 809 - protein, 118 aa, Artificial Sequence
  Made in a lab
QVQLVQSGAEVKKPGASVKVSCKASGYIFTSYWMHWVRQAPGQGLEWMGEINPSNGRTNYNERFKSRVTMTRDTSISTAYMELSRLRSDDTAVYYCARSDTKATCDYWGQGTTVTVSS

SEQ ID NO: 810 - protein, 118 aa, Artificial Sequence; identical to SEQ ID NO: 809
  Made in a lab

SEQ ID NO: 811 - DNA, 333 bp, Artificial Sequence
  Made in a lab
gagattgtgctgacccaatctccaccttctttggctgtgtctctggggcagagggccaccatctcctgcagagccagcgaaagtgttcataattttggcattagttttatgaactggtttcaacagaaatcaggacagccacccaaactcctcatctatactgcatccaaccaaggatccggggtccctgccaggtttagtggcagagggtctgggacagacttcagtctcatcatccaccctgtggaggaagatgatactgcaatgtatttctgtcaccaaggtaaggaggttccgtggacgttcggtggaggcaccaagctagaaatcaaa

SEQ ID NO: 812 - DNA, 333 bp, Artificial Sequence
  Made in a lab
gacattgtgctgacccaatctccagcttctttggctgtgtctctagggcagagggccaccatctcctgcagagccagcgaaagtgttggtaattttggcattagttttgtgaactggttccaacagaaaccaggacagccacccaaactcctcatctatgctgcctccaaccaaggatccggggtccctgccaggtttagtggcagtgggtctgggacagacttcagcctcaacatccatcctatggaggaggatgatactgcaatgtatttctgtcagcaaagtaaggaggttccgtacacgttcggaggggggaccaagctggaaataaaa

SEQ ID NO: 813 - DNA, 333 bp, Artificial Sequence
  Made in a lab
gaaattgtgttgacacagtctccagccaccctgtctttgtctccaggggaaagagccaccctctcctgcagagccagcgaaagtgttcataattttggcattagttttatgaactggtaccaacagaaacctggccaggctcccaggctcctcatctatactgcatccaaccaaggatccggcatcccagccaggttcagtggcagtgggtctgggacagacttcactctcaccatcagcagcctagagcctgaagattttgcagtttattactgtcaccaaggtaaggaggttccgtggacgttcggccaagggaccaaggtggaaatcaaa

SEQ ID NO: 814 - DNA, 333 bp, Artificial Sequence
  Made in a lab
gacattgtgctgacccagtctccagcctccttggccgtgtctccaggacagagggccaccatcacctgcagagccagcgaaagtgttcataattttggcattagttttatgaactggtatcagcagaaaccaggacaacctcctaaactcctgatttacactgcatccaaccaaggatccggggtcccagccaggttcagcggcagtgggtctgggaccgatttcaccctcacaattaatcctgtggaagctaatgatactgcaaattattactgtcaccaaggtaaggaggttccgtggacgttcggccaagggaccaaggtggaaatcaaa

SEQ ID NO: 815 - DNA, 333 bp, Artificial Sequence
  Made in a lab
gaaattgtgttgacacagtctccagccaccctgtctttgtctccaggggaaagagccaccctctcctgcagagccagcgaaagtgttggtaattttggcattagttttgtgaactggtaccaacagaaacctggccaggctcccaggctcctcatctatgctgcctccaaccaaggatccggcatcccagccaggttcagtggcagtgggtctgggacagacttcactctcaccatcagcagcctagagcctgaagattttgcagtttattactgtcagcaaagtaaggaggttccgtacacttttggccaggggaccaagctggagatcaag

SEQ ID NO: 816 - DNA, 334 bp, Artificial Sequence
  Made in a lab
gacatccagatgacccagtctccatcctccctgtctgcatctgtaggagacagagtcaccatcacttgcagagccagcgaaagtgttggtaattttggcattagttttgtgaactggtatcagcagaaaccagggaaagcccctaagctcctgatctatgctgcctccaaccaaggatccggggtcccatcaaggttcagtggcagtggatctgggacagatttcactctcaccatcagcagtctgcaacctgaagattttgcaacttactactgtcagcaaagtaaggaggttccgtacacttttggccaggggaccaagctggagatcaagt

SEQ ID NO: 817 - DNA, 360 bp, Artificial Sequence
  Made in a lab
gaggtgcagcttgttgagtctggtggaggattggtacagcctaaagggacattgagactctcatgtgccgcctctggattcagcttcaatacctatgccatgaactgggtccgccaggctccaggaaagagtttggagtgggttgctcgcacagtgactaaaagtaataagtatgcaacatattatgcagattcagtgagtgacagagtcaccatctccagagaggattcacaaagcatgctttatctgcaaatgaccaacttgaaaactgaggacacagccatgtattactgtgtgagagaagattactacggcactctggactactggggtcaaggaacctcagtcaccgtctcctca

SEQ ID NO: 818 - DNA, 354 bp, Artificial Sequence
  Made in a lab
caggtccaactgcagcagcctggggctgaactggtgaagcctggggcttctgtgaagctgtcctgcaaggcttctggctacatcttcaccagctactggatgcactgggtgaagcagaggcctggacaaggccttgagtggattggagagattaatcctagcaacggtcgtactaactacaatgagaggttcaagagcaaggccacactgactgtagacaaatcctccagcacagcctacattcaactcagcagcctgacatctgatgactctgcggtctattactgttcaagatcggatactaaggctacgtgtgactactggggccaaggcaccactctcacagtctcctca

SEQ ID NO: 819 - DNA, 360 bp, Artificial Sequence
  Made in a lab
gaggtgcagctggtggagtctgggggaggcttggtccagcctggagggtccctgagactctcctgtgcagcctctggattcaccttcaatacctatgccatgaactgggtccgccaggctccagggaaggggctggagtgggttggccgcacagtgactaaaagtaataagtatgcaacatattatgcagattcagtgagtgacagattcaccatctcaagagatgattcaaagaactcactgtatctgcaaatgaacagcctgaaaaccgaggacacggccgtgtattactgtgctagagaagattactacggcactctggactactggggccaaggaaccctggtcaccgtctcctca

SEQ ID NO: 820 - DNA, 360 bp, Artificial Sequence; identical to SEQ ID NO: 819
  Made in a lab

SEQ ID NO: 821 - DNA, 354 bp, Artificial Sequence
  Made in a lab
caggtgcagctggtgcagtctggggctgaggtgaagaagcctggggcctcagtgaaggtctcctgcaaggcttctggctacatcttcaccagctactggatgcactgggtgcgacaggcccctggacaagggcttgagtggatgggagagattaatcctagcaacggtcgtactaactacaatgagaggttcaagagcagggtcaccatgaccagggacacgtccatcagcacagcctacatggagctgagcaggctgagatctgacgacacggccgtgtattactgtgcgagatcggatactaaggctacgtgtgactactggggccaagggaccacggtcaccgtctcctct

SEQ ID NO: 822 - DNA, 354 bp, Artificial Sequence; identical to SEQ ID NO: 821
  Made in a lab

SEQ ID NO: 823 - protein, 21 aa, Artificial Sequence
  Made in a lab
QRHNNSSLNTGTQMAGHSPNS

SEQ ID NO: 824 - protein, 21 aa, Artificial Sequence
  Made in a lab
QRHNNSSLNTGTQKARHSPNS

SEQ ID NO: 825 - protein, 21 aa, Artificial Sequence
  Made in a lab
QRHNNSSLNTGTQMARHSPNS

SEQ ID NO: 826 - protein, 21 aa, Artificial Sequence
  Made in a lab
QRHNNSSLNTRTQKAGHSPNS

SEQ ID NO: 827 - protein, 21 aa, Artificial Sequence
  Made in a lab
QRHNNSSLNTRTQMAGHSPNS

SEQ ID NO: 828 - protein, 21 aa, Artificial Sequence
  Made in a lab
QRHNNSSLNTRTQMARHSPNS

SEQ ID NO: 829 - skipped

SEQ ID NO: 830 - protein, 10 aa, Human
EPKSCDKTHT

SEQ ID NO: 831 - protein, 7 aa, Human
ERKCCVE

SEQ ID NO: 832 - protein, 12 aa, Human
ELKTPLGDTTHT

SEQ ID NO: 833 - protein, 10 aa, Human
EPKSCDTPPP

SEQ ID NO: 834 - protein, 7 aa, Human
ESKYGPP

SEQ ID NO: 835 - protein, 5 aa, Human
CPPCP

SEQ ID NO: 836 - protein, 5 aa, Human
CPRCP

SEQ ID NO: 837 - protein, 5 aa, Human
CPSCP

SEQ ID NO: 838 - protein, 17 aa, Human
ELKTPLGDTTHTCPRCP

SEQ ID NO: 839 - protein, 15 aa, Human
EPKSCDTPPPCPRCP

SEQ ID NO: 840 - protein, 34 aa, Human
ESPKAQASSVPTAQPQAEGSLAKATTAPATTRNT

SEQ ID NO: 841 - protein, 24 aa, Human
GRGGEEKKKEKEKEEQEERETKTP

SEQ ID NO: 842 - protein, 19 aa, Human
VPSTPPTPSPSTPPTPSPS

SEQ ID NO: 843 - protein, 6 aa, Human
VPPPPP

SEQ ID NO: 844 - protein, 107 aa, Human
VCSRDFTPPTVKILQSSSDGGGHFPPTIQLLCLVSGYTPGTINITWLEDGQVMDVDLSTASTTQEGELASTQSELTLSQKHWLSDRTYTCQVTYQGHTFEDSTKKCA

SEQ ID NO: 845 - protein, 112 aa, Human
VIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVP

SEQ ID NO: 846 - protein, 257 aa, Homo sapiens; identical to SEQ ID NO: 695

SEQ ID NO: 847 - protein, 211 aa, Homo sapiens; identical to SEQ ID NO: 696

SEQ ID NO: 848 - protein, 22 aa, Artificial Sequence
  Made in lab
MDFQVQIFSFLLISASVIMSRG

SEQ ID NO: 849 - protein, 24 aa, Artificial Sequence
  Made in lab
  VARIANT 23, 24: Xaa = Any Amino Acid
MDFQVQIFSFLLISASVIMSRGXX

SEQ ID NO: 850 - protein, 25 aa, Artificial Sequence
  Made in lab
  VARIANT 23, 24, 25: Xaa = Any Amino Acid
MDFQVQIFSFLLISASVIMSRGXXX

SEQ ID NO: 851 - protein, 19 aa, Homo Sapiens
VPSTPPTPSPSTPPTPSPS

SEQ ID NO: 852 - protein, 6 aa, Homo sapiens
VPPPPP

SEQ ID NO: 853 - protein, 58 aa, Homo sapiens
ESPKAQASSVPTAQPQAEGSLAKATTAPATTRNTGRGGEEKKKEKEKEEQEERETKTP

SEQ ID NO: 854 - protein, 15 aa, Homo sapiens
EPKSCDKTHTCPPCP

SEQ ID NO: 855 - protein, 12 aa, Homo sapiens
ERKCCVECPPCP

SEQ ID NO: 856 - protein, 62 aa, Homo sapiens
ELKTPLGDTTHTCPRCPEPKSCDTPPPCPRCPEPKSCDTPPPCPRCPEPKSCDTPPPCPRCP

SEQ ID NO: 857 - protein, 12 aa, Homo sapiens
ESKYGPPCPSCP

SEQ ID NO: 858 - protein, 20 aa, Artificial Sequence
  NKG2D Linker
NSLFNQEVQIPLTESYSPNS

SEQ ID NO: 859 - protein, 12 aa, Artificial Sequence
  Stalk  region of CD69
QYNCPGQYTFSM

SEQ ID NO: 860 - protein, 11 aa, Artificial Sequence
  Stalk  region of CD72
PFFTCGSADTC

SEQ ID NO: 861 - protein, 18 aa, Artificial Sequence
  Stalk  region of CD94
EPAFTPGPNIELQKDSDC

SEQ ID NO: 862 - protein, 18 aa, Artificial Sequence
  Stalk  region of NKG2A
QRHNNSSLNTRTQKARHC

SEQ ID NO: 863 - protein, 17 aa, Artificial Sequence
  NKG2D Linker
NSLFNQEVQIPLTESYS

SEQ ID NO: 864 - protein, 20 aa, Artificial Sequence
  Made in a lab
NSLFNQEVQIPLTESYCPNS

SEQ ID NO: 865 - protein, 15 aa, Artificial Sequence
  Altered human IgG1 hinge (SCC-P)
EPKSSDKTHTCPPCP

SEQ ID NO: 866 - protein, 15 aa, Artificial Sequence
  Altered human IgG1 hinge (SSC-P)
EPKSSDKTHTSPPCP

SEQ ID NO: 867 - protein, 15 aa, Artificial Sequence
  Altered human IgG1 hinge (SSS-P)
EPKSSDKTHTSPPSP

SEQ ID NO: 868 - protein, 15 aa, Artificial Sequence
  Altered human IgG1 hinge (CSC-P)
EPKSCDKTHTSPPCP

SEQ ID NO: 869 - protein, 15 aa, Artificial Sequence
  Altered human IgG1 hinge (CSS-P)
EPKSCDKTHTSPPSP

SEQ ID NO: 870 - protein, 15 aa, Artificial Sequence
  Altered human IgG1 hinge (CCS-P)
EPKSCDKTHTCPPSP

SEQ ID NO: 871 - protein, 15 aa, Artificial Sequence
  Altered human IgG1 hinge (SCC-S)
EPKSSDKTHTCPPCS

SEQ ID NO: 872 - protein, 15 aa, Artificial Sequence
  Altered human IgG1 hinge (SSC-S)
EPKSSDKTHTSPPCS

SEQ ID NO: 873 - protein, 15 aa, Artificial Sequence
  Altered human IgG1 hinge (SSS-S)
EPKSSDKTHTSPPSS

SEQ ID NO: 874 - protein, 15 aa, Artificial Sequence
  Altered human IgG1 hinge (CSC-S)
EPKSCDKTHTSPPCS

SEQ ID NO: 875 - protein, 15 aa, Artificial Sequence
  Altered human IgG1 hinge (CSS-S)
EPKSCDKTHTSPPSS

SEQ ID NO: 876 - protein, 15 aa, Artificial Sequence
  Altered human IgG1 hinge (CCS-S)
EPKSCDKTHTCPPSS

SEQ ID NO: 877 - protein, 107 aa, Artificial Sequence
  Altered human CH2 upper hinge like sequence of IgE
VCSRDFTPPTVKILQSSSDGGGHFPPTIQLLCLVSGYTPGTINITWLEDGQVMDVDLSTASTTQEGELASTQSELTLSQKHWLSDRTYTCQVTYQGHTFEDSTKKSA

SEQ ID NO: 878 - protein, 15 aa, Artificial Sequence
  Made in a lab
EPKSSDKTHTCPPCP

SEQ ID NO: 879 - protein, 15 aa, Artificial Sequence
  Made in a lab
EPKSSDKTHTCPPCS

SEQ ID NO: 880 - protein, 15 aa, Artificial Sequence
  Made in a lab
EPKSCDKTHTSPPCS

SEQ ID NO: 881 - protein, 15 aa, Artificial Sequence
  Made in a lab
EPKSCDKTHTCPPCP

SEQ ID NO: 882 - protein, 15 aa, Artificial Sequence
  Made in a lab
EPKSCDKTHTSPPSS

SEQ ID NO: 883 - protein, 15 aa, Artificial Sequence
  Made in a lab
PRGPTIKPCPPCKCP

SEQ ID NO: 884 - protein, 15 aa, Artificial Sequence
  Made in a lab
EPKSSDKTHTSPPSS

SEQ ID NO: 885 - protein, 15 aa, Artificial Sequence
  Made in a lab
EPKSSDKTHTSPPCS

SEQ ID NO: 886 - protein, 15 aa, Artificial Sequence
  Made in a lab
EPKSSDKTHTCPPSS

SEQ ID NO: 887 - protein, 15 aa, Artificial Sequence
  Made in a lab
EPKSCDKTHTSPPCP

SEQ ID NO: 888 - protein, 15 aa, Artificial Sequence
  Made in a lab
EPKSSDKTHTSPPSP

SEQ ID NO: 889 - protein, 15 aa, Artificial Sequence
  Made in a lab
EPKSSDKTHTSPPCP

SEQ ID NO: 890 - protein, 15 aa, Artificial Sequence
  Made in a lab
EPKSCDKTHTSPPSP

SEQ ID NO: 891 - protein, 15 aa, Artificial Sequence
  Made in a lab
EPKSSDKTHTCPPSP

SEQ ID NO: 892 - protein, 10 aa, Artificial Sequence
  Hinge sequence
PPPPPCPPCP

SEQ ID NO: 893 - protein, 16 aa, Artificial Sequence
  Hinge sequence
TEPKSSDKTHTCPPCP

SEQ ID NO: 894 - protein, 20 aa, Artificial Sequence
  Hinge sequence
EPKSSDKTHTSPPSPCPPCP

SEQ ID NO: 895 - protein, 20 aa, Artificial Sequence
  Hinge sequence
EPKSSDTPPPSPRSPCPPCP

SEQ ID NO: 896 - protein, 23 aa, Artificial Sequence
  Hinge sequence
PSTPPTPSPSTPPTPSPSCPPCP

SEQ ID NO: 897 - protein, 23 aa, Artificial Sequence
  Hinge sequence
RTEPRVPITQNPCPPLKECPPCA

SEQ ID NO: 898 - protein, 39 aa, Artificial Sequence
  Hinge sequence
ESPKAQASSVPTAQPQAEGSLAKATTAPATTRNTCPPCP

SEQ ID NO: 899 - protein, 50 aa, Artificial Sequence
  Hinge sequence
QEKEAIERLKAAGAPESLVIQAYFASEKNENLAANFLLSQNFDDECPPCP

SEQ ID NO: 900 - protein, 52 aa, Artificial Sequence
  Hinge sequence
QEKEAIERLKAAGAPESLVIQAYFASEKNENLAANFLLSQNFDDECPPCPSG

SEQ ID NO: 901 - protein, 110 aa, Artificial Sequence
  Hinge sequence
SRDFTPPTVKILQSSSDGGGHFPPTIQLLCLVSGYTPGTINITWLEDGQVMDVDLSTASTTQEGELASTQSELTLSQKHWLSDRTYTCQVTYQGHTFEDSTKKSACPPCP

SEQ ID NO: 902 - protein, 111 aa, Artificial Sequence
  Hinge sequence
SRDFTPPTVKILQSSSDGGGHFPPTIQLLCLVSGYTPGTINITWLEDGQVMDVDLSTASTTQEGELASTQSELTLSQKHWLSDRTYTCQVTYQGHTFEDSTKKSACPPCSG

SEQ ID NO: 903 - protein, 116 aa, Artificial Sequence
  Hinge sequence
IAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMSVPCPPCP

SEQ ID NO: 904 - protein, 7 aa, Artificial Sequence
  Peptide linker
SPPSPNS

SEQ ID NO: 905 - protein, 17 aa, Artificial Sequence
  Peptide linker
EPTSTDKTHTSPPSPNS

SEQ ID NO: 906 - protein, 17 aa, Artificial Sequence
  Peptide linker
EPTSTDKTHTCPPCPNS

SEQ ID NO: 907 - protein, 16 aa, Artificial Sequence
  Peptide linker
NQEVQIPLTESYSPNS

SEQ ID NO: 908 - protein, 20 aa, Artificial Sequence
  Peptide linker
NSGANQEVQIPLTESYSPNS

SEQ ID NO: 909 - protein, 14 aa, Artificial Sequence
  Peptide linker
EVQIPLTESYSPNS

SEQ ID NO: 910 - protein, 20 aa, Artificial Sequence
  Peptide linker
NSGFNQEVQIPLTESYSPNS

SEQ ID NO: 911 - protein, 20 aa, Artificial Sequence
  Peptide linker
NSLANQEVQIPLTESYSPNS

SEQ ID NO: 912 - protein, 20 aa, Artificial Sequence
  Peptide linker
NSLFSQEVQIPLTESYSPNS

SEQ ID NO: 913 - protein, 20 aa, Artificial Sequence
  Peptide linker
NSLASQEVQIPLTESYSPNS

SEQ ID NO: 914 - protein, 26 aa, Artificial Sequence
  Peptide linker
EQKLSNMENRLKPFFTSGSADTSPNS

SEQ ID NO: 915 - protein, 21 aa, Artificial Sequence
  Peptide linker
QRHANSALNTGTQMAGHSPNS

SEQ ID NO: 916 - protein, 21 aa, Artificial Sequence
  Peptide linker
QRHNAASLNTGTQMAGHSPNS

SEQ ID NO: 917 - protein, 21 aa, Artificial Sequence
  Peptide linker
QRHAASSLNTGTQMAGHSPNS

SEQ ID NO: 918 - protein, 19 aa, Artificial Sequence
  Peptide linker
QRHSSLNTGTQMAGHSPNS

SEQ ID NO: 919 - protein, 21 aa, Artificial Sequence
  Peptide linker
QRHGGSSLNTGTQMAGHSPNS

SEQ ID NO: 920 - protein, 11 aa, Artificial Sequence
  Peptide linker
HDGSSLNTPNS

SEQ ID NO: 921 - protein, 16 aa, Artificial Sequence
  Peptide linker
SSLNTGTQMAGHSPNS

SEQ ID NO: 922 - protein, 16 aa, Artificial Sequence
  Peptide linker
SSLNTRTQMAGHSPNS

SEQ ID NO: 923 - protein, 16 aa, Artificial Sequence
  Peptide linker
SSLNTGTQMARHSPNS

SEQ ID NO: 924 - protein, 16 aa, Artificial Sequence
  Peptide linker
SSLNTGTQKAGHSPNS

SEQ ID NO: 925 - protein, 16 aa, Artificial Sequence
  Peptide linker
SSLNTGTQKARHSPNS

SEQ ID NO: 926 - protein, 16 aa, Artificial Sequence
  Peptide linker
SSLNTRTQMARHSPNS

SEQ ID NO: 927 - protein, 16 aa, Artificial Sequence
  Peptide linker
SSLNTRTQKAGHSPNS

SEQ ID NO: 928 - protein, 17 aa, Artificial Sequence
  Peptide linker
NSLFNQEVQIPLTEPNS

SEQ ID NO: 929 - protein, 14 aa, Artificial Sequence
  Peptide linker
NSLFNQEVQIPPNS

SEQ ID NO: 930 - protein, 11 aa, Artificial Sequence
  Peptide linker
NSLFNQEVPNS

SEQ ID NO: 931 - protein, 13 aa, Artificial Sequence
  Peptide linker
QRHNNSSLNTPNS

SEQ ID NO: 932 - protein, 19 aa, Artificial Sequence
  Peptide linker
SGGGGSGGGGSGGGGSPNS

SEQ ID NO: 933 - protein, 20 aa, Artificial Sequence
  H68 linker (scorpion linker derived from NKG2D)
NSLFNQEVQIPLTESYSPNS

SEQ ID NO: 934 - protein, 15 aa, Artificial Sequence
  Linker 134
EPMSCDKTHTCPPCP

SEQ ID NO: 935 - protein, 15 aa, Artificial Sequence
  Linker 135
EPMSSDKTHTCPPCP

SEQ ID NO: 936 - protein, 15 aa, Artificial Sequence
  Linker 136
EPMSCDKTHTSPPCP

SEQ ID NO: 937 - protein, 15 aa, Artificial Sequence
  Linker 137
EPMSCDKTHTCPPSP

SEQ ID NO: 938 - protein, 15 aa, Artificial Sequence
  Linker 138
EPMSSDKTHTSPPCP

SEQ ID NO: 939 - protein, 15 aa, Artificial Sequence
  Linker 139
EPMSSDKTHTCPPSP

SEQ ID NO: 940 - protein, 15 aa, Artificial Sequence
  Linker 140
EPMSCDKTHTSPPSP

SEQ ID NO: 941 - protein, 15 aa, Artificial Sequence
  Linker 141
EPMSCDKTHTCPPCS

SEQ ID NO: 942 - protein, 15 aa, Artificial Sequence
  Linker 142
EPMSSDKTHTCPPCS

SEQ ID NO: 943 - protein, 15 aa, Artificial Sequence
  Linker 143
EPMSCDKTHTSPPCS

SEQ ID NO: 944 - protein, 15 aa, Artificial Sequence
  Linker 144
EPMSCDKTHTCPPSS

SEQ ID NO: 945 - protein, 15 aa, Artificial Sequence
  Linker 145
EPMSSDKTHTSPPCS

SEQ ID NO: 946 - protein, 15 aa, Artificial Sequence
  Linker 146
EPMSSDKTHTCPPSP

SEQ ID NO: 947 - protein, 15 aa, Artificial Sequence
  Linker 147
EPMSCDKTHTSPPSS

SEQ ID NO: 948 - protein, 15 aa, Artificial Sequence
  Linker 148
EPTSCDKTHTCPPCP

SEQ ID NO: 949 - protein, 15 aa, Artificial Sequence
  Linker 149
EPTSSDKTHTCPPCP

SEQ ID NO: 950 - protein, 15 aa, Artificial Sequence
  Linker 150
EPTSCDKTHTSPPCP

SEQ ID NO: 951 - protein, 15 aa, Artificial Sequence
  Linker 151
EPTSCDKTHTCPPSP

SEQ ID NO: 952 - protein, 15 aa, Artificial Sequence
  Linker 152
EPTSSDKTHTSPPCP

SEQ ID NO: 953 - protein, 15 aa, Artificial Sequence
  Linker 153
EPTSSDKTHTCPPSP

SEQ ID NO: 954 - protein, 15 aa, Artificial Sequence
  Linker 154
EPTSCDKTHTSPPSP

SEQ ID NO: 955 - protein, 15 aa, Artificial Sequence
  Linker 155
EPTSCDKTHTCPPCS

SEQ ID NO: 956 - protein, 15 aa, Artificial Sequence
  Linker 156
EPTSSDKTHTCPPCS

SEQ ID NO: 957 - protein, 15 aa, Artificial Sequence
  Linker 157
EPTSCDKTHTSPPCS

SEQ ID NO: 958 - protein, 15 aa, Artificial Sequence
  Linker 158
EPTSCDKTHTCPPSS

SEQ ID NO: 959 - protein, 15 aa, Artificial Sequence
  Linker 159
EPTSSDKTHTSPPCS

SEQ ID NO: 960 - protein, 15 aa, Artificial Sequence
  Linker 160
EPTSSDKTHTCPPSS

SEQ ID NO: 961 - protein, 15 aa, Artificial Sequence
  Linker 161
EPTSCDKTHTSPPSS

SEQ ID NO: 962 - protein, 15 aa, Artificial Sequence
  Linker 162
EPASCDKTHTCPPCP

SEQ ID NO: 963 - protein, 15 aa, Artificial Sequence
  Linker 163
EPASSDKTHTCPPCP

SEQ ID NO: 964 - protein, 15 aa, Artificial Sequence
  Linker 164
EPASCDKTHTSPPCP

SEQ ID NO: 965 - protein, 15 aa, Artificial Sequence
  Linker 165
EPASCDKTHTCPPSP

SEQ ID NO: 966 - protein, 15 aa, Artificial Sequence
  Linker 166
EPASSDKTHTSPPCP

SEQ ID NO: 967 - protein, 15 aa, Artificial Sequence
  Linker 167
EPASSDKTHTCPPSP

SEQ ID NO: 968 - protein, 15 aa, Artificial Sequence
  Linker 168
EPASCDKTHTSPPSP

SEQ ID NO: 969 - protein, 15 aa, Artificial Sequence
  Linker 169
EPASCDKTHTCPPCS

SEQ ID NO: 970 - protein, 15 aa, Artificial Sequence
  Linker 170
EPASSDKTHTCPPCS

SEQ ID NO: 971 - protein, 15 aa, Artificial Sequence
  Linker 171
EPASCDKTHTSPPCS

SEQ ID NO: 972 - protein, 15 aa, Artificial Sequence
  Linker 172
EPASCDKTHTCPPSS

SEQ ID NO: 973 - protein, 15 aa, Artificial Sequence
  Linker 173
EPASSDKTHTSPPCS

SEQ ID NO: 974 - protein, 15 aa, Artificial Sequence
  Linker 174
EPASSDKTHTCPPSS

SEQ ID NO: 975 - protein, 15 aa, Artificial Sequence
  Linker 175
EPASCDKTHTSPPSS

SEQ ID NO: 976 - protein, 14 aa, Artificial Sequence
  Linker 176
EPSCDKTHTCPPCP

SEQ ID NO: 977 - protein, 14 aa, Artificial Sequence
  Linker 177
EPSSDKTHTCPPCP

SEQ ID NO: 978 - protein, 14 aa, Artificial Sequence
  Linker 178
EPSCDKTHTSPPCP

SEQ ID NO: 979 - protein, 14 aa, Artificial Sequence
  Linker 179
EPSCDKTHTCPPSP

SEQ ID NO: 980 - protein, 14 aa, Artificial Sequence
  Linker 180
EPSSDKTHTSPPCP

SEQ ID NO: 981 - protein, 14 aa, Artificial Sequence
  Linker 181
EPSSDKTHTCPPSP

SEQ ID NO: 982 - protein, 14 aa, Artificial Sequence
  Linker 182
EPSCDKTHTSPPSP

SEQ ID NO: 983 - protein, 14 aa, Artificial Sequence
  Linker 183
EPSCDKTHTCPPCS

SEQ ID NO: 984 - protein, 14 aa, Artificial Sequence
  Linker 184
EPSSDKTHTCPPCS

SEQ ID NO: 985 - protein, 14 aa, Artificial Sequence
  Linker 185
EPSCDKTHTSPPCS

SEQ ID NO: 986 - protein, 14 aa, Artificial Sequence
  Linker 186
EPSCDKTHTCPPSS

SEQ ID NO: 987 - protein, 14 aa, Artificial Sequence
  Linker 187
EPSSDKTHTSPPCS

SEQ ID NO: 988 - protein, 14 aa, Artificial Sequence
  Linker 188
EPSSDKTHTCPPSS

SEQ ID NO: 989 - protein, 14 aa, Artificial Sequence
  Linker 189
EPSCDKTHTSPPSS

SEQ ID NO: 990 - protein, 22 aa, Artificial Sequence
  Made in a lab
RTQRHNNSSLNTRTQKARHSGH

SEQ ID NO: 991 - protein, 118 aa, Artificial Sequence
  Made in a lab
RTRYLQVSQQLQQTNRVLEVTNSSLRQQLRLKITQLGQSAEDLQGSRRELAQSQEALQVEQRAHQAAEGQLQACQADRQKTKETLQSEEQQRRALEQKLSNMENRLKPFFTCGSADTC

SEQ ID NO: 992 - protein, 35 aa, Artificial Sequence
  Made in a lab
RTQSEEQQRRALEQKLSNMENRLKPFFTCGSADTC

SEQ ID NO: 993 - protein, 25 aa, Artificial Sequence
  Made in a lab
RTEQKLSNMENRLKPFFTCGSADTC

SEQ ID NO: 994 - protein, 21 aa, Artificial Sequence
  Made in a lab
QRHNNSSLNTGTQMAGHSPNS

SEQ ID NO: 995 - protein, 21 aa, Artificial Sequence
  Made in a lab
QRHNNSSLNTGTQKARHSPNS

SEQ ID NO: 996 - protein, 21 aa, Artificial Sequence
  Made in a lab
QRHNNSSLNTGTQMARHSPNS

SEQ ID NO: 997 - protein, 21 aa, Artificial Sequence
  Made in a lab
QRHNNSSLNTRTQKAGHSPNS

SEQ ID NO: 998 - protein, 21 aa, Artificial Sequence
  Made in a lab
QRHNNSSLNTRTQMAGHSPNS

SEQ ID NO: 999 - protein, 21 aa, Artificial Sequence
  Made in a lab
QRHNNSSLNTRTQMARHSPNS

SEQ ID NO: 1000 - protein, 2 aa, Artificial Sequence
  Made in a lab
NS

SEQ ID NO: 1001 - protein, 6 aa, Artificial Sequence
  Made in a lab
SCPPCP

SEQ ID NO: 1002 - protein, 8 aa, Artificial Sequence
  Made in a lab
GGGGSGNS

SEQ ID NO: 1003 - protein, 8 aa, Artificial Sequence
  Made in a lab
GCPPCPNS

SEQ ID NO: 1004 - protein, 8 aa, Artificial Sequence
  Made in a lab
GSPPSPNS

SEQ ID NO: 1005 - protein, 8 aa, Artificial Sequence
  Made in a lab
GSPPSPNS

SEQ ID NO: 1006 - protein, 8 aa, Artificial Sequence
  Made in a lab
GCPPCPNS

SEQ ID NO: 1007 - protein, 8 aa, Artificial Sequence
  Made in a lab
GCPPCPNS

SEQ ID NO: 1008 - protein, 9 aa, Artificial Sequence
  Made in a lab
GCPPCPGNS

SEQ ID NO: 1009 - protein, 9 aa, Artificial Sequence
  Made in a lab
GCPPCPANS

SEQ ID NO: 1010 - protein, 9 aa, Artificial Sequence
  Made in a lab
GCPPCPANS

SEQ ID NO: 1011 - protein, 9 aa, Artificial Sequence
  Made in a lab
EEEEDEGNS

SEQ ID NO: 1012 - protein, 10 aa, Artificial Sequence
  Made in a lab
NYGGGGSGNS

SEQ ID NO: 1013 - protein, 10 aa, Artificial Sequence
  Made in a lab
VSERPFPPNS

SEQ ID NO: 1014 - protein, 11 aa, Artificial Sequence
  Made in a lab
EPKSCDKTCCP

SEQ ID NO: 1015 - protein, 11 aa, Artificial Sequence
  Made in a lab
SQPEIVPISNS

SEQ ID NO: 1016 - protein, 12 aa, Artificial Sequence
  Made in a lab
GGGGSCPPCPNS

SEQ ID NO: 1017 - protein, 12 aa, Artificial Sequence
  Made in a lab
KADFLTPSIGNS

SEQ ID NO: 1018 - protein, 12 aa, Artificial Sequence
  Made in a lab
QMNSELSVLANS

SEQ ID NO: 1019 - protein, 13 aa, Artificial Sequence
  Made in a lab
EPKSCDKTHTCCP

SEQ ID NO: 1020 - protein, 13 aa, Artificial Sequence
  Made in a lab
EPKSCDKTCPPCP

SEQ ID NO: 1021 - protein, 13 aa, Artificial Sequence
  Made in a lab
GGGGSGGGGSGNS

SEQ ID NO: 1022 - protein, 13 aa, Artificial Sequence
  Made in a lab
GCPPCPGGGGSNS

SEQ ID NO: 1023 - protein, 13 aa, Artificial Sequence
  Made in a lab
GGGGSCPPCPGNS

SEQ ID NO: 1024 - protein, 13 aa, Artificial Sequence
  Made in a lab
GCPPCPGGGGSNS

SEQ ID NO: 1025 - protein, 13 aa, Artificial Sequence
  Made in a lab
GGGASCPPCPGNS

SEQ ID NO: 1026 - protein, 13 aa, Artificial Sequence
  Made in a lab
GGGASCPPCAGNS

SEQ ID NO: 1027 - protein, 13 aa, Artificial Sequence
  Made in a lab
GGGASCPPCAGNS

SEQ ID NO: 1028 - protein, 15 aa, Artificial Sequence
  Made in a lab
NYGGGGSGGGGSGNS

SEQ ID NO: 1029 - protein, 15 aa, Artificial Sequence
  Made in a lab
LSVKADFLTPSIGNS

SEQ ID NO: 1030 - protein, 15 aa, Artificial Sequence
  Made in a lab
LSVLANFSQPEIGNS

SEQ ID NO: 1031 - protein, 15 aa, Artificial Sequence
  Made in a lab
LKIQERVSKPKISNS

SEQ ID NO: 1032 - protein, 15 aa, Artificial Sequence
  Made in a lab
LDVSERPFPPHIQNS

SEQ ID NO: 1033 - protein, 15 aa, Artificial Sequence
  Made in a lab
REQLAEVTLSLKANS

SEQ ID NO: 1034 - protein, 15 aa, Artificial Sequence
  Made in a lab
RIHQMNSELSVLANS

SEQ ID NO: 1035 - protein, 15 aa, Artificial Sequence
  Made in a lab
DTKGKNVLEKIFSNS

SEQ ID NO: 1036 - protein, 15 aa, Artificial Sequence
  Made in a lab
LPPETQESQEVTLNS

SEQ ID NO: 1037 - protein, 15 aa, Artificial Sequence
  Made in a lab
RIHLNVSERPFPPNS

SEQ ID NO: 1038 - protein, 15 aa, Artificial Sequence
  Made in a lab
LSVKADFLTPSIGNS

SEQ ID NO: 1039 - protein, 15 aa, Artificial Sequence
  Made in a lab
LSVLANFSQPEIGNS

SEQ ID NO: 1040 - protein, 15 aa, Artificial Sequence
  Made in a lab
LSVLANFSQPEIGNS

SEQ ID NO: 1041 - protein, 15 aa, Artificial Sequence
  Made in a lab
RIHQMNSELSVLANS

SEQ ID NO: 1042 - protein, 15 aa, Artificial Sequence
  Made in a lab
KPFFTCGSADTCPNS

SEQ ID NO: 1043 - protein, 15 aa, Artificial Sequence
  Made in a lab
KPFFTCGSADTCPNS

SEQ ID NO: 1044 - protein, 15 aa, Artificial Sequence
  Made in a lab
QYNCPGQYTFSMPNS

SEQ ID NO: 1045 - protein, 15 aa, Artificial Sequence
  Made in a lab
GGGGSGGGGSGGGGS

SEQ ID NO: 1046 - protein, 15 aa, Artificial Sequence
  Made in a lab
GGGGCDKTHTCPPCP

SEQ ID NO: 1047 - protein, 15 aa, Artificial Sequence
  Made in a lab
EPKSCGGGGGCPPCP

SEQ ID NO: 1048 - protein, 15 aa, Artificial Sequence
  Made in a lab
EPKSCDKTHTCGGCP

SEQ ID NO: 1049 - protein, 15 aa, Artificial Sequence
  Made in a lab
EPKSCDKTHTCPPCG

SEQ ID NO: 1050 - protein, 15 aa, Artificial Sequence
  Made in a lab
EPKSCDKTHTCPPSP

SEQ ID NO: 1051 - protein, 15 aa, Artificial Sequence
  Made in a lab
EPKSCDKCHTCPPCP

SEQ ID NO: 1052 - protein, 15 aa, Artificial Sequence
  Made in a lab
EPKSCDKTCCCPPCP

SEQ ID NO: 1053 - protein, 15 aa, Artificial Sequence
  Made in a lab
EPKSCPPPPPCPPCP

SEQ ID NO: 1054 - protein, 15 aa, Artificial Sequence
  Made in a lab
PPPPCDKTHTCPPCP

SEQ ID NO: 1055 - protein, 15 aa, Artificial Sequence
  Made in a lab
EPKSCDKTHTCWWCP

SEQ ID NO: 1056 - protein, 15 aa, Artificial Sequence
  Made in a lab
EPKSCDWWHTCPPCP

SEQ ID NO: 1057 - protein, 15 aa, Artificial Sequence
  Made in a lab
EPKCSDKTHTCPPCP

SEQ ID NO: 1058 - protein, 15 aa, Artificial Sequence
  Made in a lab
EPKSDCKTHTCPPCP

SEQ ID NO: 1059 - protein, 15 aa, Artificial Sequence
  Made in a lab
EPKSDCWWHTCPPCP

SEQ ID NO: 1060 - protein, 15 aa, Artificial Sequence
  Made in a lab
EPKSCDFFHTCPPCP

SEQ ID NO: 1061 - protein, 15 aa, Artificial Sequence
  Made in a lab
EPKSCDWWWTCPPCP

SEQ ID NO: 1062 - protein, 15 aa, Artificial Sequence
  Made in a lab
EPKSCWWTHTCPPCP

SEQ ID NO: 1063 - protein, 15 aa, Artificial Sequence
  Made in a lab
EPWWCDKTHTCPPCP

SEQ ID NO: 1064 - protein, 16 aa, Artificial Sequence
  Made in a lab
SQPEIVPISCPPCPNS

SEQ ID NO: 1065 - protein, 17 aa, Artificial Sequence
  Made in a lab
TGEPKSSDKTHTCPPCP

SEQ ID NO: 1066 - protein, 17 aa, Artificial Sequence
  Made in a lab
EPKSTDKTHTCPPCPNS

SEQ ID NO: 1067 - protein, 17 aa, Artificial Sequence
  Made in a lab
EPKSTDKTHTSPPSPNS

SEQ ID NO: 1068 - protein, 17 aa, Artificial Sequence
  Made in a lab
EPKSTDKTHTCPPCPNS

SEQ ID NO: 1069 - protein, 17 aa, Artificial Sequence
  Made in a lab
EPKSSDKTHTCPPCPNS

SEQ ID NO: 1070 - protein, 18 aa, Artificial Sequence
  Made in a lab
EPKSCDKTHTCPGGGPCP

SEQ ID NO: 1071 - protein, 18 aa, Artificial Sequence
  Made in a lab
EPKSCDGGGKTHTCPPCP

SEQ ID NO: 1072 - protein, 18 aa, Artificial Sequence
  Made in a lab
EPKSCDPPPKTHTCPPCP

SEQ ID NO: 1073 - protein, 18 aa, Artificial Sequence
  Made in a lab
EPKSCDKTHTCPPPPPCP

SEQ ID NO: 1074 - protein, 18 aa, Artificial Sequence
  Made in a lab
GGGGSGGGGSGGGGSGNS

SEQ ID NO: 1075 - protein, 19 aa, Artificial Sequence
  Made in a lab
NYGGGGSGGGGSGGGGSNS

SEQ ID NO: 1076 - protein, 19 aa, Artificial Sequence
  Made in a lab
LSVKADFLTPSISPPCPNS

SEQ ID NO: 1077 - protein, 19 aa, Artificial Sequence
  Made in a lab
SVLANFSQPEISCPPCPNS

SEQ ID NO: 1078 - protein, 20 aa, Artificial Sequence
  Made in a lab
GQRHNNSSLNTRTQKARHSP

SEQ ID NO: 1079 - protein, 20 aa, Artificial Sequence
  Made in a lab
LSVLANFSQPEISCPPCPNS

SEQ ID NO: 1080 - protein, 20 aa, Artificial Sequence
  Made in a lab
LKIQERVSKPKISCPPCPNS

SEQ ID NO: 1081 - protein, 20 aa, Artificial Sequence
  Made in a lab
REQLAEVTLSLKACPPCPNS

SEQ ID NO: 1082 - protein, 20 aa, Artificial Sequence
  Made in a lab
RIHQMNSELSVLACPPCPNS

SEQ ID NO: 1083 - protein, 20 aa, Artificial Sequence
  Made in a lab
RIHLNVSERPFPPCPPCPNS

SEQ ID NO: 1084 - protein, 20 aa, Artificial Sequence
  Made in a lab
NSLFNQEVQIPLTESYCPNS

SEQ ID NO: 1085 - protein, 20 aa, Artificial Sequence
  Made in a lab
EEEEDEEDEEDEEEEEDGNS

SEQ ID NO: 1086 - protein, 21 aa, Artificial Sequence
  Made in a lab
LDVSERPFPPHIQSCPPCPNS

SEQ ID NO: 1087 - protein, 21 aa, Artificial Sequence
  Made in a lab
DTKGKNVLEKIFDSCPPCPNS

SEQ ID NO: 1088 - protein, 21 aa, Artificial Sequence
  Made in a lab
LPPETQESQEVTLSCPPCPNS

SEQ ID NO: 1089 - protein, 21 aa, Artificial Sequence
  Made in a lab
EPAFTPGPNIELQKDSDCPNS

SEQ ID NO: 1090 - protein, 21 aa, Artificial Sequence
  Made in a lab
QRHNNSSLNTRTQKARHCPNS

SEQ ID NO: 1091 - protein, 21 aa, Artificial Sequence
  Made in a lab
QRHNNSSLNTRTQKARHSPNS

SEQ ID NO: 1092 - protein, 36 aa, Artificial Sequence; identical to SEQ ID NO: 603
  Made in a lab

SEQ ID NO: 1093 - protein, 122 aa, Artificial Sequence; identical to SEQ ID NO: 604
  Made in a lab

SEQ ID NO: 1094 - protein, 13 aa, Artificial Sequence
  Made in a lab
GGGASCPPCAGNS

SEQ ID NO: 1095 - protein, 39 aa, Artificial Sequence
  Made in a lab
NNYGGGGSGGGGSGGGGSGNYGGGGSGGGGSGGGGSGNS

SEQ ID NO: 1096 - protein, 20 aa, Artificial Sequence
  Made in a lab
NYGGGGSGGGGSGGGGSGNS

SEQ ID NO: 1097 - protein, 7 aa, Artificial Sequence
  Made in a lab
SPPSPNS

SEQ ID NO: 1098 - protein, 17 aa, Artificial Sequence
  Made in a lab
EPTSTDKTHTSPPSPNS

SEQ ID NO: 1099 - protein, 17 aa, Artificial Sequence
  Made in a lab
EPTSTDKTHTCPPCPNS

SEQ ID NO: 1100 - protein, 19 aa, Artificial Sequence
  Made in a lab
LSVKADFLTPSISPPCPNS

SEQ ID NO: 1101 - protein, 12 aa, Artificial Sequence
  Made in a lab
GGGASCPPCANS

SEQ ID NO: 1102 - protein, 26 aa, Artificial Sequence
  Made in a lab
SGGGGSGGGGSGGGGSGGGGSGGGGS

SEQ ID NO: 1103 - protein, 16 aa, Artificial Sequence
  Made in a lab
GGGGSGGGGSGGGGAS

SEQ ID NO: 1104 - protein, 16 aa, Artificial Sequence
  Made in a lab
SEPKSSDKTHTCPPCP

SEQ ID NO: 1105 - protein, 18 aa, Artificial Sequence
  Made in a lab
GDQEPKSSDKTHTSPPCP

SEQ ID NO: 1106 - protein, 21 aa, Artificial Sequence
  Made in a lab
QRHNNSSLNTGTQMAGHSPNS

(19) United States
(12) Patent Application Publication
(10) Pub. No.: US 2013/0052195 A1
(43) Pub. Date: Feb. 28, 2013

(54) Compositions Comprising TNF-alpha and IL-6 Antagonists and Methods of Use Thereof

(71) Applicants: Lynda E. Misher, Seattle, WA (US); Alan Keith Lofquist, Kirkland, WA (US)

(72) Inventors: Lynda E. Misher, Seattle, WA (US); Alan Keith Lofquist, Kirkland, WA (US)

(73) Assignee: Emergent Product Development Seattle,LLC, Seattle, WA (US)

(21) Appl. No.: 13/518,184

(22) PCT Filed: Dec. 23, 2010
(86) PCT No.: PCT/US10/62081

§ 371 Date: Oct. 31, 2012

Related U.S. Application Data
(60) Provisional application No. 61/289,929, filed on Dec. 23, 2009.

(51) Int. Cl. A61K 39/395; A61P 1/00; A61P 19/04; A61P 17/06; A61P 37/00; A61P 29/00

(52) U.S. Cl. 424/135.1; 424/158.1

(57) ABSTRACT

The present disclosure relates to compositions and methods for treating autoimmune diseases including rheumatoid arthritis. In particular, the present disclosure relates to compositions comprising IL-6 antagonists (e.g., anti-IL6 or anti-IL6R or anti-hyperIL6) and TNF-α antagonists (e.g., (anti-TNF or etanercept) and methods of using same in the treatment of rheumatoid arthritis.

### CROSS-REFERENCE TO RELATED APPLICATION

This application claims the benefit under 35 U.S.C. §119(e) of U.S. Provisional Patent Application No. 61/289,929, filed Dec. 23, 2009, which provisional application is incorporated herein by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 910180_{—}423PC_SEQUENCE_LISTING.txt. The text file is 460 KB, was created on Dec. 23, 2010, and is being submitted electronically via EFS-Web, concurrent with the filing of the specification.

### TECHNICAL FIELD

The present disclosure relates generally to compositions and methods for treating autoimmune diseases including rheumatoid arthritis, systemic lupus erythematosus (SLE), psoriasis and inflammatory bowel disease (IBD). In particular, the present disclosure relates to compositions comprising IL-6 antagonists (e.g., anti-IL6 or anti-IL6R or anti-hyperlL6) and TNF-α antagonists (e.g., (anti-TNF or etanercept) and methods of using same in the treatment of rheumatoid arthritis.

### BACKGROUND OF THE DISCLOSURE

Interleukin 6 (“IL6”) is a pleiotropic cytokine that regulates host immune responses, inflammation, hematopoiesis, and oncogenesis. IL6 biology is mediated by a multicomponent molecular system with two distinct modes of signaling operative on overlapping but non-identical cell populations. These are referred to as cis-signaling (also known as “classical” signaling) and trans-signaling.

In cis-signaling, IL6 binds to cell surface IL6 receptor, the ligand binding part of IL6R that is referred to as IL6Rα or CD126 (previously called gp80). The cell-bound IL6|IL6Rα complex in turn binds to non-ligand binding but signal transducing membrane protein gp130 (also known as IL6ST, IL6Rβ, or CD130), which induces gp130 dimerization and initiation of signaling. Thus, cis-signaling is restricted to the subset of cell types that express cell-surface IL6Rα, which is generally limited to, for example, mitogen-activated B cells, T cell subsets, peripheral monocytes, and certain tumors. The resultant ternary complex on the cell surface assembles into a very stable hexamer with a 2:2:2 ratio of IL6:IL6Rα:gp130 (Boulanger et al. (2003) Science 300:2101).

In trans-signaling, soluble IL6Rα (“sIL6Rα”) complexes with IL6 and the resulting circulating sIL6xR complex can bind to and activate any gp130-expressing cell (but not cells also expressing IL6Rα, Taga et al. (1989) Cell 58:573). Many, perhaps all or nearly all, cells in the human body express gp130. Because gp130 is ubiquitous, trans-signaling can affect many cell types and thereby sometimes cause disease.

The membrane protein gp130 also exists in soluble form (“sgp130”), which can bind sIL6xR complex in circulation. But, the sIL6xR complex binds equally well to membrane and soluble gp130 (see Jones et al., (2005) J. Interferon Cytokine Res. 25:241). Therefore, a molar excess of sgp130 can inhibit trans-signaling (by reducing the amount of available sIL6xR complex in circulation), which will not significantly affecting cis-signaling because the affinity of sgp130 is orders of magnitude less, as compared to cell surface gp130, for cell-bound IL6|IL6Rα complex (see, e.g., Jostock et al. (2001) Eur. J. Biochem. 268:160). Thus, it has been suggested that spg130 may be useful in inhibiting IL6 activity (see, e.g., Jostock et al. (2001) Eur. J. Biochem. 268:160). But, in addition to IL6, gp130 is a common signal-transducing protein for a family of gp130 cytokines. These include leukemia inhibitory factor (LIF), ciliary neurotrophic factor (CNTF), neuropoietin (NP), cardiotropin like cytokine (CLC), oncostatin M (OSM), IL-27, IL-31 and cardiotrophin-1 (CT-1). Hence, although sgp130 can inhibit trans-signaling, administering such a compound to patients may have some unintended adverse effects.

Increased production of IL6 has been implicated in various disease processes, including Alzheimer's disease, autoimmunity (e.g., rheumatoid arthritis, SLE), inflammation, myocardial infarction, Paget's disease, osteoporosis, solid tumors (e.g., colon cancer, RCC prostatic and bladder cancers), certain neurological cancers, B-cell malignancies, such as Castleman's disease, some lymphoma subtypes, CLL, and, in particular, multiple myeloma. In some instances, IL-6 is implicated in proliferation pathways because it acts with other factors, such as heparin-binding epithelial growth factor and hepatocyte growth factor.

Several IL6 and IL6Rα antibody antagonists are known. For example, for IL6, Way et al. (US Patent Application Publication No. 2007/0178098) disclose antibodies against IL6 to sterically block IL6 or sIL6xR complex from binding to gp130 (see also U.S. Pat. No. 7,291,721). For example, for IL6Rα, Kishimoto (U.S. Pat. No. 5,670,373) discloses antibodies against IL6Rα that inhibit IL6 activity.

Tumor Necrosis Factor Receptor (TNFR), also known as CD120 or cachectin, is a member of the tumor necrosis factor receptor superfamily and is the receptor for Tumor Necrosis Factor-α (TNF-α). There are two variants of this cytokine receptor, TNFR1 and TNFR2 (CD120a and CD120b receptors, respectively). TNFR1 has a molecular weight of about 55 KD and is therefore sometimes referred to as p55. TNFR2 has a molecular weight of about 75 KD and is therefore sometimes referred to as p75.

A majority of cell types and tissues appear to express both TNF receptors. Both exist on the cell surface as well as in soluble forms and both are active in signal transduction, although they can mediate distinct cellular responses. TNFR1 appears to be responsible for signaling most TNF responses. Among other activities, TNFR2 stimulates thymocyte proliferation, activates NF-κβ, and is an accessory to TNFR1 in the signaling of responses primarily mediated by TNFR1, such as cytotoxicity.

TNF antagonists, such as anti-TNF antibodies, can positively affect various inflammatory conditions. For example, infliximab is indicated in the United States for the treatment of rheumatoid arthritis, Crohn's disease, ankylosing spondylitis, psoriatic arthritis, plaque psoriasis, and ulcerative colitis. According to REMICADE® (infliximab) prescribing information, biological activities attributed to TNF include induction of pro-inflammatory cytokines such as interleukins (IL) 1 and 6, enhancement of leukocyte migration by increasing endothelial layer permeability and expression of adhesion molecules by endothelial cells and leukocytes, activation of neutrophil and eosinophil functional activity, induction of acute phase reactants and other liver proteins, as well as tissue degrading enzymes produced by synoviocytes and/or chondrocytes. Recently, perispinal delivery of the TNF-α inhibitor etanercept has been shown to reduce symptoms in patients with Alzheimer's disease (Tobinick and Gross (2008) BMC Neurol. 8:27-36; Griffin (2008) J. Neuroinflammation, 5:3-6).

One aspect of the disclosure provides a method for treating rheumatoid arthritis, SLE, psoriasis, or inflammatory bowel disease (including Crohn's Disease and ulcerative colitis), comprising administering to a subject in need thereof a therapeutically effective amount of a combination of an IL6 antagonist and a TNF-α antagonist, which can be administered concurrently or sequentially or a combination thereof. In certain embodiments, the combination comprises an isolated IL6 antagonist comprising a binding domain specific for an IL6/IL6R (IL6xR) complex, wherein the binding domain specific for an IL6/IL6R (IL6xR) complex comprises an amino acid sequence that is at least 80% identical to one or more light chain variable regions as listed in SEQ ID NOs:373-434 and 799-804 and an amino acid sequence that is at least 80% identical to one or more of the heavy chain variable regions as listed in SEQ ID

NOs:435-496 and 805-810, and an isolated TNF-α antagonist comprising a binding domain specific for TNF-α, wherein the binding domain specific for TNF-α comprises amino acids 23-257 of SEQ ID NO:846 or amino acids 31-211 of SEQ ID NO:847. In one embodiment, the isolated 1L6 antagonist and the isolated TNF-α antagonist are both an antibody, or an antigen binding domain thereof, a Fab, or a scFv. In another embodiment, the isolated IL6 antagonist is an antibody and the isolated TNF-α antagonist is a small modular immunopharmaceutical (SMIP™). In a further embodiment, the isolated IL6 antagonist is a SMIP™ protein and the isolated TNF-α antagonist is an antibody. In yet another embodiment, the isolated isolated IL6 antagonist and the isolated TNF-α antagonist are both SMIP™ proteins. In a further embodiment, the binding domain of the isolated IL6 antagonist and the isolated TNF-α antagonist are an antibody or antigen binding domain thereof, a Fab, or a scFv. In this regard, the binding domain of the isolated IL6 antagonist may comprise a light chain variable region containing CDR1, CDR2, and CDR3 sequences that are each at least 80% identical to at least one light chain variable region CDR1, CDR2, and CDR3 as set forth in SEQ ID NOS:373-434 and 799-804, respectively. In one particular embodiment, the binding domain of the isolated IL6 antagonist comprises a heavy chain variable region containing CDR1, CDR2, and CDR3 sequences that are each at least 80% identical to at least one heavy chain variable region CDR1, CDR2, and CDR3 as set forth in SEQ ID NOS:435-496 and 805-810, respectively. In another embodiment, the binding domain of the isolated IL6 antagonist comprises a light chain variable region containing CDR1, CDR2, and CDR3 sequences that are each at least 80% identical to at least one light chain variable region CDR1, CDR2, and CDR3 as set forth in SEQ ID NOS:373-434 and 799-804, respectively, and comprises a heavy chain variable region containing CDR1, CDR2, and CDR3 sequences that are each at least 80% identical to at least one heavy chain variable region CDR1, CDR2, and CDR3 as set forth in SEQ ID NOS:435-496 and 805-810, respectively. In a further embodiment, the IL6xR complex has an amino acid sequence as set forth in SEQ ID NO:606. In certain embodiments, this disclosure provides a method for treating rheumatoid arthritis, SLE, psoriasis, or inflammatory bowel disease (including Crohn's Disease and ulcerative colitis), comprising administering to a subject in need thereof a therapeutically effective amount of a composition comprising an IL6 antagonist and a TNF-α antagonist.

In further embodiments of this disclosure, the binding domains of the isolated IL6 antagonist and the isolated TNF-α antagonist are fused to (a) an immunoglobulin Fc domain or one or more CH domains of an immunoglobulin Fc domain, or (b) a serum protein binding protein. In this regard, the one or more CH domains of an immunoglobulin Fc domain may comprise a CH2 constant region and CH3 constant region, preferably IgG1 CH2 and CH3 domains. In one particular embodiment, I) the isolated IL6 antagonist comprises, from amino-terminus to carboxy-terminus, (a) the binding domain specific for IL6xR fused to a linker, (b) the linker fused to an immunoglobulin heavy chain CH2 constant region polypeptide, and (c) the CH2 constant region polypeptide fused to an immunoglobulin heavy chain CH3 constant region polypeptide; and II) the isolated TNF-α antagonist comprises, from amino-terminus to carboxy-terminus, (a) the binding domain specific for TNF-α fused to a linker, (b) the linker fused to an immunoglobulin heavy chain CH2 constant region polypeptide, and (c) the CH2 constant region polypeptide fused to an immunoglobulin heavy chain CH3 constant region polypeptide. In another embodiment, I) the isolated IL6 antagonist comprises, from carboxy-terminus to amino-terminus, (a) the polypeptide binding domain specific for IL6xR fused to a first linker, (b) the first linker fused to an immunoglobulin heavy chain CH3 constant region polypeptide, (c) the CH3 constant region polypeptide fused to an immunoglobulin heavy chain CH2 constant region polypeptide, and (d) the CH2 constant region polypeptide fused to a second linker; and II) the isolated TNF-α antagonist comprises, from carboxy-terminus to amino-terminus, (a) the binding domain specific for TNF-α fused to a first linker, (b) the first linker fused to an immunoglobulin heavy chain CH3 constant region polypeptide, (c) the CH3 constant region polypeptide fused to an immunoglobulin heavy chain CH2 constant region polypeptide, and (d) the CH2 constant region polypeptide fused to a second linker. In further embodiments, the linker is an immunoglobulin hinge region polypeptide. In one particular embodiment, the linker is selected from the group consisting of SEQ ID NO:497-604, 823-828, 830-845, and 851-1106.

### BRIEF DESCRIPTION OF THE DRAWINGS

FIG. 1: Zeu080a+anti-mIL6 Ab—Mediated Blockade of m TNF-α-Induced Biomarker Response. Serum concentration of mouse serum amyloid A (SAA) was measured in mice in each group as set forth in Table 1 at 2 hours (Panel A) and 24 hours (Panel B) post TNF-α injection.

FIG. 2: Zeu080a+anti-mIL6 Ab—Mediated Blockade of mTNF-α-Induced Biomarker Response. Mouse serum concentration of TNF-α was measured at the 2-hour time point post TNF-α injection.

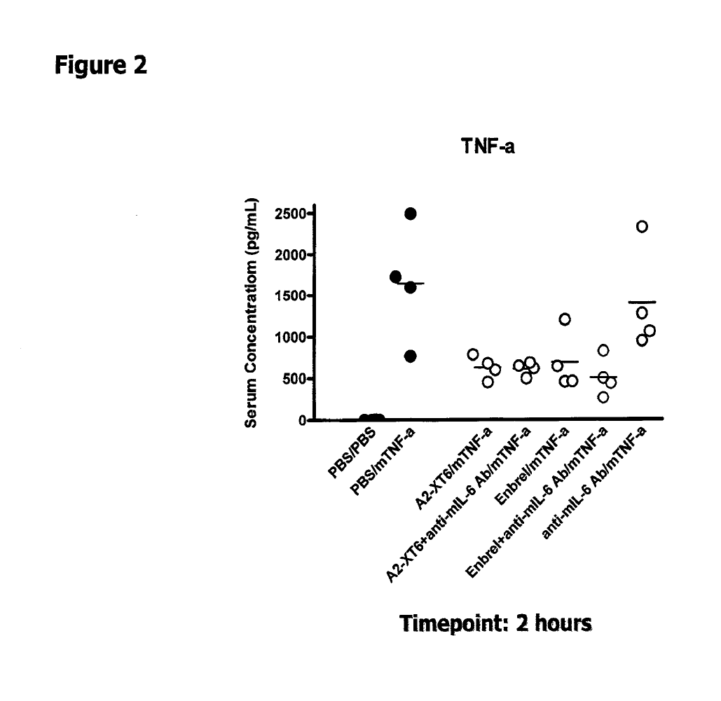

FIG. 3: In vivo treatment with ENBREL® fusion protein (etanercept) in combination with anti-mIL6 antibody reduces severity of arthritis in a mouse model. The figure shows a graph of mean arthritis scores over time following various treatments. Note that curves for individual groups end when the first mouse was euthanized due to severe arthritis. Maximum score is 16.

FIG. 4: In vivo treatment with ENBREL® in combination with anti-mIL6 antibody reduces weight loss in a mouse model of arthritis. The figure shows a graph of mean body weights over time through day 24 following various treatments. Primed mice were inoculated with 50 μg G6PI and 25 μg Mtb.

FIG. 5: In vivo treatment with ENBREL® in combination with anti-mIL6 antibody reduces weight loss in a mouse model of arthritis. The figure shows a graph of mean body weights over time through day 6 (expanded from FIG. 4) following various treatments. Primed mice were inoculated with 50 μg G6PI and 25 μg Mtb.

### DETAILED DESCRIPTION

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents defines a term that contradicts the term's definition in the application, the definition that appears in this application controls.

The present disclosure relates generally to compositions comprising a combination of a TNF-α antagonist and an IL6 antagonist. In this regard, the present disclosure provides that administering a combination of the two antagonists is much more effective (synergistic) for reducing arthritis in a mouse model than administering either antagonist alone. Accordingly, the compositions of the present disclosure may be used in a variety of therapeutic settings for the treatment of SLE, psoriasis, or inflammatory bowel disease (including Crohn's Disease and ulcerative colitis), which are autoimmune diseases associated with TNF-α, and IL6 activity.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, “about” or “consisting essentially of mean ±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include” and “comprise” are used synonymously. In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present disclosure.

A “binding domain” or “binding region” according to the present disclosure may be, for example, any protein, polypeptide, oligopeptide, or peptide that possesses the ability to specifically recognize and bind to a biological molecule (e.g., TNF-α or IL6) or complex of more than one of the same or different molecule or assembly or aggregate, whether stable or transient (e.g., IL6/IL6R complex). Such biological molecules include proteins, polypeptides, oligopeptides, peptides, amino acids, or derivatives thereof, lipids, fatty acids, or derivatives thereof; carbohydrates, saccharides, or derivatives thereof; nucleotides, nucleosides, peptide nucleic acids, nucleic acid molecules, or derivatives thereof; glycoproteins, glycopeptides, glycolipids, lipoproteins, proteolipids, or derivatives thereof; other biological molecules that may be present in, for example, a biological sample; or any combination thereof. A binding region includes any naturally occurring, synthetic, semi-synthetic, or recombinantly produced binding partner for a biological molecule or other target of interest. A variety of assays are known for identifying binding domains of the present disclosure that specifically bind with a particular target, including Western blot, ELISA, or BIACORE® surface plasmon resonance analysis.

Binding domains and fusion proteins thereof of this disclosure are capable of binding to a desired degree, including “specifically or selectively binding” a target while not significantly binding other components present in a test sample, if they bind a target molecule with an affinity or Kₐ (i.e., an equilibrium association constant of a particular binding interaction with units of 1/M) of, for example, greater than or equal to about 10⁵ M⁻¹, 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding domains refers to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater. Alternatively, affinity may be defined as an equilibrium dissociation constant (K_{d}) of a particular binding interaction with units of M (e.g., 10⁻⁵ M to 10⁻¹³ M). Affinities of binding domain polypeptides and fusion proteins according to the present disclosure can be readily determined using conventional techniques (see, e.g., Scatchard et al. (1949) Ann. N.Y. Acad. Sci. 51:660; U.S. Pat. Nos. 5,283,173; 5,468,614; BIACORE® analysis; or the equivalent).

Binding domains of this disclosure can be generated as described herein or by a variety of methods known in the art (see, e.g., U.S. Pat. Nos. 6,291,161; 6,291,158). Sources include antibody gene sequences from various species (which can be formatted as antibodies, sFvs, scFvs or Fabs, such as in a phage library), including human, camelid (from camels, dromedaries, or llamas; Hamers-Casterman et al. (1993) Nature, 363:446 and Nguyen et al. (1998) J. Mol. Biol., 275:413), shark (Roux et al. (1998) Proc. Nat'l. Acad. Sci. (USA) 95:11804), fish (Nguyen et al. (2002) Immunogenetics, 54:39), rodent, avian, ovine, sequences that encode random peptide libraries or sequences that encode an engineered diversity of amino acids in loop regions of alternative non-antibody scaffolds, such as fibrinogen domains (see, e.g., Weisel et al. (1985) Science 230:1388), Kunitz domains (see, e.g., U.S. Pat. No. 6,423,498), lipocalin domains (see, e.g., WO 2006/095164), V-like domains (see, e.g., US Patent Application Publication No. 2007/0065431), C-type lectin domains (Zelensky and Gready (2005) FEBS J. 272:6179), mAb² or FCAB™ (see, e.g., PCT Patent Application Publication Nos. WO 2007/098934; WO 2006/072620), or the like. Additionally, traditional strategies for hybridoma development using a synthetic single chain IL6/IL6R complex, such as a human IL6/IL6R complex or Hyper-IL6 (IL6 joined by a peptide linker IL6R), as an immunogen in convenient systems (e.g., mice, HUMAB MOUSE®, TC MOUSE™, KM-MOUSE®, llamas, chicken, rats, hamsters, rabbits, etc.) can be used to develop binding domains of this disclosure.

Terms understood by those in the art as referring to antibody technology are each given the meaning acquired in the art, unless expressly defined herein. For example, the terms “V_{L}” and “V_{H}” refer to the variable binding region derived from an antibody light and heavy chain, respectively. The variable binding regions are made up of discrete, well-defined sub-regions known as “complementarity determining regions” (CDRs) and “framework regions” (FRs). The terms “C_{L}” and “C_{H}” refer to an “immunoglobulin constant region,” i.e., a constant region derived from an antibody light or heavy chain, respectively, with the latter region understood to be further divisible into C_{H1}, C_{H2}, C_{H3} and C_{H4} constant region domains, depending on the antibody isotype (IgA, IgD, IgE, IgG, IgM) from which the region was derived. A portion of the constant region domains makes up the Fc region (the “fragment crystallizable” region), which contains domains responsible for the effector functions of an immunoglobulin, such as ADCC (antibody-dependent cell-mediated cytotoxicity), ADCP (antibody-dependent cell-mediated phagocytosis), CDC (complement-dependent cytotoxicity) and complement fixation, binding to Fc receptors, greater half-life in vivo relative to a polypeptide lacking an Fc region, protein A binding, and perhaps even placental transfer (see Capon et al. (1989) Nature, 337:525). Further, a polypeptide containing an Fc region allows for dimerization or multimerization of the polypeptide. A “hinge region,” also referred to herein as a “linker,” is an amino acid sequence interposed between and connecting the variable binding and constant regions of a single chain of an antibody, which is known in the art as providing flexibility in the form of a hinge to antibodies or antibody-like molecules.

The domain structure of immunoglobulins is amenable to engineering, in that the antigen binding domains and the domains conferring effector functions may be exchanged between immunoglobulin classes and subclasses. Immunoglobulin structure and function are reviewed, for example, in Harlow et al., Eds., Antibodies: A Laboratory Manual, Chapter 14 (Cold Spring Harbor Laboratory, Cold Spring Harbor, 1988). An extensive introduction as well as detailed information about all aspects of recombinant antibody technology can be found in the textbook Recombinant Antibodies (John Wiley & Sons, NY, 1999). A comprehensive collection of detailed antibody engineering lab Protocols can be found in R. Kontermann and S. Dübel, Eds., The Antibody Engineering Lab Manual (Springer Verlag, Heidelberg/New York, 2000).

“Derivative” as used herein refers to a chemically or biologically modified version of an antagonist that is structurally similar to the parent antagonist and (actually or theoretically) derivable from that parent antagonist.

Generally, a “derivative” differs from an “analogue” in that a parent antagonist may be the starting material to generate a “derivative,” whereas the parent antagonist may not necessarily be used as the starting material to generate an “analogue.” An analogue may have different chemical, biological or physical properties of the parent antagonist. For example, a derivative may be more hydrophilic or it may have altered reactivity (e.g., a CDR having an amino acid change that alters its affinity for a target) as compared to the parent antagonist.

The term “biological sample” includes a blood sample, biopsy specimen, tissue explant, organ culture, biological fluid or any other tissue or cell or other preparation from a subject or a biological source. A subject or biological source may, for example, be a human or non-human animal, a primary cell culture or culture adapted cell line including genetically engineered cell lines that may contain chromosomally integrated or episomal recombinant nucleic acid sequences, somatic cell hybrid cell lines, immortalized or immortalizable cell lines, differentiated or differentiatable cell lines, transformed cell lines, or the like. In further embodiments of this disclosure, a subject or biological source may be suspected of having or being at risk for having a disease, disorder or condition, including a malignant disease, disorder or condition or a B cell disorder. In certain embodiments, a subject or biological source may be a hyperproliferative, inflammatory, or autoimmune disease, and in certain other embodiments of this disclosure the subject or biological source may be known to be free of a risk or presence of such disease, disorder, or condition.

In certain embodiments, the present disclosure makes possible the depletion or modulation of cells associated with aberrant TNF-α and/or IL6 activity by providing antagonist proteins that bind to TNF-α and antagonists that bind to IL6, IL6R, an IL6/IL6R complex, or any combination thereof.

The complex of IL6 with membrane or soluble 1L6 receptor (IL6Rα) is referred to herein as IL6xR when referring to IL6 with either membrane IL6Rα or soluble IL6Rα (sIL6Rα), and as sIL6xR when referring only to the complex of IL6 with sIL6Rα. In some embodiments, antagonist proteins containing a binding domain specific for IL6xR have one or more of the following properties: (1) have greater or equal affinity for an IL6xR complex than for IL6 alone or IL6Rα alone, or have greater affinity for IL6R═ alone or an IL6xR complex than for IL6 alone; (2) compete with membrane gp130 for binding with a sIL6xR complex or enhance soluble gp130 binding with a sIL6xR complex; (3) preferentially inhibit IL6 trans-signaling over IL6 cis-signaling; and (4) do not inhibit signaling of gp130 family cytokines other than IL6.

As used herein, a “hinge region” or a “hinge” refers to (a) an immunoglobulin hinge region (made up of, for example, upper and core regions) or a functional variant thereof, including wild type and altered immunoglobulin hinges, (b) a lectin interdomain region or a functional variant thereof, (c) a cluster of differentiation (CD) molecule stalk region or a functional variant thereof, or (d) a portion of a cell surface receptor (interdomain region) that connects immunoglobulin V-like or immunoglobulin C-like domains. “Junction amino acids” or “junction amino acid residues” refer to one or more (e.g., 2, 3, 4, 5, 6, 7, 8, 9, or 10, or more) amino acid residues between two adjacent regions or domains of a single chain polypeptide, such as between a hinge and an adjacent Fc region portion or between a hinge and an adjacent binding domain or between a peptide linker that links two immunoglobulin variable domains and an adjacent immunoglobulin variable domain. Junction amino acids may result from the construct design of a single chain polypeptide (e.g., amino acid residues resulting from the use of a restriction enzyme site during the construction of a nucleic acid molecule encoding a single chain polypeptide).

“Sequence identity,” as used herein, refers to the percentage of amino acid residues in one sequence that are identical with the amino acid residues in another reference polypeptide sequence after aligning the sequences and introducing gaps, if necessary, to achieve the maximum percent sequence identity, and not considering any conservative substitutions as part of the sequence identity. The percentage sequence identity values are generated by the NCBI BLAST2.0 software as defined by Altschul et al. (1997) “Gapped BLAST and PSI-BLAST: a new generation of protein database search programs,” Nucleic Acids Res. 25:3389-3402, with the parameters set to default values.

In certain embodiments, an altered immunoglobulin domain only contains conservative amino acid substitutions of a wild type immunoglobulin domain. In certain other embodiments, an altered immunoglobulin domain only contains non-conservative amino acid substitutions of a wild type immunoglobulin domain. In yet other embodiments, an altered immunoglobulin domain contains both conservative and non-conservative amino acid substitutions.

A “conservative substitution” is recognized in the art as a substitution of one amino acid for another amino acid that has similar properties. Exemplary conservative substitutions are well known in the art (see, e.g., WO 97/09433, page 10, published Mar. 13, 1997; Lehninger, Biochemistry, Second Edition; Worth Publishers, Inc. NY:N.Y. (1975), pp. 71-77; Lewin, Genes IV, Oxford University Press, NY and Cell Press, Cambridge, Mass. (1990), p. 8). In certain embodiments, a conservative substitution includes a leucine to serine substitution.

As used herein, unless otherwise provided, a position of an amino acid residue in a variable region of an immunoglobulin molecule is numbered according to the Kabat numbering convention (Kabat, Sequences of Proteins of Immunological Interest, 5^{th} ed. Bethesda, Md.: Public Health Service, National Institutes of Health (1991)), and a position of an amino acid residue in a constant region of an immunoglobulin molecule is numbered according to EU nomenclature (Ward et al., 1995 Therap. Immunol. 2:77-94).

“Treatment,” “treating” or “ameliorating” refers to either a therapeutic treatment or prophylactic/preventative treatment. A treatment is therapeutic if at least one symptom of disease in an individual receiving treatment improves or a treatment may delay worsening of a progressive disease in an individual, or prevent onset of additional associated diseases.

A “therapeutically effective amount (or dose)” or “effective amount (or dose)” of a specific binding molecule or compound refers to that amount of the compound sufficient to result in amelioration of one or more symptoms of the disease being treated in a statistically significant manner. When referring to an individual active ingredient, administered alone, a therapeutically effective dose refers to that ingredient alone. When referring to a combination, a therapeutically effective dose refers to combined amounts of the active ingredients that result in the therapeutic effect, whether administered serially or simultaneously.

The term “pharmaceutically acceptable” refers to molecular entities and compositions that do not produce allergic or other serious adverse reactions when administered using routes well known in the art.

A “patient in need” refers to a patient at risk of, or suffering from, a disease, disorder or condition that is amenable to treatment or amelioration with a polypeptide heterodimer or a composition thereof provided herein.

### TNF-α Antagonists

TNFRs are type I transmembrane proteins having an extracellular domain that contains three well ordered cysteine rich domains (CRD1, CRD2, CRD3) characteristic of the TNFR superfamily, and a fourth less well conserved, membrane proximal CRD (Banner et al. (1993) Cell 73:431). A TNF-α antagonist of this disclosure inhibits the inflammatory or hyperproliferative activity of TNF-α. The antagonist domains may block TNFR multimerization or TNF-α binding, or the domains may bind to components of the receptor system and block activity either by preventing ligand activity or by preventing the assembly of the receptor complex. Various TNF-α antagonists are known in the art, including anti-TNF antibodies, such as infliximab and adalimumab, and soluble TNF receptor (sTNFR). Such antibody antagonists bind and inhibit TNF-α, but do not significantly inhibit TNF-β. Anti-TNF antibodies, including monoclonal antibodies, can be prepared using known techniques and are known in the art (see, e.g., U.S. Pat. No. 6,509,015). A TNF-α antagonist of this disclosure can also comprise one or more TNF-α binding domains present in a TNFR ectodomain.

TNF-α antagonists contemplated include a TNFR extracellular domain or sub-domain, one or more TNFR CRD domains (such as CRD2 and CRD3), or TNF-α-specific antibody-derived binding domains (analogous to the IL6 or IL6xR complex-specific antibody-derived binding domain described herein). In some embodiments, a TNF-α antagonist may be an extracellular domain (“ectodomain”) of a TNFR, such as an ectodomain of TNFR1 or TNFR2. As used herein, a TNFR ectodomain refers to a sTNFR, one or more CRDs, or any combination thereof of the TNFR domains. In certain embodiments, a TNF-α antagonist comprises an amino-terminal portion of TNFR2 (also known as p75, TNFRSF1B), such as the first 257 amino acids of TNFR2 as set forth in GenBank Accession No. NP_{—}001057.1 (SEQ ID NO:846). In other embodiments, a TNF-α antagonist comprises amino acids 23-257 of SEQ ID NO:846 (i.e., without the native leader sequence). In preferred embodiments, a TNF-α antagonist comprises a fragment of TNFR2 (e.g., an ectodomain), such amino acids 23-163 of SEQ ID NO:846 or amino acids 23-185 of SEQ ID NO:846 or amino acids 23-235 of SEQ ID NO:846. In other preferred embodiments, a TNF-α antagonist comprises a derivative of a TNFR2 fragment, such amino acids 23-163 of SEQ ID NO:846 with a deletion of amino acid glutamine at position 109 or amino acids 23-185 of SEQ ID NO:846 with a deletion of amino acid glutamine at position 109 and a deletion of amino acid proline at position 109 or amino acids 23-235 of SEQ ID NO:846 with a deletion of amino acid glutamine at position 109, a deletion of amino acid proline at position 109, and an substitution of amino acid aspartate at position 235 (to, for example, a threonine, alanine, serine, or glutamate). In further embodiments, a TNF-α antagonist comprises an amino-terminal portion of TNFR1 (also known as p55, TNFRSF1A), such as the first 211 amino acids of TNFR1 as set forth in GenBank Accession No. NP_{—}001056.1 (SEQ ID NO:847). In other embodiments, a TNF-α antagonist comprises amino acids 31-211 of SEQ ID NO:847 (i.e., without the native leader sequence).

Thus, a TNF-α antagonist can comprise an ectodomain of TNFRSF1A as set forth in SEQ ID NO:696 (with or without the native leader peptide sequence included in this sequence) or an ectodomain of TNFRSF1B as set forth in SEQ ID NO:695 (with or without the native leader peptide sequence included in this sequence).

In one aspect, a TNF-α antagonist or fusion protein thereof of this disclosure is specific for TNF-α wherein it has an affinity with a dissociation constant (K_{d}) of about 10⁻⁵ M to 10⁻¹³ M, or less. In certain embodiments, the TNF-α antagonist or fusion protein thereof binds TNF-α with an affinity that is less than about 300 pM. Another measure, the kinetic dissociation (k_{d}), also referred to herein as k_{OFF}, is a measure of the rate of complex dissociation and, thus, the ‘dwell time’ of the target molecule bound by a polypeptide binding domain of this disclosure. The k_{d} (k_{OFF}) has units of 1/sec. Exemplary TNF-α antagonists of this disclosure can have a k_{OFF} of about 10⁻⁴/sec (e.g., about a day) to about 10⁻⁸/sec or less. In certain embodiments, the k_{OFF} can range from about 10⁻¹/sec, about 10⁻²/sec, about 10⁻³/sec, about 10⁻⁴/sec, about 10⁻⁵/sec, about 10⁻⁶/sec, about 10⁻⁷/sec, about 10⁻⁸/sec, about 10⁻⁹/sec, about 10⁻¹⁰/sec, or less (see Graff et al. (2004) Protein Eng. Des. Sel. 17:293). In some embodiments, a TNF-α antagonist or fusion protein thereof of this disclosure will bind TNF-α with higher affinity and have a lower k_{OFF} rate as compared to the cognate TNFR binding to TNF-α. In further embodiments, a TNF-α antagonist or fusion protein thereof of this disclosure that blocks or alters TNF-α dimerization or other cell surface activity may have a more moderate affinity (i.e., a K_{d} of about 10⁻⁸ M to about 10⁻⁹ M) and a more moderate off rate (i.e., a k_{OFF} closer to about 10⁻⁴/sec) as compared to the affinity and dimerization rate of cognate TNFR.

Exemplary binding domains that function as TNF-α antagonists of this disclosure can be generated as described herein or by a variety of methods known in the art (see, e.g., U.S. Pat. Nos. 6,291,161, 6,291,158). Sources include antibody gene sequences from various species (which can be formatted as scFvs or Fabs, such as in a phage library), including human, camelid (from camels, dromedaries, or llamas; Hamers-Casterman et al. (1993) Nature, 363:446 and Nguyen et al. (1998) J. Mol. Biol., 275:413), shark (Roux et al. (1998) Proc. Nat'l. Acad. Sci. (USA) 95:11804), fish (Nguyen et al. (2002) Immunogenetics, 54:39), rodent, avian, ovine, sequences that encode random peptide libraries or sequences that encode an engineered diversity of amino acids in loop regions of alternative non-antibody scaffolds, such as fibrinogen domains (see, e.g., Weisel et al. (1985) Science 230:1388), Kunitz domains (see, e.g., U.S. Pat. No. 6,423,498), lipocalin domains (see, e.g., WO 2006/095164), V-like domains (see, e.g., US Patent Publication No. 2007/0065431), C-type lectin domains (Zelensky and Gready (2005) FEBS J. 272:6179), or the like. Additionally, traditional strategies for hybridoma development using a synthetic TNF-α or single chain TNFR ectodomain as an immunogen in convenient systems (e.g., mice, HUMAB MOUSE®, TC mouse™, KM-MOUSE®, llamas, chicken, rats, hamsters, rabbits, etc.) can be used to develop binding domains of this disclosure.

In an illustrative example, TNF-α antagonists of this disclosure specific for an TNF-α or single chain TNFR ectodomain can be identified using a Fab phage library of fragments (see, e.g., Hoet et al. (2005) Nature Biotechnol. 23:344) by screening for binding to a synthetic or recombinant TNF-α (using an amino acid sequence or fragment thereof as set forth in Gen Bank Accession No. NP_{—}000585.2) or single chain TNFR ectodomain. A TNF-α or a single chain TNFR ectodomain, as described herein or known in the art, can be used for such a screening. In certain embodiments, a TNF-α or single chain TNFR ectodomain used to generate a TNF-α antagonist can further comprise an intervening domain or a dimerization domain, as described herein, such as an immunoglobulin Fc domain or fragment thereof.

In some embodiments, TNF-α antagonist domains of this disclosure comprise V_{H} and V_{L} domains as described herein, such as those from infliximab or adalimumab. In certain embodiments, the V_{H} and V_{L} domains are rodent (e.g., mouse, rat), humanized, or human. In further embodiments, there are provided TNF-α antagonist domains of this disclosure that have a sequence that is at least 80%, at least 85%, at least 90%, at least 91%, at least 92%, at least 93%, at least 94%, at least 95%, at least 96%, at least 97%, at least 98%, at least 99%, at least 99.5%, or at least 100% identical to the amino acid sequence of one or more light chain variable regions (V_{L}) or to one or more heavy chain variable regions (V_{H}), or both, wherein each CDR has at most three amino acid substitutions (for instance, substitutions may be in the framework regions).

The terms “identical” or “percent identity,” in the context of two or more polypeptide or nucleic acid molecule sequences, means two or more sequences or subsequences that are the same or have a specified percentage of amino acid residues or nucleotides that are the same over a specified region (e.g., 60%, 65%, 70%, 75%, 80%, 85%, 90%, 91%, 92%, 93%, 94%, 95%, 96%, 97%, 98%, 99%, or 100% identity or 60%, 65%, 70%, 75%, 80%, 85%, 90%, 91%, 92%, 93%, 94%, 95%, 96%, 97%, 98%, 99%, or 100% identical), when compared and aligned for maximum correspondence over a comparison window, or designated region, as measured using methods known in the art, such as a sequence comparison algorithm, by manual alignment, or by visual inspection. For example, preferred algorithms suitable for determining percent sequence identity and sequence similarity are the BLAST and BLAST 2.0 algorithms, which are described in Altschul et al. (1977) Nucleic Acids Res. 25:3389 and Altschul et al. (1990) J. Mol. Biol. 215:403, respectively.

In any of these or other embodiments described herein, the V_{L} and V_{H} domains may be arranged in either orientation and may be separated by about a five to about a thirty amino acid linker as disclosed herein or any other amino acid sequence capable of providing a spacer function compatible with interaction of the two sub-binding domains. In certain embodiments, a linker joining the V_{H} and V_{L} domains comprises an amino acid sequence as set forth in SEQ ID NO:497-604, 823-828, 830-845, and 851-1106, such as Linker 46, (SEQ ID NO:542), Linker 47 (SEQ ID NO:543), or Linker 80 (SEQ ID NO:576). Multi-specific binding domains will have at least two specific sub-binding domains, by analogy to camelid antibody organization, or at least four specific sub-binding domains, by analogy to the more conventional mammalian antibody organization of paired V_{H} and V_{L} chains.

In further embodiments, TNF-α antagonist domains and fusion proteins may comprise a binding domain including one or more complementarity determining region (“CDR”), or multiple copies of one or more such CDRs, which have been obtained, derived, or designed from variable regions of an anti-TNF-α (such as infliximab or adalimumab) or anti-TNFR scFv or Fab fragment or from heavy or light chain variable regions thereof.

CDRs are defined in various ways in the art, including the Kabat, Chothia, AbM, and contact definitions. The Kabat definition is based on sequence variability and is the most commonly used definition to predict CDR regions (Johnson et al. (2000) Nucleic Acids Res. 28:214). The Chothia definition is based on the location of the structural loop regions (Chothia et al. (1986) J. Mol. Biol. 196:901; Chothia et al. (1989) Nature 342:877). The AbM definition, a compromise between the Kabat and Chothia definitions, is an integral suite of programs for antibody structure modeling produced by the Oxford Molecular Group (Martin et al. (1989) Proc. Nat'l. Acad. Sci. (USA) 86:9268; Rees et al., ABMTM, a computer program for modeling variable regions of antibodies, Oxford, UK; Oxford Molecular, Ltd.). An additional definition, known as the contact definition, has been introduced more recently (see MacCallum et al. (1996) J. Mol. Biol. 5:732), which is based on an analysis of available complex crystal structures.

By convention, the CDR domains in the heavy chain are referred to as H1, H2, and H3, which are numbered sequentially in order moving from the amino terminus to the carboxy terminus. The CDR-H1 is about ten to 12 residues in length and starts four residues after a Cys according to the Chothia and AbM definitions, or five residues later according to the Kabat definition. The H1 can be followed by a Trp, Trp-Val, Trp-Ile, or Trp-Ala. The length of H1 is approximately ten to 12 residues according to the AbM definition, while the Chothia definition excludes the last four residues. The CDR-H2 starts 15 residues after the end of H1 according to the Kabat and AbM definitions, which is generally preceded by sequence Leu-Glu-Trp-Ile-Gly (but a number of variations are known) and is generally followed by sequence Lys/Arg-Leu/Ile/Val/Phe/Thr/Ala-Thr/Ser/Ile/Ala. According to the Kabat definition, the length of H2 is about 16 to 19 residues, while the AbM definition predicts the length to be nine to 12 residues. The CDR-H3 usually starts 33 residues after the end of H2, is generally preceded by the amino acid sequence Cys-Ala-Arg and followed by the amino acid Gly, and has a length that ranges from three to about 25 residues.

By convention, the CDR regions in the light chain are referred to as L1, L2, and L3, which are numbered sequentially in order moving from the amino terminus to the carboxy terminus. The CDR-L1 generally starts at about residue 24 and generally follows a Cys. The residue after the CDR-L1 is always Trp, which begins one of the following sequences: Trp-Tyr-Gln, Trp-Leu-Gln, Trp-Phe-Gln, or Trp-Tyr-Leu. The length of CDR-L1 is approximately ten to 17 residues. The CDR-L2 starts about 16 residues after the end of L1 and will generally follow residues Ile-Tyr, Val-Tyr, Ile-Lys, or Ile-Phe. The CDR-L2 is about seven residues in length. The CDR-L3 usually starts 33 residues after the end of L2 and generally follows a Cys, which is generally followed by the sequence Phe-Gly-XXX-Gly and has a length of about seven to 11 residues.

Thus, a binding domain of this disclosure can comprise a single CDR from a variable region of an anti-TNF-α or anti-TNFR, or it can comprise multiple CDRs that can be the same or different. In certain embodiments, binding domains of this disclosure comprise V_{H} and V_{L} domains specific for a TNF-α or TNFR comprising framework regions and CDR1, CDR2 and CDR3 regions, wherein (a) the V_{H} domain comprises an amino acid sequence of a heavy chain CDR3; or (b) the V_{L} domain comprises an amino acid sequence of a light chain CDR3; or (c) the binding domain comprises a V_{H} amino acid sequence of (a) and a V_{L} amino acid sequence of (b); or the binding domain comprises a V_{H} amino acid sequence of (a) and a V_{L} amino acid sequence of (b) and wherein the V_{H} and V_{L} are found in the same reference sequence. In further embodiments, binding domains of this disclosure comprise V_{H} and V_{L} domains specific for an TNF-α or TNFR comprising framework regions and CDR1, CDR2 and CDR3 regions, wherein (a) the V_{H} domain comprises an amino acid sequence of a heavy chain CDR1, CDR2, and CDR3; or (b) the V_{L} domain comprises an amino acid sequence of a light chain CDR1, CDR2, and CDR3; or (c) the binding domain comprises a V_{H} amino acid sequence of (a) and a V_{L} amino acid sequence of (b); or the binding domain comprises a V_{H} amino acid sequence of (a) and a V_{L} amino acid sequence of (b), wherein the V_{H} and V_{L} amino acid sequences are from the same reference sequence.

In any of the embodiments described herein comprising specific CDRs, a binding domain can comprise (i) a V_{H} domain having an amino acid sequence that is at least 80%, 85%, 90%, 91%, 92%, 93%, 94%, 95%, 96%, 97%, 98%, or 99% identical to the amino acid sequence of a V_{H} domain, wherein each CDR has at most three amino acid changes (i.e., many of the changes will be in the framework regions); or (ii) a V_{L} domain having an amino acid sequence that is at least 80%, 85%, 90%, 91%, 92%, 93%, 94%, 95%, 96%, 97%, 98%, or 99% identical to the amino acid sequence of a V_{L} domain, wherein each CDR has at most three amino acid changes (i.e., many of the changes will be in the framework regions); or (iii) both a V_{H} domain of (i) and a V_{L} domain of (ii); or both a V_{H} domain of (i) and a V_{L} domain of (ii) wherein the V_{H} and V_{L} are from the same reference sequence.

A TNF-α binding domain antagonist of this disclosure may be an immunoglobulin-like domain such as an immunoglobulin scaffold. In certain embodiments, the TNF-α binding domain antagonist, such as an immunoglobulin-like domain, may be part of a fusion protein. Immunoglobulin scaffolds contemplated by this disclosure include, but are not limited to, a scFv, a domain antibody or a heavy chain-only antibody. In a scFv, this disclosure contemplates the heavy and light chain variable regions are joined by any linker peptide known in the art to be compatible with domain or region joinder in a binding molecule. Exemplary linkers are linkers based on the Gly₄Ser linker motif, such as (Gly₄Ser)ₙ, wherein n=1-6. If a binding domain of a fusion protein of this disclosure is based on a non-human immunoglobulin or includes non-human immunoglobulin CDRs, the binding domain may be “humanized” according to methods known in the art.

Alternatively, a TNF-α antagonist domain of fusion proteins of this disclosure may be a scaffold other than an immunoglobulin scaffold. Other scaffolds contemplated by this disclosure present the TNF-α-specific CDR(s) in a functional conformation. Other scaffolds contemplated include, but are not limited to, an A domain molecule, a fibronectin III domain, an anticalin, an ankyrin-repeat engineered binding molecule, an adnectin, a Kunitz domain or a protein AZ domain affibody.

In certain embodiments, a TNF-α or IL6 binding domain or protein of this disclosure is comprised in a SMIP™ or a reverse SMIP (PIMS) fusion protein. In this regard, the term SMIP refers to a highly modular compound class having enhanced drug properties over monoclonal and recombinant antibodies. SMIP proteins comprise a single polypeptide chain including a target-specific binding domain, based, for example, upon an antibody variable domain, in combination with a variable Fc region that permits the specific recruitment of a desired class of effector cells (such as, e.g., macrophages and natural killer (NK) cells) or recruitment of complement-mediated killing. Depending upon the choice of target and hinge regions, SMIP proteins can signal or block signaling via cell surface receptors. As used herein, engineered fusion proteins, termed “small modular immunopharmaceutical” or “SMIP products or proteins”, are as described in US Patent Publication Nos. 2003/133939, 2003/0118592, and 2005/0136049, and International Patent Publications WO02/056910, WO2005/037989, and WO2005/017148. In certain preferred embodiments, a SMIP protein comprises an scFv (linked VH and VL domains) from infliximab or adalimumab.

A PIMS molecule is a SMIP molecule in reverse orientation. In some embodiments, a TNF-α or IL6 binding domain may be comprised within a PIMS molecule such as those described in US Patent Publication No. 2009/0148447 and International Patent Publication W02009/023386.

### IL6 Antagonists

As noted above, in certain embodiments the present disclosure provides polypeptides containing a binding region or domain that is an IL6 antagonist (e.g., preferentially inhibits IL6 trans-signaling or inhibits both IL6 cis- and trans-signaling). In certain embodiments, the present disclosure provides multi-specific fusion proteins containing a binding region or domain specific for an IL6/IL6R complex that has one or more of the following properties: (1) greater or equal affinity for an IL6xR complex than for IL6 or IL6Rα alone or has greater affinity for IL6Rα alone or an IL6/IL6R complex than for IL6 alone, (2) competes with membrane gp130 for binding with a sIL6/IL6R complex or augments soluble gp130 binding to sIL6/IL6R complex, (3) preferentially inhibits IL6 trans-signaling over IL6 cis-signaling, or (4) does not inhibit signaling of gp130 family cytokines other than IL6. In certain preferred embodiments, a binding domain specific for an IL6/IL6R complex according to this disclosure has the following properties: (1) greater affinity for IL6Rα alone or an IL6/IL6R complex than for IL6 alone, (2) augments soluble gp130 binding to sIL6xR complex, (3) preferentially inhibits IL6 trans-signaling over IL6 cis-signaling, and (4) does not inhibit signaling of gp130 family cytokines other than IL6. For example, a binding region or domain specific for an IL6/IL6R complex may be an immunoglobulin variable binding domain or derivative thereof, such as an antibody, Fab, scFv, or the like. In the context of this disclosure, it should be understood that a binding region or domain specific for an IL6/IL6R complex is not gp130 as described herein.

As used herein, “IL6xR complex” or “IL6xR” refers to a complex of an IL6 with an IL6 receptor, wherein the IL6 receptor (also known as, for example, IL6Rα, IL6RA, IL6R1, and CD126) is either a membrane protein (referred to herein as mIL6R or mIL6Rα) or a soluble form (referred to herein as sIL6R or sIL6Rα). The term “IL6R” encompasses both mIL6Rα and sIL6Rα. In one embodiment, IL6xR comprises a complex of IL6 and mIL6Rα. In certain embodiments, the IL6xR complex is held together via one or more covalent bonds. For example, the carboxy terminus of an IL6R can be fused to the amino-terminus of an IL6 via a peptide linker, which is known in the art as a Hyper-IL6 (see, e.g., Fischer et al. (1997) Nat. Biotechnol. 15:142). A Hyper-IL6 linker can be comprised of a cross-linking compound, a one to 50 amino acid sequence, or a combination thereof. A Hyper-IL6 may further include an additional peptide tag or tags (e.g., AviFlagHis), or further include a dimerization domain, such as an immunoglobulin Fc domain or an immunoglobulin constant domain sub-region. In certain embodiments, the IL6xR complex is held together via non-covalent interactions, such as by hydrogen bonding, electrostatic interactions, Van der Waal's forces, salt bridges, hydrophobic interactions, or the like, or any combination thereof. For example, an IL6 and IL6R can naturally associate non-covalently (e.g., as found in nature, or as synthetic or recombinant proteins) or each can be fused to a domain that promotes multimerization, such as an immunoglobulin Fc domain, to further enhance complex stability.

As used herein, “gp130” refers to a signal transduction protein that binds to an IL6xR complex. The gp130 protein can be in a membrane (mgp130), soluble (sgp130), or any other functional form thereof. Exemplary gp130 proteins have a sequence as set forth in GenBank Accession No. NP_{—}002175.2 or any soluble or derivative form thereof (see, e.g., Narazaki et al. (1993) Blood 82:1120 or Diamant et al. (1997) FEBS Lett. 412:379). By way of illustration and not wishing to be bound by theory, an mgp130 protein can bind to either an IL6/mILR or an IL6/sILR complex, whereas a sgp130 primarily binds with an IL6/sILR complex (see Scheller et al. (2006) Scand. J. Immunol. 63:321). Thus, certain embodiments of binding domains, or fusion proteins thereof, of the instant disclosure can inhibit IL6xR complex trans-signaling by binding with higher affinity to IL6xR than to either IL6 or IL6Rα alone and preferably by competing with sIL6xR complex binding to mgp130. A binding domain of the instant disclosure “competes” with gp130 binding to a sIL6xR when (1) a binding domain or fusion protein thereof prevents gp130 from binding a sIL6xR and the binding domain binds sIL6xR with equal or higher affinity as compared to the binding of gp130 with sIL6xR, or (2) a binding domain or fusion protein thereof enhances or promotes sgp130 binding to sIL6xR and therby reduces the amount of time sIL6xR complex is available for binding to mgp130.

In one aspect, an IL6 antagonist binding domain of this disclosure has an affinity for IL6 or IL6xR complex that is at least 2-fold to 1000-fold greater than for IL6Rα alone or has an affinity for IL6Rα or IL6xR complex that is at least 2-fold to 1000-fold greater than for IL6 alone. By binding to IL6, IL6R, or IL6xR complex, an IL6 antagonist binding domain of this disclosure preferentially inhibits IL6 cis- and trans-signaling. In certain embodiments, the affinity of a binding domain for IL6, IL6R, or sIL6xR complex is about the same as the affinity of gp130 for IL6xR complex—with “about the same” meaning equal or up to about 2-fold higher affinity. In certain embodiments, the affinity of the binding domain for IL6, IL6R, or IL6xR complex is higher than the affinity of gp130 for IL6xR complex by at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 50-fold, at least 100-fold, 1000-fold, or greater. For example, if the affinity of gp130 for a IL6xR complex is about 2 nM (see, e.g., Gaillard et al. (1999) Eur. Cytokine Netw. 10:337), then a binding domain having at least a 10-fold higher affinity for the IL6xR complex would have a dissociation constant (K_{d}) of about 0.2 nM or less.

In further embodiments, an IL6 antagonist binding domain of this disclosure comprises a polypeptide sequence that (a) binds to a sIL6xR complex with an affinity at least 2-fold, 10-fold, 25-fold, 50-fold, 75-fold to 100-fold, 100-fold to 1000-fold higher than for either IL6 or IL6Rα alone and (b) competes with membrane gp130 for binding to sIL6xR complex or augments soluble gp130 binding to sIL6xR complex. In further embodiments, a polypeptide binding domain of this disclosure that binds to a sIL6xR complex with an affinity at least 2-fold, 10-fold, 25-fold, 50-fold, 75-fold to 100-fold, 100-fold to 1000-fold higher than for either IL6 or IL6Rα alone may also (i) more significantly or preferentially inhibit IL6 trans-signaling over IL6 cis-signaling, (ii) not inhibit signaling of gp130 cytokine family members other than IL6, (iii) preferentially inhibit IL6 trans-signaling over IL6 cis-signaling and not detectably inhibit signaling of gp130 family cytokines other than IL6, (iv) may have two or more of these properties, or (v) may have all of these properties.

In certain embodiments, a polypeptide IL6 antagonist binding domain of this disclosure binds to a sIL6xR complex with an affinity at least 2-fold to 1000-fold higher than for either IL6 or IL6Rα alone and more significantly or preferentially inhibits IL6 trans-signaling over IL6 cis-signaling. To “preferentially inhibit IL6 trans-signaling over IL6 cis-signaling” refers to altering trans-signaling to an extent that sIL6xR activity is measurably decreased while the decrease in IL6 cis-signaling is not substantially altered (i.e., meaning inhibition is minimal, non-existent, or not measurable). For example, a biomarker for sIL6xR activity (e.g., acute phase expression of antichymotrypsin (ACT) in HepG2 cells) can be measured to detect trans-signaling inhibition. A representative assay is described by Jostock et al. (Eur. J. Biochem., 2001)—briefly, HepG2 cells can be stimulated to overexpress ACT in the presence of sIL6xR (trans-signaling) or IL6 (cis-signaling), but adding spg130 will inhibit the overexpression of ACT induced by sIL6xR while not substantially affecting IL6 induced expression. Similarly, a polypeptide binding domain of this disclosure that preferentially inhibits IL6 trans-signaling over IL6 cis-signaling will inhibit the overexpression of ACT induced by sIL6xR (i.e., inhibit trans-signaling) while not substantially affecting IL6 induced expression (i.e., not measurably decrease cis-signaling). This and other assays known in the art can be used to measure preferential inhibition of IL6 trans-signaling over IL6 cis-signaling (see, e.g., other biomarkers described in Sporri et al. (1999) Int. Immunol. 11:1053; Mihara et al. (1995) Br. J. Rheum. 34:321; Chen et al. (2004) Immun. 20:59).

In further embodiments, signaling by gp130 family cytokines other than IL6 is not substantially inhibited by binding domain polypeptides or multi-specific fusion proteins thereof of this disclosure. For example, cis- and trans-signaling by an IL6xR complex via gp130 will be inhibited, but signaling by one or more other gp130 family cytokines will be minimally affected or unaffected, such as signaling via leukemia inhibitory factor (LIF), ciliary neurotropic factor (CNTF), neuropoietin (NPN), cardiotropin like cytokine (CLC), oncostatin M (OSM), IL-11, IL-27, IL-31, cardiotrophin-1 (CT-1), or any combination thereof.

It will be appreciated by those skilled in the art that the preferred in vivo half-life of a binding domain of this disclosure is on the order of days or weeks, but while the binding domain concentration may be low, the target may be plentiful as both IL6 and sIL6 production can be quite elevated in disease states (see, e.g., Lu et al. (1993) Cytokine 5:578). Thus, in certain embodiments, a binding domain of this disclosure has a k_{OFF} of about 10⁻⁵/sec (e.g., about a day) or less. In certain embodiments, the k_{OFF} can range from about 10⁻¹/sec, about 10⁻²/sec, about 10⁻³/sec, about 10⁻⁴/sec, about 10⁻⁵/sec, about 10⁻⁶/sec, about 10⁻⁷/sec, about 10⁻⁸/sec, about 10⁻⁹/sec, about 10⁻¹⁰/sec, or less.

In an illustrative example, binding domains of this disclosure specific for an 1L6 or IL6xR complex were identified in a Fab phage library of fragments (see Hoet et al. (2005) Nature Biotechnol. 23:344) by screening for binding to a synthetic IL6xR complex. The synthetic IL6xR complex used for this screening comprises a structure of N-IL6Rα(frag)-L1-IL6(frag)-L2-ID-C, wherein N is the amino-terminus and C is the carboxy-terminus, IL6Rα(frag) is a fragment of full length IL6Rα, IL6(frag) is a fragment of IL6, L1 and L2 are linkers, and ID is an intervening or dimerization domain, such as an immunoglobulin Fc domain.

More specifically, an IL6xR (which is a form of Hyper IL6) used to identify the binding domains specific for IL6xR complex has a structure, from amino-terminus to carboxy-terminus, as follows: (a) a central fragment of 212 amino acids from IL6Rα that is missing the first 110 amino acids of the full length protein and a carboxy-terminal portion that will depend on the isoform used (see GenBank Accession No. NP_{—}000556.1, isoform 1 or NP_{—}852004.1, isoform 2) fused to (2) a linker of G₃S that is in turn fused to (3) a 175 amino acid carboxy-terminal fragment of IL6 (i.e., missing the first 27 amino acids of the full length protein; GenBank Accession No. NP_{—}000591.1) that is in turn fused to (4) a linker that is an IgG2A hinge as set forth in SEQ ID NO:589, which is finally fused to a dimerization domain comprised of an immunoglobulin G1 (IgG1) Fc domain. In certain embodiments, the dimerization domain comprised of an IgG1 Fc domain has one or more of the following amino acids mutated (i.e., have a different amino acid at that position): leucine at position 234 (L234), leucine at position 235 (L235), glycine at position 237 (G237), glutamate at position 318 (E318), lysine at position 320 (K320), lysine at position 322 (K322), or any combination thereof (numbering according to EU). For example, any one of these amino acids can be substituted with alanine. In a further embodiment, an IgG1 Fc domain has each of L234, L235, G237, E318, K320, and K322 (according to EU numbering) mutated to an alanine L234A, L235A, G237A, E318A, K320A, and K322A, respectively).

In one embodiment, an IL6xR complex used to identify the IL6 antagonist binding domains of this disclosure has an amino acid sequence as set forth in SEQ ID NO:606. In certain embodiments, the IL6xR is a sIL6xR and has the amino acid sequence as set forth in SEQ ID NO:606. In further embodiments, IL6 antagonists containing a binding domain specific for an IL6xR complex (1) have greater or equal affinity for an IL6xR complex than for IL6 or IL6Rα alone, or have greater affinity for IL6Rα alone or an IL6xR complex than for IL6 alone, (2) compete with membrane gp130 for binding with a sIL6xR complex or augment soluble gp130 binding to sIL6xR complex, (3) preferentially inhibit IL6 trans-signaling over IL6 cis-signaling, or (4) do not inhibit signaling of gp130 family cytokines other than IL6, (5) have any combination thereof of properties (1)-(4), or (6) have all of the properties of (1)-(4). Other exemplary IL6xR complexes that may be used to identify binding domains of the instant disclosure or used as a reference complex to measure any of the aforementioned binding properties are described, for example, in US Patent Publication Nos. 2007/0172458; 2007/0031376; and U.S. Pat. Nos. 7,198,781; 5,919,763.

In certain embodiments, anti-IL6 binding domains comprise or are derived from the mouse monoclonal antibodies from hybridomas AH64, AH65, BSF2-77, CLB-8, CLB-12, CLB-16, HH61-08 and HH61-10, or binding fragments, or V_{H} and V_{L} domains thereof or CDRs thereof, which are all IL6 antagonist binding domains. (See e.g.,: SEQ ID NOs:787-792: AH65 & BSF2-77 light chain CDRs; SEQ ID NOs:793-798: AH65 & BSF2-77 heavy chain CDRs; SEQ ID NO: 799: AH65 light chain variable region; SEQ ID NO: 800: BSF2-77 light chain variable region; SEQ ID NO: 801: humanized AH 65-1 light chain variable region; SEQ ID NO: 802: humanized AH65-3 light chain variable region; SEQ ID NO: 803: humanized BSF2-77-1 light chain variable region; SEQ ID NO: 804: humanized BSF2-77-2 light chain variable region; SEQ ID NO: 805: AH65 heavy chain variable region; SEQ ID NO: 806: BSF2-77 heavy chain variable region; SEQ ID NO: 807: humanized AH 65-1 heavy chain variable region; SEQ ID NO: 808: humanized AH65-3 heavy chain variable region; SEQ ID NO: 809: humanized BSF2-77-1 heavy chain variable region; SEQ ID NO: 810: humanized BSF2-77-2 heavy chain variable region; SEQ ID NO: 811-822: DNAs encoding for SEQ ID NO: 799-810).

In some embodiments, IL6 antagonist binding domains of this disclosure comprise V_{H} and V_{L} domains specific for an IL6, IL6R, or IL6xR complex as described herein, and preferably human IL6, human IL6R, or human IL6xR complex. In certain embodiments, the V_{H} and V_{L} domains are rodent (e.g., mouse, rat), humanized, or human. Examples of binding domains containing such V_{H} and V_{L} domains specific for IL6, IL6R, or IL6xR are set forth in SEQ ID NOS:435-496 and 373-434, respectively. In further embodiments, there are provided polypeptide binding domains specific for an IL6xR complex that bind to the IL6xR with a higher or equal affinity than either IL6 or IL6Rα alone, and either compete with membrane gp130 for binding to the sIL6xR complex or augment soluble gp130 binding to sIL6xR complex, wherein the binding domain comprises a sequence that is at least 90%, at least 91%, at least 92%, at least 93%, at least 94%, at least 95%, at least 96%, at least 97%, at least 98%, at least 99%, at least 99.5% , or at least 100% identical to an amino acid sequence of one or more light chain variable regions (V_{L}) or to one or more heavy chain variable regions (V_{H}), or both, as set forth in SEQ ID NOS:373-434 and 435-496, respectively, wherein each CDR has up to three amino acid substitutions (for instance, the substitutions may be in the framework regions).

In further embodiments, binding domains of this disclosure comprise V_{H} and V_{L} domains specific for an IL6xR as set forth in SEQ ID NOS:435-496 and 373-434, respectively, which are at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of such V_{H} domain, V_{L} domain, or both, wherein each CDR has at most from zero to three amino acid substitutions. For example, the amino acid sequence of a V_{H} domain, V_{L} domain, or both of this disclosure can be at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of V_{H} domain (e.g., amino acids 512 to 631), V_{L} domain (e.g., amino acids 649 to 758), or both, respectively, from a multispecific fusion protein containing binding domain TRU(XT6)-1002 (SEQ ID NO:608), wherein each CDR has at most from zero to three amino acid substitutions.

In any of these or other embodiments described herein, the V_{L} and V_{H} domains may be arranged in either orientation and may be separated by five up to about a thirty-five amino acid linker as disclosed herein or any other amino acid sequence capable of providing a spacer function such that the two sub-binding domains can interact to form a functional binding domain. In certain embodiments, a linker joining the V_{H} and V_{L} domains (also referred to herein as a “variable domain linker”) includes those belonging to the (GlyₙSer) family, such as (Gly₃Ser)ₙ(Gly₄Ser)₁, (Gly₃Ser)₁(Gly₄Ser)ₙ, (Gly₃Ser)ₙ(Gly₄Ser)ₙ, or (Gly₄Ser)ₙ, wherein n is an integer of 1 to 6. In certain embodiments, the linker is GGGGSGGGGSGGGGS (SEQ ID NO:542). In preferred embodiments, these (GlyₙSer)-based linkers are used to link the VH and VL domains in a binding domain, but are not used to link a binding domain to any other domain, e.g., a heterodimerization domain or to an Fc region portion. In some embodiments, the linker joining the V_{H} and V_{L} domains comprises an amino acid sequence as set forth in SEQ ID NO:497-604, 823-828, 830-845, and 851-1106, such as Linker 46 (SEQ ID NO:542), Linker 47 (SEQ ID NO:543) or Linker 80 (SEQ ID NO:576).

In further embodiments, IL6 antagonist binding domains of this disclosure may comprise one or more complementarity determining region (“CDR”), or multiple copies of one or more such CDRs, which have been obtained, derived, or designed from variable regions of an anti-IL6, anti-IL6R, or anti-IL6xR complex scFv or Fab fragment or from heavy or light chain variable regions thereof. Thus, a binding domain of this disclosure can comprise a single CDR3 from a variable region of an anti-IL6, anti-IL6R, or anti-IL6xR, or it can comprise multiple CDRs that can be the same or different. In certain embodiments, IL6 antagonist binding domains of this disclosure comprise V_{H} and V_{L} domains comprising framework regions and CDR1, CDR2 and CDR3 regions, wherein (a) the V_{H} domain comprises the amino acid sequence of a heavy chain CDR3 found in any one of SEQ ID NOS:435-496; or (b) the V_{L} domain comprises the amino acid sequence of a light chain CDR3 found in any one of SEQ ID NOS:373-434; or (c) the binding domain comprises a V_{H} amino acid sequence of (a) and a V_{L} amino acid sequence of (b); or the binding domain comprises a V_{H} amino acid sequence of (a) and a V_{L} amino acid sequence of (b) and wherein the V_{H} and V_{L} are found in the same reference sequence. In further embodiments, binding domains of this disclosure comprise V_{H} and V_{L} domains specific for an IL6xR complex comprising framework regions and CDR1, CDR2 and CDR3 regions, wherein (a) the V_{H} domain comprises the amino acid sequence of a heavy chain CDR1, CDR2, and CDR3 found in any one of SEQ ID NOS:435-496; or (b) the V_{L} domain comprises the amino acid sequence of a light chain CDR1, CDR2, and CDR3 found in any one of SEQ ID NOS:373-434; or (c) the binding domain comprises a V_{H} amino acid sequence of (a) and a V_{L} amino acid sequence of (b); or the binding domain comprises a V_{H} amino acid sequence of (a) and a V_{L} amino acid sequence of (b), wherein the V_{H} and V_{L} amino acid sequences are from the same reference sequence. Exemplary light and heavy chain variable domain CDRs directed against IL6, IL6R, or IL6xR are provided in SEQ ID NOS:1-187 and 787-792, and SEQ ID NOS:187-372 and 793-798, respectively.

Amino acid sequences of IL6 antagonist light chain variable regions are provided in SEQ ID NOS:373-434 and 799-804 and IL6 antagonist heavy chain variable regions are provided in SEQ ID NO:435-496 and 805-810.

In any of the embodiments described herein comprising specific CDRs against IL6, IL6R, or IL6xR, a binding domain can comprise (i) a V_{H} domain having an amino acid sequence that is at least 80%, 85%, 90%, 91%, 92%, 93%, 94%, 95%, 96%, 97%, 98%, or 99% identical to the amino acid sequence of a V_{H} domain found in any one of SEQ ID NOS:435-496 and 805-810; or (ii) a V_{L} domain having an amino acid sequence that is at least 80%, 85%, 90%, 91%, 92%, 93%, 94%, 95%, 96%, 97%, 98%, or 99% identical to the amino acid sequence of a V_{L} domain found in any one of SEQ ID NOS:373-434 and 799-804; or (iii) both a V_{H} domain of (i) and a V_{L} domain of (ii); or both a V_{H} domain of (i) and a V_{L} domain of (ii) wherein the V_{H} and V_{L} are from the same reference sequence.

In certain embodiments, a binding domain of this disclosure may be an immunoglobulin-like domain, such as an immunoglobulin scaffold. Immunoglobulin scaffolds contemplated in this disclosure include a scFv, Fab, a domain antibody, or a heavy chain-only antibody. In further embodiments, there are provided anti-IL6 or anti-IL6xR antibodies (e.g., non-human such as mouse or rat, chimeric, humanized, human) or Fab fragments or scFv fragments that have an amino acid sequence that is at least 80%, 85%, 90%, 91%, 92%, 93%, 94%, 95%, 96%, 97%, 98%, 99%, or 100% identical to the amino acid sequence of a V_{H} and V_{L} domain set in any one of SEQ ID NOS:435-496 and 805-801, and 373-434 and 799-804, respectively, which may also have one or more of the following properties: (1) have greater or equal affinity for an IL6xR complex than for IL6 or IL6Rα alone, or have greater affinity for IL6Rα alone or an IL6xR complex than for IL6 alone, (2) compete with membrane gp130 for binding with a sIL6xR complex or augment soluble gp130 binding to sIL6xR complex, (3) preferentially inhibit IL6 trans-signaling over IL6 cis-signaling, or (4) do not inhibit signaling of gp130 family cytokines other than IL6. Such antibodies, Fabs, or scFvs can be used in any of the methods described herein. In certain embodiments, the present disclosure provides polypeptides containing a binding domain that is an IL6 antagonist (i.e., can inhibit IL6 cis- and trans-signaling). In further embodiments, an IL6 antagonist according to this disclosure does not inhibit signaling of gp130 family cytokines other than IL6. Exemplary IL6 antagonists include binding domains specific for an IL6 or IL6xR, such as an immunoglobulin variable binding domain or derivative thereof (e.g., an antibody, Fab, scFv, or the like).

Alternatively, binding domains of this disclosure may be part of a scaffold other than an immunoglobulin. Other scaffolds contemplated include an A domain molecule, a fibronectin III domain, an anticalin, an ankyrin-repeat engineered binding molecule, an adnectin, a Kunitz domain, or a protein AZ domain affibody.

### Making TNF and IL6 Antagonists

A leader peptide may be used to facilitate secretion of expressed IL6 antagonist and TNF-α antagonist polypeptides and fusion proteins. Using any of the conventional leader peptides (signal sequences) can be expected to direct nascently expressed polypeptides or fusion proteins into a secretory pathway and to result in cleavage of the leader peptide from the mature polypeptide or fusion protein at or near the junction between the leader peptide and the polypeptide or fusion protein. A particular leader peptide will be chosen based on considerations known in the art, such as using sequences encoded by polynucleotides that allow the easy inclusion of restriction endonuclease cleavage sites at the beginning or end of the coding sequence for the leader peptide to facilitate molecular engineering, provided that such introduced sequences specify amino acids that either do not interfere unacceptably with any desired processing of the leader peptide from the nascently expressed protein or do not interfere unacceptably with any desired function of a polypeptide or fusion protein molecule if the leader peptide is not cleaved during maturation of the polypeptides or fusion proteins. Exemplary leader peptides of this disclosure include natural leader sequences or others, such as H₃N-MDFQVQIFSFLLISASVIMSRG(X)ₙ—CO₂H, wherein X is any amino acid and n is zero to three (SEQ ID NOS:785, 848-850) or H₃N-MEAPAQLLFLLLLWLPDTTG-CO₂H (SEQ ID NO:786).

In certain embodiments of the invention, the IL6 antagonist and/or TNF-α antagonist is glycosylated, the pattern of glycosylation being dependent upon a variety of factors including the host cell in which the protein is expressed (if prepared in recombinant host cells) and the culture conditions. Glycosylation patterns may be modified using the methods disclosed in US 2009/0041765 and US 2010/0150948, each of which is herein incorporated by reference in its entirety.

This disclosure also provides derivatives of the binding domain of this disclosure or of fusion proteins comprising such binding domain.

Derivatives include specific binding agent polypeptides bearing modifications other than insertion, deletion, or substitution of amino acid residues. Preferably, the modifications are covalent in nature, and include for example, chemical bonding with polymers, lipids, other organic, and inorganic moieties. Derivatives of this disclosure may be prepared to increase circulating half-life of a specific binding agent polypeptide, or may be designed to improve targeting capacity for the polypeptide to desired cells, tissues, or organs.

In certain embodiments, the in vivo half-life of the binding domain polypeptide or fusion protein thereof of this disclosure can be increased using methods known in the art for increasing the half-life of large molecules. For example, this disclosure embraces fusion proteins that are covalently modified or derivatized to include one or more water-soluble polymer attachments, such as polyethylene glycol, polyoxyethylene glycol, or polypropylene glycol (see, e.g., U.S. Pat. Nos. 4,640,835; 4,496,689; 4,301,144; 4,670,417; 4,791,192; 4,179,337). Still other useful polymers known in the art include monomethoxy-polyethylene glycol, dextran, cellulose, and other carbohydrate-based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of these polymers. Particularly preferred are polyethylene glycol (PEG)-derivatized proteins. Water-soluble polymers may be bonded at specific positions, for example at the amino terminus of the proteins and polypeptides according to this disclosure, or randomly attached to one or more side chains of the polypeptide. The use of PEG for improving therapeutic capacities is described in U.S. Pat. No. 6,133,426.

Such methods also include creating fusion proteins wherein the binding domain is fused to a protein that conveys a longer half life to the binding domain fusion protein than that of the binding domain alone. Such fusion proteins can include proteins that themselves bind to proteins that have a long half life, e.g., immunoglobulin, immunoglobulin Fc domains, transferrin, streptococcal G protein, or albumin. Such fusions of binding domains to stable plasma proteins are disclosed, e.g., in U.S. Pat. Nos. 5,428,130; 5,116,964.

A particular embodiment of this disclosure is an immunoglobulin or an Fc fusion protein. Such a fusion protein can have a long half-life, e.g., several hours, a day or more, or even a week or more, especially if the Fc domain is capable of interacting with FcRn, the neonatal Fc receptor. The binding site for FcRn in an Fc domain is also the site at which the bacterial proteins A and G bind. The tight binding between these proteins can be used as a means to purify antibodies or fusion proteins of this disclosure by, for example, employing protein A or protein G affinity chromatography during protein purification.

As indicated herein, antagonist fusion polypeptides of the present disclosure may comprise an Fc region constant domain portion (also referred to as an Fc region portion). The inclusion of an Fc region portion slows clearance of the heterodimers from circulation after administration to a subject. By mutations or other alterations, the Fc region portion further enables relatively easy modulation of heterodimer polypeptide effector functions (e.g., ADCC, ADCP, CDC, complement fixation and binding to Fc receptors), which can either be increased or decreased depending on the disease being treated, as known in the art and described herein. In certain embodiments, an Fc region portion will be capable of mediating one or more of these effector functions.

An Fc region portion for use in the fusion proteins of the present disclosure may comprise a CH2 domain, a CH3 domain, a CH4 domain or any combination thereof. For example, an Fc region portion may comprise a CH2 domain, a CH3 domain, both CH2 and CH3 domains, both CH3 and CH4 domains, two CH3 domains, a CH4 domain, or two CH4 domains. In certain embodiments, a CH2, CH3 and/or CH4 domain that may form an Fc region portion of an antagonist binding protein of the present disclosure may be a wild type immunoglobulin CH2, CH3, or CH4 domain or an altered immunoglobulin CH2, CH3, or CH4 domain thereof from certain immunoglobulin classes or subclasses (e.g., IgG1, IgG2, IgG3, IgG4, IgA1, IgA2, or IgD) and from various species (including human, mouse, rat, and other mammals). Illustrative altered domains with desired functional attributes are described, for example, in PCT WO2010/042904, incorporated herein in its entirety.

Protein purification techniques are well known to those of skill in the art. These techniques involve, at one level, the crude fractionation of the polypeptide and non-polypeptide fractions. Further purification using chromatographic and electrophoretic techniques to achieve partial or complete purification (or purification to homogeneity) is frequently desired. Analytical methods particularly suited to the preparation of a pure fusion protein are ion-exchange chromatography, exclusion chromatography; polyacrylamide gel electrophoresis; and isoelectric focusing. Particularly efficient methods of purifying peptides are fast protein liquid chromatography and HPLC.

Certain aspects of the present disclosure concern the purification, and in particular embodiments, the substantial purification, of a polypeptide of this disclosure. The term “purified” as used herein, is intended to refer to a composition, isolatable from other components, wherein the fusion protein is purified to any degree relative to its naturally obtainable state. A purified protein therefore also refers to such protein, isolated from the environment in which it naturally occurs.

Generally, “purified” will refer to a polypeptide composition that has been subjected to fractionation to remove various other components, and which composition substantially retains its expressed biological activity. Where the term “substantially purified” is used, this designation refers to a binding domain protein composition in which the protein forms the major component of the composition, such as constituting about 50%, about 60%, about 70%, about 80%, about 90%, about 95%, about 99% or more of the protein, by weight, in the composition.

Various methods for quantifying the degree of purification are known to those of skill in the art in light of the present disclosure. These include, for example, determining the specific binding activity of an active fraction, or assessing the amount of protein in a fraction by SDS/PAGE analysis. A preferred method for assessing the purity of a protein fraction is to calculate the binding activity of the fraction, to compare it to the binding activity of the initial extract, and to thus calculate the degree of purification, herein assessed by a “-fold purification number.” The actual units used to represent the amount of binding activity will, of course, be dependent upon the particular assay technique chosen to follow the purification and whether or not the expressed protein exhibits a detectable binding activity.

Various techniques suitable for use in protein purification are well known to those of skill in the art. These include, for example, precipitation with ammonium sulfate, PEG, antibodies and the like, or by heat denaturation, followed by centrifugation; chromatography steps such as ion exchange, gel filtration, reverse phase, hydroxylapatite and affinity chromatography; isoelectric focusing; gel electrophoresis; and combinations of these and other techniques. As is generally known in the art, the order of conducting various purification steps may be changed, or certain steps may be omitted and still result in a suitable method for the preparation of a substantially purified protein.

### Linkers

The fusion proteins of this disclosure may comprise one or more peptide linkers between the various domains as described herein. In addition to providing a spacing function, a linker can provide flexibility or rigidity suitable for properly orienting the one or more binding domains of a fusion protein, both within the fusion protein and between or among the fusion proteins and their target(s). Further, a linker can support expression of a full-length fusion protein and stability of the purified protein both in vitro and in vivo following administration to a subject in need thereof, such as a human, and is preferably non-immunogenic or poorly immunogenic in those same subjects. In certain embodiments, a linker of a dimerization domain of fusion proteins of this disclosure may comprise part or all of a human immunoglobulin hinge.

Exemplary linkers that can be used to join an intervening domain (e.g., an immunoglobulin-derived constant sub-region) to a binding domain or to join two variable regions of a binding domain are set forth in SEQ ID NOS:497-604, 823-828 and 830-845. Illustrative linkers that may be used in constructing the antagonists described herein also include SEQ ID NOs:851-1106.

Linkers contemplated in this disclosure include, for example, peptides derived from any interdomain region of an immunoglobulin superfamily member (e.g., an antibody hinge region) or a stalk region of C-type lectins, a family of type II membrane proteins. These linkers range in length from about two to about 150 amino acids, or about two to about 40 amino acids, or about eight to about 20 amino acids preferably about ten to about 60 amino acids, more preferably about 10 to about 30 amino acids, and most preferably about 15 to about 25 amino acids. For example, Linker 1 (SEQ ID NO:497) is two amino acids in length and Linker 116 (SEQ ID NO:603) is 36 amino acids in length.

Beyond general length considerations, a linker suitable for use in the fusion proteins of this disclosure includes an antibody hinge region selected from an IgG hinge, IgA hinge, IgD hinge, IgE hinge, or variants thereof. In certain embodiments, a linker may be an antibody hinge region (upper and core region) selected from human IgG1, human IgG2, human IgG3, human IgG4, or fragments or variants thereof. As used herein, a linker that is an “immunoglobulin hinge region” refers to the amino acids found between the carboxyl end of CH1 and the amino terminal end of CH2 (for IgG, IgA, and IgD) or the amino terminal end of CH3 (for IgE and IgM). A “wild type immunoglobulin hinge region,” as used herein, refers to a naturally occurring amino acid sequence interposed between and connecting the CH1 and CH2 regions (for IgG, IgA, and IgD) or interposed between and connecting the CH2 and CH3 regions (for IgE and IgM) found in the heavy chain of an antibody. In preferred embodiments, the wild type immunoglobulin hinge region sequences are human.

Accordingly, in certain embodiments, a hinge is a wild type human immunoglobulin hinge region (e.g., human immunoglobulin hinge regions as set forth in SEQ ID NOs:851-857). Exemplary human wild type immunoglobulin hinge regions are set forth in SEQ ID NOS:851 (IgA1 hinge), 852 (IgA2 hinge), 853 (IgD hinge), 854 (IgG1 hinge), 855 (IgG2 hinge), 856 (IgG3 hinge) and 857 (IgG4 hinge). In certain other embodiments, one or more amino acid residues may be added at the amino- or carboxy- terminus of a wild type immunoglobulin hinge region as part of a fusion protein construct design. For example, additional junction amino acid residues at the hinge amino-terminus can be “RT,” “RSS,” “TG,” or “T”, or at the hinge carboxy-terminus can be “SG”, or a hinge deletion can be combined with an addition, such as ΔP with “SG” added at the carboxyl terminus.

According to crystallographic studies, an IgG hinge domain can be functionally and structurally subdivided into three regions: the upper hinge region, the core or middle hinge region, and the lower hinge region (Shin et al. (1992) Immunol. Rev. 130:87). Exemplary upper hinge regions include EPKSCDKTHT (SEQ ID NO:830) as found in IgG1, ERKCCVE (SEQ ID NO:831) as found in IgG2, ELKTPLGDTT HT (SEQ ID NO:832) or EPKSCDTPPP (SEQ ID NO:833) as found in IgG3, and ESKYGPP (SEQ ID NO:834) as found in IgG4. Exemplary middle hinge regions include CPPCP (SEQ ID NO:835) as found in IgG1 and IgG2, CPRCP (SEQ ID NO:836) as found in IgG3, and CPSCP (SEQ ID NO:837) as found in IgG4. While IgG1, IgG2, and IgG4 antibodies each appear to have a single upper and middle hinge, IgG3 has four in tandem—one of ELKTPLGDTT HTCPRCP (SEQ ID NO:838) and three of EPKSCDTPPP CPRCP (SEQ ID NO:839).

IgA and IgD antibodies appear to lack an IgG-like core region, and IgD appears to have two upper hinge regions in tandem (see SEQ ID NOS:840 and 841). Exemplary wild type upper hinge regions found in IgA1 and IgA2 antibodies are set forth in SEQ ID NOS:842 and 843.

IgE and IgM antibodies, in contrast, instead of a typical hinge region have a CH2 region with hinge-like properties. Exemplary wild-type CH2 upper hinge-like sequences of IgE and IgM are set forth in SEQ ID NO:844 (VCSRDFTPPT VKILQSSSDG GGHFPPTIQL LCLVSGYTPG TINITWLEDG QVMDVDLSTA STTQEGELAS TQSELTLSQK HWLSDRTYTC QVTYQGHTFE DSTKKCA) and SEQ ID NO:845 (VIAELPPKVS VFVPPRDGFF GNPRKSKLIC QATGFSPRQI QVSWLREGKQ VGSGVTTDQV QAEAKESGPT TYKVTSTLTI KESDWLGQSM FTCRVDHRGL TFQQNASSMC VP), respectively.

In certain embodiments, a hinge is an altered immunoglobulin hinge in which one or more cysteine residues in a wild type immunoglobulin hinge region is substituted with one or more other amino acid residues (e.g., serine or alanine). For example, a hinge may be an altered immunoglobulin hinge based on or derived from a wild type human IgG1 hinge as set forth in SEQ ID NO:854, which from amino terminus to carboxyl terminus comprises the upper hinge region (EPKSCDKTHT, SEQ ID NO:830) and the core hinge region (CPPCP, SEQ ID NO:835). Exemplary altered immunoglobulin hinges include an immunoglobulin human IgG1 hinge region having one, two or three cysteine residues found in a wild type human IgG1 hinge substituted by one, two or three different amino acid residues (e.g., serine or alanine). An altered immunoglobulin hinge may additionally have a proline substituted with another amino acid (e.g., serine or alanine). For example, the above-described altered human IgG1 hinge may additionally have a proline located carboxyl terminal to the three cysteines of wild type human IgG1 hinge region substituted by another amino acid residue (e.g., serine, alanine). Preferably the prolines of the core hinge region are not substituted. Exemplary altered immunoglobulin hinges are set forth in SEQ ID NOS: 865-903. A preferred altered IgG1 hinge is an altered human IgG1 hinge in which the first cysteine is substituted by serine. The sequence of this preferred altered IgG1 hinge is set forth in SEQ ID NO:878, and is referred to as the “ human IgG1 SCC-P hinge” or “SCC-P hinge.” In certain embodiments, one or more amino acid residues (e.g., “RT,” “RSS,” or “T”) may be added at the amino-or carboxy-terminus of a mutated immunoglobulin hinge region as part of a fusion protein construct design.

In certain embodiments, a hinge polypeptide comprises or is a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a wild type immunoglobulin hinge region, such as a wild type human IgG1 hinge, a wild type human IgG2 hinge, or a wild type human IgG4 hinge.

Alternative hinge and linker sequences that can be used as connecting regions may be crafted from portions of cell surface receptors that connect IgV-like or IgC-like domains. Regions between IgV-like domains where the cell surface receptor contains multiple IgV-like domains in tandem and between IgC-like domains where the cell surface receptor contains multiple tandem IgC-like regions could also be used as connecting regions or linker peptides. In certain embodiments, hinge and linker sequences are from 5 to 60 amino acids long, and may be primarily flexible, but may also provide more rigid characteristics, may contain primarily a helical structure with minimal (3 sheet structure. Preferably, sequences are stable in plasma and serum and are resistant to proteolytic cleavage. In some embodiments, sequences may contain a naturally occurring or added motif such as CPPCP (SEQ ID NO:835) that confers the capacity to form a disulfide bond or multiple disulfide bonds to stabilize the C-terminus of the molecule. In other embodiments, sequences may contain one or more glycosylation sites. Examples of hinge and linker sequences include interdomain regions between IgV-like and IgC-like or between IgC-like or IgV-like domains of CD2, CD4, CD22, CD33, CD48, CD58, CD66, CD80, CD86, CD96, CD150, CD166, and CD244. Alternative hinges may also be crafted from disulfide-containing regions of Type II receptors from non-immunoglobulin superfamily members, such as CD69, CD72, and CD161.

In some embodiments, a hinge linker has a single cysteine residue for formation of an interchain disulfide bond. In other embodiments, a linker has two cysteine residues for formation of interchain disulfide bonds. In further embodiments, a hinge linker is derived from an immunoglobulin interdomain region (e.g., an antibody hinge region) or a Type II C-type lectin stalk region (derived from a Type II membrane protein; see, e.g., exemplary lectin stalk region sequences set forth in of PCT Application Publication No. WO 2007/146968, such as SEQ ID NOS:111, 113, 115, 117, 119, 121, 123, 125, 127, 129, 131, 133, 135, 149, 151, 153, 155, 157, 159, 161, 163, 165, 167, 169, 231, 233, 235, 237, 239, 241, 243, 245, 247, 249, 251, 253, 255, 257, 259, 261, 263, 265, 267, 269, 271, 273, 275, 277, 279, 281, 287, 289, 297, 305, 307, 309-311, 313-331, 346, 373-377, 380, or 381 from that publication), which sequences are hereby incorporated by reference.

In further embodiments, a hinge present in a single chain polypeptide may be a hinge that is not based on or derived from an immunoglobulin hinge (i.e., not a wild type immunoglobulin hinge or an altered immunoglobulin hinge). In certain embodiments, these types of non-immunoglobulin based hinges are used on or near the carboxyl end (e.g., located carboxyl terminal to Fc region portions) of the antagonist binding fusion proteins disclosed herein. Examples for such hinges include peptides of about five to about 150 amino acids of the interdomain or stalk region of type II C-lectins or CD molecules, preferably having about eight to 25 amino acids, more preferably having seven to 18 amino acids, and derivatives thereof.

The “interdomain or stalk region” of a type II C-lectin or CD molecule refers to the portion of the extracellular domain of the type II C-lectin or CD molecule that is located between the C-type lectin-like domain (CTLD; e.g., similar to CTLD of natural killer cell receptors) and the transmembrane domain. For example, in the human CD94 molecule (GenBank Accession No. AAC50291.1, PRI Nov. 30, 1995), the extracellular domain corresponds to amino acid residues 34-179, whereas the CTLD corresponds to amino acid residues 61-176. Accordingly, the interdomain or stalk region of the human CD94 molecule includes amino acid residues 34-60, which is found between the membrane and the CTLD (see Boyington et al., Immunity 10:75, 1999; for descriptions of other stalk regions, see also Beavil et al., Proc. Nat'l. Acad. Sci. USA 89:753, 1992; and Figdor et al., Nature Rev. Immunol. 2:77, 2002). These type II C-lectin or CD molecules may also have from six to 10 junction amino acids between the stalk region and the transmembrane region or the CTLD. In another example, the 233 amino acid human NKG2A protein (GenBank Accession No. P26715.1, PRI Jun. 15, 2010) has a transmembrane domain ranging from amino acids 71-93 and an extracellular domain ranging from amino acids 94-233. The CTLD is comprised of amino acids 119-231, and the stalk region comprises amino acids 99-116, which is flanked by junctions of five and two amino acids. Other type II C-lectin or CD molecules, as well as their extracellular ligand-bind domains, interdomain or stalk regions, and CTLDs are known in the art (see, e.g., GenBank Accession Nos. NP_{—}001993.2; AAH07037.1, PRI Jul. 15, 2006; NP_{—}001773.1, PRI Jun. 20, 1010; AAL65234.1, PRI Jan. 17, 2002, and CAA04925.1, PRI Nov. 14, 2006, for the sequences of human CD23, CD69, CD72, NKG2A and NKG2D and their descriptions, respectively).

A “derivative” of an interdomain or stalk region, or fragment thereof, of a type II C-lectin or CD molecule refers to an about an eight to about 150 amino acid sequence in which one, two, or three amino acids of the stalk region of a wild type type II C-lectin or CD molecule have a deletion, insertion, substitution, or any combination thereof, preferably the one or more changes are substitutions or the one or more mutations include only one deletion. In further embodiments, a derivative of an interdomain or stalk region is more resistant to proteolytic cleavage as compared to the wild-type interdomain or stalk region sequence, such as those derived from about eight to about 20 amino acids of NKG2A, NKG2D, CD23, CD64, CD72, or CD94.

In certain embodiments, interdomain or stalk region hinges have seven to 18 amino acids and can form an a-helical coiled coil structure. In certain embodiments, interdomain or stalk region hinges contain 0, 1, 2, 3, or 4 cysteines. Exemplary interdomain or stalk region hinges are peptide fragments of the interdomain or stalk regions, such as ten to 150 amino acid fragments from the stalk regions of CD69, CD72, CD94, NKG2A and NKG2D, as set forth in SEQ ID NOS:858-864. Additional exemplary stalk region or interdomain hinges useful in the fusion proteins of this disclosure include those as set forth in SEQ ID NOS:990-999, 907-931, and 933.

In certain embodiments, hinge sequences have 5 to 150 amino acids, 5 to 10 amino acids, 10 to 20 amino acids, 20 to 30 amino acids, 30 to 40 amino acids, 40 to 50 amino acids, 50 to 60 amino acids, 5 to 60 amino acids, 5 to 40 amino acids, preferably have 8 to 20, more preferably have 10 to 15 amino acids, and may be primarily flexible, but may also provide more rigid characteristics or may contain primarily α-helical structure with minimal β-sheet structure. The lengths or the sequences of the hinges may affect the binding affinities of the binding domains to which the hinges are directly or indirectly (via another region or domain) connected as well as one or more activities of the Fc region portions to which the hinges are directly or indirectly connected.

Preferably, hinge sequences are stable in plasma and serum and are resistant to proteolytic cleavage. In certain embodiments, the first lysine in the IgG1 upper hinge region is mutated to minimize proteolytic cleavage, preferably the lysine is substituted with methionine, threonine, alanine or glycine, or is deleted (see, e.g., SEQ ID NOS:934-989, which may include junction amino acids at the amino terminus, preferably RT). Additional exemplary hinges are set forth in SEQ ID NOS:1000-1106.

In certain embodiments where a single chain polypeptide comprises a binding domain at or near its carboxyl terminus, a hinge may be present to link the binding domain with another portion of the single chain polypeptide (e.g., an Fc region portion or other domain). Preferably, such a hinge is a non-immunoglobulin hinge (i.e., a hinge not based on or derived from a wild type immunoglobulin hinge) and may be a stalk region of a type II C-lectin or CD molecule, an interdomain region that connect IgV-like or IgC-like domains of a cell surface receptor, or a derivative or functional variant thereof. Exemplary carboxyl terminal hinges, sometimes referred to as “back-end” hinges, includes those set forth in SEQ ID NOS:990-999, 907-931, and 933.

In one aspect, fusion proteins of this disclosure comprise a TNF-α antagonist as described elsewhere herein (TNF-α binding domain), IL6, IL6R, or IL6xR, in the form of a SMIP protein. Methods for making SMIP proteins are described herein and are known in the art (see U.S. Patent Publication Nos. 2003/0133939, 2003/0118592, and 2005/0136049). In certain embodiments, a fusion protein has a polypeptide binding domain specific for TNF-α or for an IL6xR complex that binds to the IL6xR with a higher affinity than either IL6 or IL6Rα alone, and competes with gp130 for binding to the sIL6xR complex or enhances gp130 binding with sIL6xR, wherein the fusion protein comprises from amino-terminus to carboxy-terminus, (a) the polypeptide binding domain, fused to (b) a first linker comprising a hinge region polypeptide, fused to (b) an immunoglobulin heavy chain CH2 constant region or sub-region polypeptide, fused to (c) an immunoglobulin heavy chain CH3 constant region or sub-region polypeptide. Alternatively, a SMIP protein structure can be illustrated as follows: N-BD-L1-CH2CH3-C, wherein N is the amino-terminus of the fusion protein, BD is the anti-IL6xR complex binding domain or scFv, L1 is a linker, CH2 and CH3 are immunoglobulin constant heavy regions 2 and 3, and C is the carboxy-terminus of the fusion protein. In some embodiments, the linker is a (Gly₄Ser)ₙ wherein n is an integer of 1 to 6, such as Linker 46 (SEQ ID NO:542), or the linker is an IgG1, IgA or IgE hinge region, a mutant IgG1 hinge region having zero, one, or two cysteine residues, such as Linker 47 (SEQ ID NO:543), or Linker 80 (SEQ ID NO:576). In some embodiments, the fusion protein will be fused, via linker or not, to a domain other than an immunoglobulin constant region or sub-region so that the fusion protein remains primarily or substantially a single chain polypeptide in a composition.

In further embodiments, a SMIP fusion protein of this disclosure has a binding domain that comprises a light chain variable region containing CDR1, CDR2, and CDR3 sequences that are each at least 80% to 100% identical to at least one light chain variable region CDR1, CDR2, and CDR3 as set forth in any one of SEQ ID NOS:373-434 and 799-804, respectively, wherein each CDR has from zero to three amino acid changes, and comprises a heavy chain variable region containing CDR1, CDR2, and CDR3 sequences that are each at least 80% to 100% identical to at least one heavy chain variable region CDR1, CDR2, and CDR3 as set forth in any one of SEQ ID NOS:435-496 and 805-810, respectively, wherein each CDR has from zero to three amino acid changes. In still further embodiments, a SMIP fusion protein of this disclosure has an amino acid sequence at least 80%, 85%, 90%, 91%, 92%, 93%, 94%, 95%, 96%, 97%, 98%, 99%, or 100% identical to an amino acid sequence set forth in any one of SEQ ID NOS:671-694, with or without a leader peptide sequence.

In still other embodiments, SMIP polypeptides can have a binding region or domain that is an IL6 antagonist, wherein IL6 cis- and trans-signaling is measurably inhibited. In certain embodiments, an IL6 antagonist according to this disclosure does not inhibit signaling of gp130 family cytokines other than IL6.

In further embodiments, fusion proteins of this disclosure comprise an IL6 antagonist binding domain in the form of a PIMS protein wherein the binding domain is disposed at the carboxy-terminus of the fusion protein. Constructs and methods for making PIMS proteins are described in PCT Publication No. WO 2009/023386. In general, a PIMS molecule is a single-chain polypeptide comprising, in amino-terminal to carboxy-terminal orientation, an intervening domain (e.g., an immunoglobulin constant sub-region derived from that includes a CH2 and CH3 domain from the same (preferred) or different animal species, immunoglobulin isotype and/or immunoglobulin sub-class), a linker peptide (e.g., an immunoglobulin hinge region or stalk region of type II C-lectin), and a specific binding domain. In some embodiments, a PIMS molecule further contains an amino-terminally disposed immunoglobulin hinge region, and the amino-terminal hinge region may be the same as, or different than, the linker found between the dimerization domain and the binding domain. In some embodiments, an amino-terminally disposed linker contains a naturally occurring or added motif (such as CPPC) to promote the formation of at least one disulfide bond to stabilize the amino-terminus of a multimerized molecule. Thus, exemplary schematic organizations of some PIMS molecules include N-dimerization domain-linker-binding domain-C or N-hinge linker-dimerization domain-linker-binding domain-C. In some embodiments, the fusion protein will have an intervening domain wherein the fusion protein remains primarily or substantially as a single chain polypeptide in a composition or is found primarily or substantially as a dimer in a composition.

In certain embodiments, a fusion protein has a IL6 antagonist polypeptide binding domain that binds an IL6xR complex with a higher affinity than either IL6 or IL6Rα alone, and competes with gp130 for binding to the sIL6xR complex or enhances gp130 binding to sIL6xR complex, wherein, from carboxy-terminus to amino-terminus, (a) the polypeptide binding domain is fused to a first linker, (b) the first linker is fused to an immunoglobulin heavy chain CH3 constant region or sub-region polypeptide, and (c) the CH3 constant region or sub-region polypeptide is fused to an immunoglobulin heavy chain

CH2 constant region or sub-region polypeptide, and (d) the CH2 constant region or sub-region polypeptide is fused to a second linker. In further embodiments, a PIMS fusion protein of this disclosure has a binding domain that comprises a light chain variable region containing CDR1, CDR2, and CDR3 sequences that are each at least 80% to 100% identical to at least one light chain variable region CDR1, CDR2, and CDR3 as set forth in any one of SEQ ID NOS:373-434 and 799-804, respectively, wherein each CDR has from zero to three amino acid changes, and comprises a heavy chain variable region containing CDR1, CDR2, and CDR3 sequences that are each at least 80% to 100% identical to at least one heavy chain variable region CDR1, CDR2, and CDR3 as set forth in any one of SEQ ID NOS:435-496 and 805-810, respectively, wherein each CDR has from zero to three amino acid changes.

### Polynucleotides, Expression Vectors, and Host Cells

This disclosure provides polynucleotides (isolated or purified or pure polynucleotides) encoding the fusion proteins of this disclosure, vectors (including cloning vectors and expression vectors) comprising such polynucleotides, and cells (e.g., host cells) transformed or transfected with a polynucleotide or vector according to this disclosure.

In certain embodiments, a polynucleotide (DNA or RNA) encoding a binding domain of this disclosure, or a fusion protein containing one or more such binding domains is contemplated. Expression cassettes encoding SMIP, PIMS or other fusion constructs are provided in the examples appended hereto.

The present disclosure also relates to vectors that include a polynucleotide of this disclosure and, in particular, to recombinant expression constructs. In one embodiment, this disclosure contemplates a vector comprising a polynucleotide encoding a binding domain of this disclosure or a polypeptide comprising such a binding domain, e.g., a SMIP, PIMS, SCORPION, Xceptor or other mono, bi- or multi-functional fusion protein, along with other polynucelotide sequences that cause or facilitate transcription, translation, and processing of such binding domain-encoding sequences.

Appropriate cloning and expression vectors for use with prokaryotic and eukaryotic hosts are described, for example, in Sambrook et al., Molecular Cloning: A Laboratory Manual, Second Edition, Cold Spring Harbor, N.Y., (1989). Exemplary cloning/expression vectors include cloning vectors, shuttle vectors, and expression constructs, that may be based on plasmids, phagemids, phasmids, cosmids, viruses, artificial chromosomes, or any nucleic acid vehicle known in the art suitable for amplification, transfer, and/or expression of a polynucleotide contained therein

As used herein, “vector” means a nucleic acid molecule capable of transporting another nucleic acid to which it has been linked. Exemplary vectors include plasmids, yeast artificial chromosomes, and viral genomes.

Certain vectors can autonomously replicate in a host cell, while other vectors can be integrated into the genome of a host cell and thereby are replicated with the host genome. In addition, certain vectors are referred to herein as “recombinant expression vectors” (or simply, “expression vectors”), which contain nucleic acid sequences that are operatively linked to an expression control sequence and, therefore, are capable of directing the expression of those sequences.

In certain embodiments, expression constructs are derived from plasmid vectors. Illustrative constructs include modified pNASS vector (Clontech, Palo Alto, Calif.), which has nucleic acid sequences encoding an ampicillin resistance gene, a polyadenylation signal and a T7 promoter site; pDEF38 and pNEF38 (CMC ICOS Biologics, Inc.), which have a CHEF1 promoter; and pD18 (Lonza), which has a CMV promoter. Other suitable mammalian expression vectors are well known (see, e.g., Ausubel et al., 1995; Sambrook et al., supra; see also, e.g., catalogs from Invitrogen, San Diego, Calif.; Novagen, Madison, Wis.; Pharmacia, Piscataway, N.J.). Useful constructs may be prepared that include a dihydrofolate reductase (DHFR)-encoding sequence under suitable regulatory control, for promoting enhanced production levels of the fusion proteins, which levels result from gene amplification following application of an appropriate selection agent (e.g., methotrexate).

Generally, recombinant expression vectors will include origins of replication and selectable markers permitting transformation of the host cell, and a promoter derived from a highly-expressed gene to direct transcription of a downstream structural sequence, as described above. A vector in operable linkage with a polynucleotide according to this disclosure yields a cloning or expression construct. Exemplary cloning/expression constructs contain at least one expression control element, e.g., a promoter, operably linked to a polynucleotide of this disclosure. Additional expression control elements, such as enhancers, factor-specific binding sites, terminators, and ribosome binding sites are also contemplated in the vectors and cloning/expression constructs according to this disclosure. The heterologous structural sequence of the polynucleotide according to this disclosure is assembled in appropriate phase with translation initiation and termination sequences. Thus, for example, the fusion protein-encoding nucleic acids as provided herein may be included in any one of a variety of expression vector constructs as a recombinant expression construct for expressing such a protein in a host cell.

The appropriate DNA sequence(s) may be inserted into a vector, for example, by a variety of procedures. In general, a DNA sequence is inserted into an appropriate restriction endonuclease cleavage site(s) by procedures known in the art. Standard techniques for cloning, DNA isolation, amplification and purification, for enzymatic reactions involving DNA ligase, DNA polymerase, restriction endonucleases and the like, and various separation techniques are contemplated. A number of standard techniques are described, for example, in Ausubel et al. (1993 Current Protocols in Molecular Biology, Greene Publ. Assoc. Inc. & John Wiley & Sons, Inc., Boston, Mass.); Sambrook et al. (1989 Molecular Cloning, Second Ed., Cold Spring Harbor Laboratory, Plainview, N.Y.); Maniatis et al. (1982 Molecular Cloning, Cold Spring Harbor Laboratory, Plainview, N.Y.); Glover (Ed.) (1985 DNA Cloning Vol. I and II, IRL Press, Oxford, UK); Hames and Higgins (Eds.), (1985 Nucleic Acid Hybridization, IRL Press, Oxford, UK); and elsewhere.

The DNA sequence in the expression vector is operatively linked to at least one appropriate expression control sequence (e.g., a constitutive promoter or a regulated promoter) to direct mRNA synthesis. Representative examples of such expression control sequences include promoters of eukaryotic cells or their viruses, as described above. Promoter regions can be selected from any desired gene using CAT (chloramphenicol transferase) vectors or other vectors with selectable markers. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art, and preparation of certain particularly preferred recombinant expression constructs comprising at least one promoter or regulated promoter operably linked to a nucleic acid encoding a protein or polypeptide according to this disclosure is described herein.

Variants of the polynucleotides of this disclosure are also contemplated. Variant polynucleotides are at least 90%, and preferably 95%, 99%, or 99.9% identical to one of the polynucleotides of defined sequence as described herein, or that hybridizes to one of those polynucleotides of defined sequence under stringent hybridization conditions of 0.015M sodium chloride, 0.0015M sodium citrate at about 65-68° C. or 0.015M sodium chloride, 0.0015M sodium citrate, and 50% formamide at about 42° C. The polynucleotide variants retain the capacity to encode a binding domain or fusion protein thereof having the functionality described herein.

The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing are 0.015M sodium chloride, 0.0015M sodium citrate at about 65-68° C. or 0.015M sodium chloride, 0.0015M sodium citrate, and 50% formamide at about 42° C. (see Sambrook et al., Molecular Cloning: A Laboratory Manual, 2nd Ed., Cold Spring Harbor Laboratory, Cold Spring Harbor, N.Y. 1989).

More stringent conditions (such as higher temperature, lower ionic strength, higher formamide, or other denaturing agent) may also be used; however, the rate of hybridization will be affected. In instances wherein hybridization of deoxyoligonucleotides is concerned, additional exemplary stringent hybridization conditions include washing in 6×SSC, 0.05% sodium pyrophosphate at 37° C. (for 14-base oligonucleotides), 48° C. (for 17-base oligonucleotides), 55° C. (for 20-base oligonucleotides), and 60° C. (for 23-base oligonucleotides).

A further aspect of this disclosure provides a host cell transformed or transfected with, or otherwise containing, any of the polynucleotides or vector/expression constructs of this disclosure. The polynucleotides or cloning/expression constructs of this disclosure are introduced into suitable cells using any method known in the art, including transformation, transfection and transduction. Host cells include the cells of a subject undergoing ex vivo cell therapy including, for example, ex vivo gene therapy. Eukaryotic host cells contemplated as an aspect of this disclosure when harboring a polynucleotide, vector, or protein according to this disclosure include, in addition to a subject's own cells (e.g., a human patient's own cells), VERO cells, HeLa cells, Chinese hamster ovary (CHO) cell lines (including modified CHO cells capable of modifying the glycosylation pattern of expressed multivalent binding molecules, see US Patent Application Publication No. 2003/0115614), COS cells (such as COS-7), W138, BHK, HepG2, 3T3, RIN, MDCK, A549, PC12, K562, HEK293 cells, HepG2 cells, N cells, 3T3 cells, Spodoptera frugiperda cells (e.g., Sf9 cells), Saccharomyces cerevisiae cells, and any other eukaryotic cell known in the art to be useful in expressing, and optionally isolating, a protein or peptide according to this disclosure. Also contemplated are prokaryotic cells, including Escherichia coli, Bacillus subtilis, Salmonella typhimurium, a Streptomycete, or any prokaryotic cell known in the art to be suitable for expressing, and optionally isolating, a protein or peptide according to this disclosure. In isolating protein or peptide from prokaryotic cells, in particular, it is contemplated that techniques known in the art for extracting protein from inclusion bodies may be used. The selection of an appropriate host is within the scope of those skilled in the art from the teachings herein. Host cells that glycosylate the fusion proteins of this disclosure are contemplated.

The term “recombinant host cell” (or simply “host cell”) refers to a cell containing a recombinant expression vector. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

A recombinant host cells can be cultured in a conventional nutrient medium modified as appropriate for activating promoters, selecting transformants, or amplifying particular genes. The culture conditions for particular host cells selected for expression, such as temperature, pH and the like, will be readily apparent to the ordinarily skilled artisan. Various mammalian cell culture systems can also be employed to express recombinant protein. Examples of mammalian expression systems include the COS-7 lines of monkey kidney fibroblasts, described by Gluzman (1981) Cell 23:175, and other cell lines capable of expressing a compatible vector, for example, the C127, 3T3, CHO, HeLa and BHK cell lines. Mammalian expression vectors will comprise an origin of replication, a suitable promoter and, optionally, enhancer, and also any necessary ribosome binding sites, polyadenylation site, splice donor and acceptor sites, transcriptional termination sequences, and 5′-flanking nontranscribed sequences, for example, as described herein regarding the preparation of multivalent binding protein expression constructs. DNA sequences derived from the SV40 splice, and polyadenylation sites may be used to provide the required nontranscribed genetic elements. Introduction of the construct into the host cell can be effected by a variety of methods with which those skilled in the art will be familiar, including calcium phosphate transfection, DEAE-Dextran-mediated transfection, or electroporation (Davis et al. (1986) Basic Methods in Molecular Biology).

In one embodiment, a host cell is transduced by a recombinant viral construct directing the expression of a protein or polypeptide according to this disclosure. The transduced host cell produces viral particles containing expressed protein or polypeptide derived from portions of a host cell membrane incorporated by the viral particles during viral budding.

### Compositions and Methods of Use

To treat human or non-human mammals suffering a disease state associated with TNF-α and IL6 dysregulation, a composition comprising a TNF-α antagonist binding protein and an IL6 antagonist binding protein of this disclosure is administered to the subject in an amount that is effective to ameliorate symptoms of the disease state following a course of one or more administrations. Being polypeptides, the binding proteins of this disclosure can be suspended or dissolved in a pharmaceutically acceptable diluent, optionally including a stabilizer of other pharmaceutically acceptable excipients, which can be used for intravenous administration by injection or infusion, as more fully discussed below.

A pharmaceutically effective dose is that dose required to prevent, inhibit the occurrence of, or treat (alleviate a symptom to some extent, preferably all symptoms of) a disease state. The pharmaceutically effective dose depends on the type of disease, the composition used, the route of administration, the type of subject being treated, the physical characteristics of the specific subject under consideration for treatment, concurrent medication, and other factors that those skilled in the medical arts will recognize. For example, an amount between 0.1 mg/kg and 100 mg/kg body weight (which can be administered as a single dose, or in multiple doses given hourly, daily, weekly, monthly, or any combination thereof that is an appropriate interval) of active ingredient may be administered depending on the potency of a binding domain polypeptide or multi-specific protein fusion of this disclosure.

In certain aspects, compositions of antagonist binding proteins are provided by this disclosure. Compositions of this disclosure generally comprise one or more type of binding domain protein in combination with a pharmaceutically acceptable carrier, excipient, or diluent. Such carriers will be nontoxic to recipients at the dosages and concentrations employed. Pharmaceutically acceptable carriers for therapeutic use are well known in the pharmaceutical art, and are described, for example, in Remington's Pharmaceutical Sciences, Mack Publishing Co. (A. R. Gennaro (Ed.) 1985). For example, sterile saline and phosphate buffered saline at physiological pH may be used. Preservatives, stabilizers, dyes and the like may be provided in the pharmaceutical composition. For example, sodium benzoate, sorbic acid, or esters of p-hydroxybenzoic acid may be added as preservatives. Id. at 1449. In addition, antioxidants and suspending agents may be used. Id. The compounds of the present disclosure may be used in either the free base or salt forms, with both forms being considered as being within the scope of the present disclosure.

Pharmaceutical compositions may also contain diluents such as buffers, antioxidants such as ascorbic acid, low molecular weight (less than about 10 residues) polypeptides, proteins, amino acids, carbohydrates (e.g., glucose, sucrose, or dextrins), chelating agents (e.g., EDTA), glutathione or other stabilizers or excipients. Neutral buffered saline or saline mixed with nonspecific serum albumin are exemplary appropriate diluents. Preferably, product is formulated as a lyophilizate using appropriate excipient solutions as diluents.

In certain embodiments, cis-signaling of IL6 is minimally or not inhibited, i.e., any inhibition of cis-signaling is not substantial, meaning that inhibition is non-existent, asymptomatic, or not detectable. The extent of inhibition of IL6 trans-signaling can vary, but in general trans-signaling is altered to an extent that has a positive effect on symptoms of a disease state mediated by or associated with such signaling. In certain embodiments, inhibition of trans-signaling of IL6 by binding domain polypeptides or fusion proteins thereof of this disclosure can retard, stop, or reverse disease progression.

Compositions of this disclosure can be used to treat disease states in human and non-human mammals that are mediated by TNF-α and IL6 signaling.

Increased production of IL-6, and thus IL-6 signaling, has been implicated in various disease processes, including Alzheimer's disease, autoimmunity (e.g., rheumatoid arthritis, SLE), inflammation, myocardial infarction, Paget's disease, osteoporosis, solid tumors (e.g., colon cancer, RCC prostatic and bladder cancers), certain neurological cancers, B-cell malignancies (e.g., Castleman's disease, some lymphoma subtypes, chronic lymphocytic leukemia, and, in particular, malignant melanoma). In some instances, IL-6 is implicated in proliferation pathways because it acts with other factors, such as heparin-binding epithelial growth factor and hepatocyte growth factor (see, e.g., Grant et al. (2002) Oncogene 21:460; Badache and Hynes (2001) Cancer Res. 61:383; Wang et al. (2002) Oncogene 21:2584). Similarly, the TNF superfamily is known to be involved in a variety of disorders, such as cancer (tumorigenesis, including proliferation, migration, metastasis), autoimmunity (SLE, diabetes), chronic heart failure, bone resorption, and atherosclerosis, to name a few (see, e.g., Aggarwal (2003) Nature Rev. 3:745; Lin et al. (2008) Clin. Immunol. 126:13).

Compositions comprising the antagonist binding domains of this disclosure are useful in treating autoimmune and other disorders including, rheumatoid arthritis, psoriasis, systemic lupus erythematosus (SLE), and inflammatory bowel disease (IBD), including Crohn's disease and ulcerative colitis.

Compositions comprising the TNF-α antagonist binding protein and the IL6 antagonist binding protein of this disclosure may be administered simultaneously (in the same formulation or concurrently in separate formulations, with concurrently referring to agents administered within 30 minutes of each other) or sequentially (from 30 minutes up to one week following administration of the first antagonist binding protein).

Also contemplated is the administration of binding protein compositions of this disclosure in combination with a second agent. A second agent may be one accepted in the art as a standard treatment for a particular disease state, such as inflammation and autoimmunity. Exemplary second agents contemplated include cytokines, growth factors, steroids, NSAIDs, DMARDs, chemotherapeutics, radiotherapeutics, or other active and ancillary agents, or any combination thereof.

“Pharmaceutically acceptable salt” refers to a salt of a binding domain polypeptide or fusion protein of this disclosure that is pharmaceutically acceptable and that possesses the desired pharmacological activity of the parent compound. Such salts include the following: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine, or the like.

In particular illustrative embodiments, a binding protein of this disclosure is administered intravenously by, for example, bolus injection or infusion. Routes of administration in addition to intravenous include oral, topical, parenteral (e.g., sublingually or buccally), sublingual, rectal, vaginal, intranasal, and perispinal. The term parenteral as used herein includes subcutaneous injections, intravenous, intramuscular, intrasternal, intracavernous, intrathecal, intrameatal, intraurethral injection or infusion techniques. The pharmaceutical composition is formulated so as to allow the active ingredients contained therein to be bioavailable upon administration of the composition to a patient. Compositions that will be administered to a patient take the form of one or more dosage units, where for example, a tablet may be a single dosage unit, and a container of one or more compounds of this disclosure in aerosol form may hold a plurality of dosage units.

For oral administration, an excipient and/or binder may be present, such as sucrose, kaolin, glycerin, starch dextrans, cyclodextrins, sodium alginate, ethyl cellulose, and carboxy methylcellulose. Sweetening agents, preservatives, dye/colorant, flavor enhancer, or any combination thereof may optionally be present. A coating shell may also optionally be used.

In a composition intended to be administered by injection, one or more of a surfactant, preservative, wetting agent, dispersing agent, suspending agent, buffer, stabilizer, isotonic agent, or any combination thereof may optionally be included.

For nucleic acid-based formulations, or for formulations comprising expression products according to this disclosure, about 0.01 μg/kg to about 100 mg/kg body weight will be administered, for example, by the intradermal, subcutaneous, intramuscular, or intravenous route, or by any route known in the art to be suitable under a given set of circumstances. A preferred dosage, for example, is about 1 μg/kg to about 20 mg/kg, with about 5 μg/kg to about 10 mg/kg particularly preferred. It will be evident to those skilled in the art that the number and frequency of administration will be dependent upon the response of the host.

The pharmaceutical compositions of this disclosure may be in any form that allows for administration to a patient, such as, for example, in the form of a solid, liquid, or gas (aerosol). The composition may be in the form of a liquid, e.g., an elixir, syrup, solution, emulsion or suspension, for administration by any route described herein.

A liquid pharmaceutical composition as used herein, whether in the form of a solution, suspension or other like form, may include one or more of the following components: sterile diluents such as water for injection, saline solution (e.g., physiological saline), Ringer's solution, isotonic sodium chloride, fixed oils such as synthetic mono- or digylcerides that may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; buffers such as acetates, citrates or phosphates; chelating agents such as ethylenediaminetetraacetic acid; and agents for the adjustment of tonicity such as sodium, chloride, or dextrose. The parenteral preparation can be enclosed in ampoules, disposable syringes or multiple dose vials made of glass or plastic. Physiological saline is a preferred additive. An injectable pharmaceutical composition is preferably sterile.

It may also be desirable to include other components in the preparation, such as delivery vehicles including aluminum salts, water-in-oil emulsions, biodegradable oil vehicles, oil-in-water emulsions, biodegradable microcapsules, and liposomes. Examples of adjuvants for use in such vehicles include N-acetylmuramyl-L-alanine-D-isoglutamine (MDP), lipopolysaccharides (LPS), glucan, IL-12, GM-CSF, γ-interferon, and IL-15.

While any suitable carrier known to those of ordinary skill in the art may be employed in the pharmaceutical compositions of this disclosure, the type of carrier will vary depending on the mode of administration and whether a sustained release is desired. For parenteral administration, the carrier may comprise water, saline, alcohol, a fat, a wax, a buffer, or any combination thereof. For oral administration, any of the above carriers or a solid carrier, such as mannitol, lactose, starch, magnesium stearate, sodium saccharine, talcum, cellulose, glucose, sucrose, magnesium carbonate, or any combination thereof, may be employed.

This disclosure contemplates a dosage unit comprising a pharmaceutical composition of this disclosure. Such dosage units include, for example, a single-dose or a multi-dose vial or syringe, including a two-compartment vial or syringe, one comprising the pharmaceutical composition of this disclosure in lyophilized form and the other a diluent for reconstitution. A multi-dose dosage unit can also be, e.g., a bag or tube for connection to an intravenous infusion device.

This disclosure also contemplates a kit comprising a pharmaceutical composition in a unit dose or multi-dose container, e.g., a vial, and a set of instructions for administering the composition to patients suffering a disorder as described herein.

All U.S. patents, U.S. patent application publications, U.S. patent applications, foreign patents, foreign patent applications, non-patent publications, tables, sequences, webpages, or the like referred to in this specification, are incorporated herein by reference, in their entirety. The following examples are intended to illustrate, but not limit, this disclosure.

### EXAMPLES

### Sequences:

Amino acid sequences of exemplary SMIP molecules having an anti-IL6xR binding domain are provided in SEQ ID NOS:671-694, with the corresponding nucleotide expression cassettes of the fusion proteins being provided in SEQ ID NOS:761-784 (note the mature proteins will lack the signal peptide sequence found in SEQ ID NOS:671-694). Specifically, SMIP fusion proteins TRU(S6)-1004, 1007, 1008, 1013, 1018, 1019, 1029, and 1038 are provided in SEQ ID NOs:672, 673, 674, 676, 678, 679, 684 and 685, respectively.

Amino acid sequences of exemplary TNF-α antagonists comprise amino acids 23-257 of SEQ ID NO:846 or amino acids 31-211 of SEQ ID NO:847 (with or without the native leader peptide sequence included in these sequence).

### Example 1

### Pretreatment of Mice with TNF-α Antagonist and Anti-mIL-6 Blocks Early Biomarker Response to Exogenous mTNF-α More Potently than Pretreatment of Mice with Either Alone

In this Example, A2-XT6 (a multi-target binding molecule with the TNFR extracellular domain (ECD) at the N-terminal end and the anti-hyperlL6 scFv at the carboxy-terminal end; SEQ ID NO:608) plus anti-mIL-6 antibody—mediated blockade of biomarker response in normal BALB/c mice was investigated. Note that A2-XT6 binds to human IL6 but does not bind to murine IL6. Accordingly, in this example, this molecule functions as a TNF-α antagonist only.

The ability of TNF-α and IL-6 antagonist proteins disclosed herein to reduce TNF-a-induced production of serum amyloid A (SAA) protein in mice was examined as described below. Serum amyloid A (SAA) protein is one of the major acute-phase proteins in humans and mice. Prolonged elevation of plasma SAA levels is found in chronic inflammation and leads to amyloidosis which affects the liver, kidney and spleen (Rienhoff et al. (1990) Mol. Biol. Med. 7:287). Both IL-6 and TNF have been shown to induce SAA when administered alone (Benigni et al., (1996) Blood 87:1851; Ramadori et al., (1988) Eur. J. Immunol. 18:1259).

Experiments were designed as outlined below in Table 1 to determine whether pretreatment of mice with A2-XT6 and anti-mIL-6 antibody given in combination can block the biomarker response to exogenous mTNF-α more potently than pretreatment of mice with A2-XT6 only.

TABLE 1
Study Design

| | | | | | Timepoint | |
|---|---|---|---|---|---|---|
| | Retro-orbital | IV | IP | IP | (hr after | |
| | Injection | Dose | Injection | Dose | mTNF-a | # |
| Group | At T = −1 hr | (ug) | At T = 0 hr | (ug) | injection) | mice |
| G-1 | PBS | NA | PBS | NA | 2 hr & 24 | 4 + 4 |
| | | | | | hr | |
| G-2 | PBS | NA | mTNF-a | 0.5 | 2 hr & 24 | 4 + 4 |
| | | | | | hr | |
| G-3 | A2-XT6 | 300 | mTNF-a | 0.5 | 2 hr & 24 | 4 + 4 |
| | | | | | hr | |
| G-4 | A2-XT6 + | 300 + | mTNF-a | 0.5 | 2 hr & 24 | 4 + 4 |
| | anti-mIL-6 Ab | 200 | | | hr | |
| G-5 | ENBREL ® | 200 | mTNF-a | 0.5 | 2 hr & 24 | 4 + 4 |
| | | | | | hr | |
| G-6 | ENBREL ® + | 200 + | mTNF-a | 0.5 | 2 hr & 24 | 4 + 4 |
| | anti-mIL-6 Ab | 200 | | | hr | |
| G-7 | Anti-mIL-6 Ab | 200 | mTNF-a | 0.5 | 2 hr & 24 | 4 + 4 |
| | | | | | hr | |
| | | | | | | 56 |

Mice were female BALB/C; 12.5 weeks of age

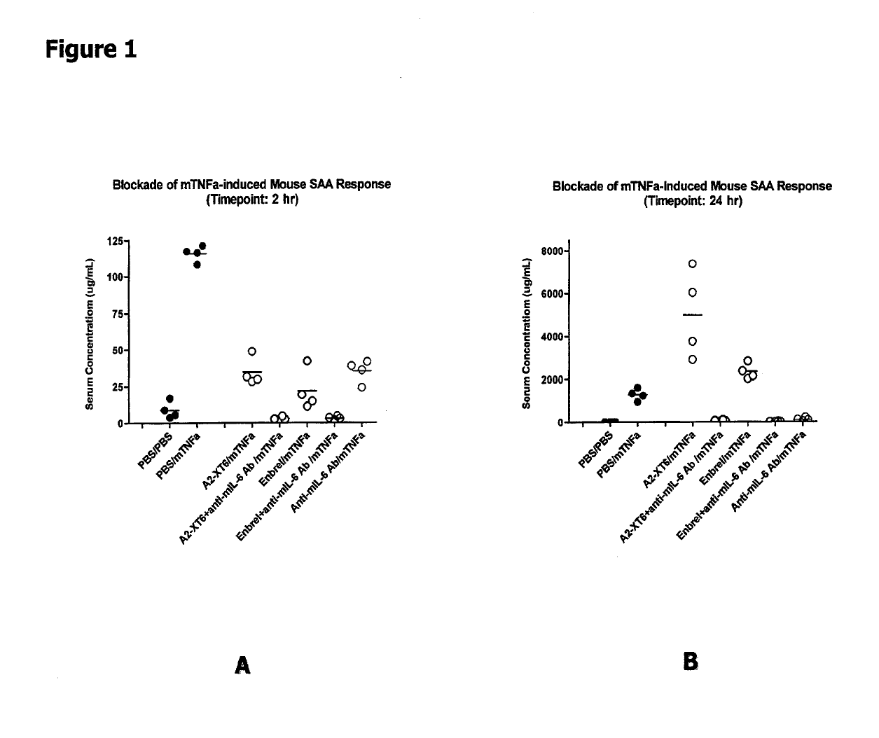

Readouts were mouse serum amyloid A (SAA) ELISA (see FIGS. 1A and 1B). Other readouts may also be carried out, such as: Mouse Cytokine/Chemokine Luminex Assay (2 hr timepoint only); (TNF-α, Eotaxin, G-CSF, IL-1a, IL-6, IP-10, KC. MCP-1, MIP-1B, MIG, and RANTES); Mouse Soluble Cytokine Receptor Luminex Assay; (sIL-1R1, sIL-6R, sgp130, sTNFR1, and sTNFR2).

The following reagents were used for these experiments: ENBREL®: Amgen/Wyeth, Lot #P109374, 25mg/0.5mL; A2-XT6: Trubion, Zeu080a, Lot #867MBD090505, and endotoxin level: <0.051 EU/mg; Anti-mIL-6 antibody (clone:MP520F3): R&D, cat#MAB406, and endotoxin level: 0.33 EU/mg; Mouse TNF-α: R&D, Cat #410-MT/CF, Lot #CS080807A, and endotoxin level: 3.12 EU/mg; PBS: Gibco, Cat #14190-136.

As shown in FIG. 1, panel A, pretreatment of mice with A2-XT6 and anti-mIL-6 antibody given in combination results in lower SAA than in mice pretreated with either antagonist alone, indicating that such pretreatment can block the biomarker response to exogenous mTNF-α more potently than pretreatment of mice with A2-XT6 or anti-mIL-6 antibody alone at 2 hours post-TNF-alpha injection. The block observed was more effective than ENBREL® alone and as effective as pretreatment with ENBREL® in combination with anti-mIL-6 antibody at 2 hours post-injection. Lower SAA levels were still observed at 24 hours post-TNF-alpha injection in mice pretreated with A2-XT6 and anti-mIL-6 antibody as compared to A2-XT6 alone. However, serum SAA levels at 24 hours post-injection for mice pretreated with anti-mIL-6 antibody alone were equivalent to those observed in mice pretreated with the combination of antagonists.

These results indicate that pretreatment with the combination of a TNF-α antagonist and an IL-6 antagonist provides an effective early block of the signaling response to exogenous TNF-α that is more potent than pretreatment with either antagonist alone. Accordingly, these results provide support for the use of the combination therapy in a variety of autoimmune settings associated with TNF-α and IL6, including rheumatoid arthritis, SLE, psoriasis, inflammatory bowel disease and other such disorders.

### Example 2

### Animal Models for Rheumatoid Arthritis

The therapeutic efficacy of any of the compositions comprising antagonist binding molecules disclosed herein is examined in at least one of two murine models of rheumatoid arthritis (RA), namely the collagen induced arthritis (CIA) and glucose-6-phosphate isomerase (G6PI) models. Each of these models has been shown to be useful for predicting efficacy of certain classes of therapeutic drugs in RA (see Holmdahl (2000) Arthritis Res. 2:169; Holmdahl (2006) Immunol. Lett. 103:86; Holmdahl (2007) Methods Mol. Med. 136:185; McDevitt, H. (2000) Arthritis Res. 2:85; Kamradt and Schubert (2005) Arthritis Res. Ther. 7:20).

### (i) CIA Model

The CIA model is a well-characterized mouse model of arthritis in terms of its pathogenesis and immunological basis. In addition, it is a widely used model of RA and is an acceptable model to persons of skill in the art for investigating potential new therapeutics for RA (Jirholt et al. (2001) Arthritis Res. 3:87; Van den Berg, W. B. (2002) Curr. Rheumatol. Rep. 4:232; Rosloniec (2003) Collagen-Induced Arthritis. In Current Protocols in Immunology, eds. Coligan et al., John Wiley & Sons, Inc, Hoboken, N.J.).

In the CIA model, arthritis is induced by immunization of male DBA/1 mice with collagen II (CII) in Complete Freund's Adjuvant (CFA). Specifically, mice are injected intradermally/subcutaneously with CII in CFA on Day -21 and boosted with CII in Incomplete Freund's Adjuvant (IFA) on Day 0. Mice develop clinical signs of arthritis within days of the boost with CII/IFA. A subset of mice (0% to 10%) immunized with CII/CFA develop signs of arthritis on or around Day 0 without a boost and are excluded from the experiments. In some CIA experiments, the boost is omitted and mice are instead treated with Xceptor or control starting 21 days after immunization with CII/CFA (i.e. the day of first treatment is Day 0).

Mice are treated with the compositions described herein, vehicle (PBS), or negative or positive control in a preventative and/or therapeutic regimen. Preventative treatment starts on Day 0 and continues through the peak of disease in control (untreated) mice. Therapeutic treatment starts when the majority of mice show mild signs of arthritis. ENBREL®, which has been shown to have good efficacy in both the CIA and G6PI-induced models of arthritis, is used as a positive control. Data collected in every experiment includes clinical scores and cumulative incidence of arthritis. Clinical signs of arthritis in the CIA model are scored using a scale from 0 to 4 as shown in Table 2 below.

TABLE 2

| Score | Observations |
|---|---|
| 0 | No apparent swelling or redness |
| 1 | Swelling/redness in one to three digits |
| 2 | Redness and/or swelling in more than three digits, mild |
| | swelling extending into the paw, swollen or red ankle, |
| | or mild swelling/redness of forepaw |
| 3 | Swollen paw with mild to moderate redness |
| 4 | Extreme redness and swelling in entire paw |

### (ii) G6PI Model

In the G6PI model, arthritis is induced by immunization of DBA/1 mice with G6PI in adjuvant (Kamradt and Schubert (2005) Arthritis Res. Ther. 7:20; Schubert et al., (2004) J. Immunol. 172:4503; Bockermann, R. et al. (2005) Arthritis Res. Ther. 7:R1316; Iwanami et al., (2008) Arthritis Rheum. 58:754; Matsumoto et al., (2008) Arthritis Res. Ther. 10:R66). G6PI is an enzyme present in virtually all cells in the body and it is not known why immunization induces a joint specific disease. A number of agents, such as CTLA4-Ig, TNF antagonists (e.g. ENBREL®) and anti-IL6 receptor monoclonal antibody, have been shown to inhibit development of arthritis in the G6PI model.

Male DBA/1 mice are immunized with G6PI in Complete Freund's Adjuvant (CFA) in order to induce arthritis. Specifically, mice are injected intradermally/subcutaneously with G6PI in CFA on Day 0 and develop clinical signs of arthritis within days of the immunization. As with the CIA model discussed above, mice are treated with antagonists as described herein, vehicle (PBS), or negative or positive control in a preventative and/or therapeutic regimen. Preventative treatment starts on Day 0 and continues through the peak of disease in control mice. Therapeutic treatment starts when the majority of mice show mild signs of arthritis. ENBREL®, which has been shown to have good efficacy in both the CIA and G6PI-induced models of arthritis, is used as a positive control. Data collected in every experiment includes clinical scores and cumulative incidence of arthritis. Clinical signs of arthritis in the G6PI model are scored using a scale similar to that employed for the CIA model.

### Example 3

### In Vivo Efficacy of TNF-α+IL-6 Antagonists in a Murine Model for Rheumatoid Arthritis

The G6PI model as described in Example 2 was used to study the in vivo efficacy of the combination of a TNF-α antagonist (ENBREL®) and an IL-6 antagonist (anti-mIL6 antibody) in reducing severity of arthritis symptoms.

The randomized and blinded study was designed as follows: on Day −3, ID and temperature transponders were implanted. On Day −1 mice were weighed, temperatures taken, and mice were randomized. On Day 0 mice were weighed, temperatures taken; indicated emulsion was injected (G6PI and CFA) subcutaneously in the base of the tail. After immunization, mice were injected with the indicated amount of protein (RITUXAN®, ENBREL®, anti-mouse IL-6 (20F3), or combination of ENBREL® and anti-mouse IL-6). See Table 3 below.

TABLE 3
IP Dosing

| | | | | Number |
|---|---|---|---|---|
| Group | Treatment | IP Dosing (μg) | Days | of mice |
| A | Untreated | — | — | 5 |
| B | RITUXAN ® | 154 QOD | 0, 3, 5, 7, 10, 12, 14 | 12 |
| C | ENBREL ® | 100 QOD | 0, 3, 5, 7, 10, 12, 14 | 12 |
| D | | 10 QOD | 0, 3, 5, 7, 10, 12, 14 | 12 |
| E | Anti-mouse IL-6 | 154 QOD | 0, 3, 5, 7, 10, 12, 14 | 12 |
| | (20F3) | | | |
| F | | 15.4 QOD | 0, 3, 5, 7, 10, 12, 14 | 12 |
| G | ENBREL ® + | 10 QOD | 0, 3, 5, 7, 10, 12, 14 | 12 |
| | anti-mouse IL-6 | (Enbrel ®) | | |
| | | 15.4 QOD | | |
| | | (anti-mouse | | |
| | | IL-6) | | |
| H | Unprimed (naïve) | — | — | 5 |

Weights and temperatures were recorded 6 days per week. Arthritis was scored 3 times per week (or more if necessary). Arthritis scores were determined by grading each paw and adding the four scores together. Scores ranged from 0 (no arthritis) to 4 (severe swelling and redness). A mouse must have had a clinical score of 2 or more on two consecutive scoring sessions in order to have arthritis. A mouse was considered to have moderate to severe arthritis if it had a total paw score of 10 or more at any point during the observation period.

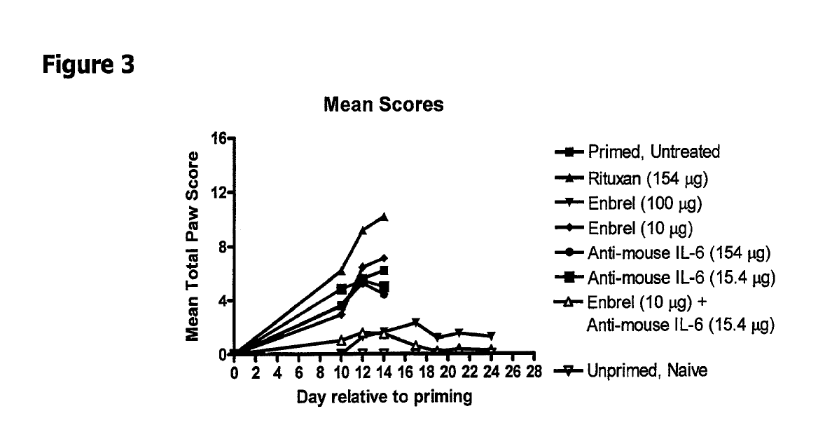

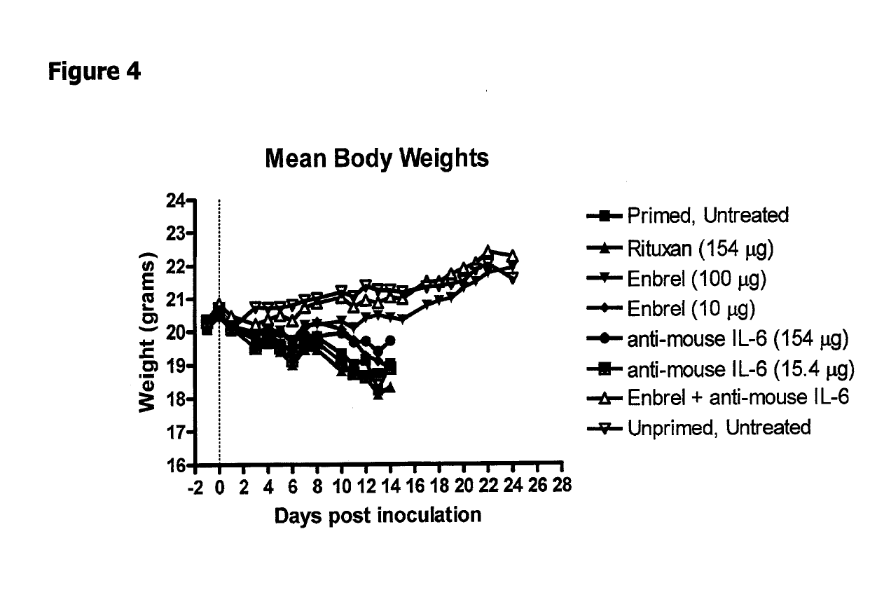

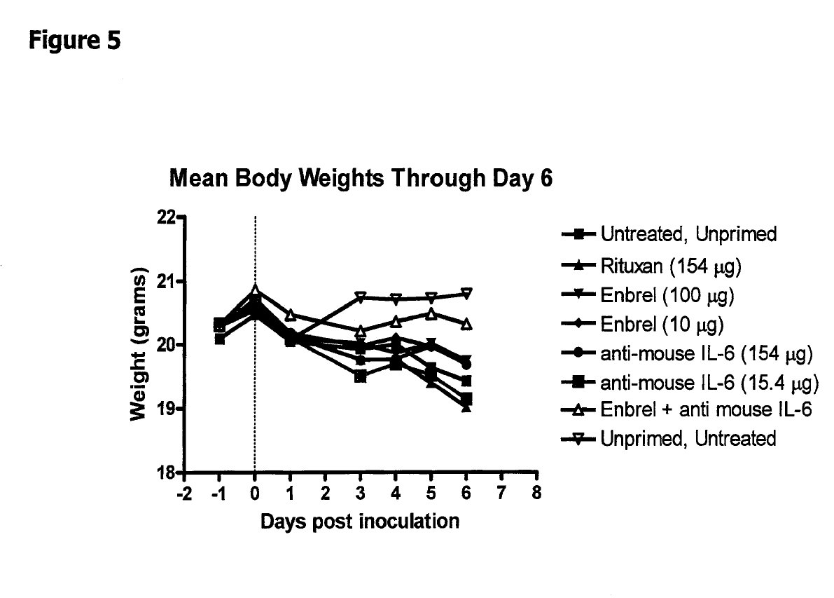

As shown in FIG. 3, the mean level of arthritis observed in mice treated with the combination of ENBREL® and anti-mIL6 antibody was reduced as compared to control mice and was slightly lower than that observed in mice treated with 100 μg ENBREL® alone. Similarly, as shown in FIGS. 4 and 5, mean body weight in mice treated with the combination of ENBREL® and anti-mIL6 antibody was maintained. The combination was as effective for preventing weight loss as 100 μg ENBREL®. At earlier time points in the study (e.g., Day 6), the combination treatment was more effective than 100 μg ENBREL® at preventing weight loss.

The study results are summarized in Table 4 below. In particular, treatment with a combination of ENBREL® at 10 μg and anti-mIL6 antibody significantly reduced the incidence of arthritis as compared to treatment with 100 μg ENBREL® alone.

TABLE 4
G6PI Model Study Results—Summary

| | | | Incidence of | | Number of |
|---|---|---|---|---|---|
| | Incidence | p Value vs | Moderate to | p Value vs | Mice |
| | of Arthritis | Control | Severe | Control | Euthanized |
| Group | (%) | Group | Arthritis % | Group | (Reason) |
| Untreated | 4/5 | N/A | 1/5 | N/A | 1 |
| | (92%) | | (20%) | | Severe |
| | | | | | arthritis |
| RITUXAN ® | 11/12 | N/A | 6/12 | N/A | 4 |
| | (92%) | | (50%) | | Severe |
| | | | | | arthritis |
| ENBREL ® | 3/12 | 0.0028** | 1/12 | 0.0686 | 0 |
| (100 μg) | (25%) | | (8%) | | |
| ENBREL ® | 11/12 | 1.0000 | 3/12 | 0.4003 | 2 |
| (10 μg) | (92%) | | (25%) | | Severe |
| | | | | | arthritis |
| Anti-mouse IL-6 | 8/12 | 0.3168 | 3/12 | 0.4003 | 1 |
| (154 μg) | (67%) | | (25%) | | Severe |
| | | | | | arthritis |
| Anti-mouse IL-6 | 11/12 | 1.0000 | 2/12 | 0.1930 | 1 |
| (15.4 μg) | (92%) | | (17%) | | Severe |
| | | | | | arthritis |
| ENBREL ® | 4/12 | 0. 0094** | 0/12 | 0.0137** | 0 |
| (10 μg) + anti- | (33%) | | (0%) | | |
| mouse IL-6 | | | | | |
| (15.4 μg) | | | | | |

p Value is from a contingency analysis (Fisher's exact test) for indicated group
versus the RITUXAN ® group.

Thus, these studies show that treatment with a combination of TNF-α antagonist and IL6 antagonist is much more effective at reducing severity and incidence of arthritis when compared to either therapeutic administered alone in a well-established pre-clinical animal model. Such combination treatment may be effective for the treatment of a variety of autoimmune diseases associated with TNF-α and IL6 in humans.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method for treating an autoimmune disease comprising administering to a subject in need thereof a therapeutically effective amount of a combination of an IL-6 antagonist and a TNF-α antagonist.

2. The method of claim 1 wherein the autoimmune disease is selected from the group consisting of rheumatoid arthritis, systemic lupus erythematosus (SLE), psoriasis, and inflammatory bowel disease.

3. (canceled)

4. (canceled)

5. The method of claim 1 wherein the IL-6 antagonist comprises a binding domain specific for an IL6/IL6R (IL6xR) complex, said binding domain specific for IL6xR comprising an amino acid sequence at least 80% identical to one or more light chain variable regions as listed in SEQ ID NOs:373-434 and 799-804 and an amino acid sequence that is at least 80% identical to one or more of the heavy chain variable regions as listed in SEQ ID NOs:435-496 and 805-810.

6. The method of claim 1 wherein the TNF-α antagonist comprises a binding domain specific for TNF-α, wherein the binding domain specific for TNF-α comprises amino acids 23-257 of SEQ ID NO:846 or amino acids 31-211 of SEQ ID NO:847.

7. The method of claim 1 wherein the IL-6 antagonist comprises a binding domain specific for an IL6/IL6R (IL6xR) complex, said binding domain specific for IL6xR comprising an amino acid sequence at least 80% identical to one or more light chain variable regions as listed in SEQ ID NOs:373-434 and 799-804 and an amino acid sequence that is at least 80% identical to one or more of the heavy chain variable regions as listed in SEQ II) NOs:435-496 and 805-810; and
wherein the TNF-α antagonist comprises a binding domain specific for TNF-α and comprising amino acids 23-257 of SEQ. ID NO:846 or amino acids 31-211 of SEQ ID NO:847.

8. The method of claim 1 wherein the IL-6 antagonist and the TNF-α antagonist both comprise an antibody, or an antigen binding domain thereof, a Fab, or a scFv.

9. The method of claim 1 wherein the IL-6 antagonist is an antibody and the TNF-α antagonist is a small modular immunopharmaceutical (SMIP) protein.

10. The method of claim 1 wherein the IL-6 antagonist is a SMIP protein and the TNF-α antagonist is an antibody.

11. The method of claim 1 wherein the IL-6 antagonist and the TNF-α antagonist are both SMIP proteins.

12. The method of claim 7 wherein the binding domain of the IL-6 antagonist and the TNF-α antagonist are an antibody or antigen binding domain thereof, a Fab, or a scFv.

13. The method of claim 5 wherein the binding domain of the IL-6 antagonist comprises a light chain variable region containing CDR1, CDR2, and CDR3 sequences that are each at least 80% identical to at least one light chain variable region CDR1, CDR2, and CDR3 as set forth in SEQ ID NOS:373-434 and 799-804, respectively.

14. The method of claim 5 wherein the binding domain of the IL-6 antagonist comprises a heavy chain variable region containing CDR1, CDR2, and CDR3 sequences that are each at least 80% identical to at least one heavy chain variable region CDR1, CDR2, and CDR3 as set forth in SEQ ID NOS:435-496 and 805-810, respectively.

15. The method of claim 5 wherein the binding domain of the IL-6 antagonist comprises a light chain variable region containing CDR1, CDR2, and CDR3 sequences that are each at least 80% identical to at least one light chain variable region CDR1, CDR2, and CDR3 as set forth in SEQ ID NOS:373-434 and 799-804, respectively, and comprises a heavy chain variable region containing CDR1, CDR2, and CDR3 sequences that are each at least 80% identical to at least one heavy chain variable region CDR1, CDR2, and CDR3 as set forth in SEQ ID NOS:435-496 and 805-810, respectively.

16. The method of claim 5 wherein the IL6xR complex has an amino acid sequence as set forth in SEQ ID NO:606.

17. The method of claim 7 wherein the binding domains of the IL-6 antagonist and the TNF-α antagonist are fused to (a) an immunoglobulin Fc domain or one or more CH domains of an immunoglobulin Fc domain, or (b) a serum protein binding protein.

18. The method of claim 17 wherein the one or more CH domains of an immunoglobulin Fc domain comprises a CH2 constant region and CH3 constant region, preferably IgG1 CH2 and CH3 domains.

19. The method of claim 18 wherein,
I) the IL-6 antagonist comprises, from amino-terminus to carboxy-terminus, (a) the binding domain specific for IL6xR fused to a linker, (h) the linker fused to an immunoglobulin heavy chain CH2 constant region polypeptide, and (c) the CH2 constant region polypeptide fused to an immunoglobulin heavy chain CH3 constant region polypeptide; and
II) the TNF-α antagonist comprises, from amino-terminus to carboxy-terminus, (a) the binding domain specific for TNF-α fused to a linker, (b) the linker fused to an immunoglobulin heavy chain CH2 constant region polypeptide, and (c) the CH2 constant region polypeptide fused to an immunoglobulin heavy chain CH3 constant region polypeptide.

20. The method of claim 18 wherein,
I) the IL-6 antagonist comprises, from carboxyl-terminus to amino-terminus, (a) the polypeptide binding domain specific for IL6xR fused to a first linker, (b) the first linker fused to an immunoglobulin heavy chain CH3 constant region polypeptide, (c) the CH3 constant region polypeptide fused to an immunoglobulin heavy chain CH2 constant region polypeptide, and (d) the CH2 constant region polypeptide fused to a second linker; and
II) the TNF-α antagonist comprises, from carboxy-terminus to amino-terminus, (a) the binding domain specific for TNF-α fused to a first linker, (b) the first linker fused to an immunoglobulin heavy chain CH3 constant region polypeptide, (c) the CH3 constant region polypeptide fused to an immunoglobulin heavy chain CH2 constant region polypeptide, and (d) the CH2 constant region polypeptide fused to a second linker.

21. The method of claim 19 wherein the linker is an immunoglobulin hinge region polypeptide.

22. The method of claim 19 wherein the linker is selected from the group consisting of SEQ ID NO:497-604, 823-828, 830-845, and 851-1106.

23. The method of claim 7, wherein: i) the IL-6 antagonist is administered simultaneously with the TNF-α antagonist in the same formulation; ii) the IL-6 antagonist is administered concurrently with the TNF-α antagonist in separate formulations within 30 minutes of each other; iii) the IL-6 antagonist is administered one week up to 30 minutes prior to administration of the TNF-α antagonist; or iv) the IL-6 antagonist is administered 30 minutes up to one week subsequent to administration of TNF-α antagonist.

24. The method of claim 20, wherein the first linker is a type II C-lectin stalk region or an immunoglobulin hinge region polypeptide and the second linker is an immunoglobulin hinge region polypeptide.
